SEQUENCE LISTING (23 sequences)

SEQ ID NO: 1 - DNA, 2973 bp, Hordeum vulgare
  source 1..2973: mol_type = other DNA
tcctcgaggtgcgtttaccccacacagagtacactccaactccagtccaatccagcccactgccgcttctgcccgcccatcgtaccgtcgcccgccccgatcccggccgccgccatgtcgtcggcggtcgcgtcccccgcgtccttcctcgcgctcgcgtccgcctcgcccgggagatcatcacggaggagggcgagggtgggcgcgtcgccaacccgcgctggggccggcaggctgcaatggcggccgtcgccgctgcagcgcacggctcgcgacggagcggtggccgcgcgcgccgccgggatcgacgacgccgcgcccggtaggcagccccgcgctcgccgctatggcgccgccaccaaggtcgcggatcccgtcaagacgctcgatcgcgacgccgcggaaggtggtgggccgtccccgccggcaccgaggcaggacgccgcccgtctgccgagtaagaacggcacgctgatcaacggtgagaacaaacctaccggcggcggtggcgcgactaaagacagcgggctgcccacacccgcacgcgcgccccatctgtcaatccagaacagagtaccggtgaacggtgaaaacaaacataaggtcgcctcgccgccgaccagcatagtggatgtcgcgtctccgggttccgcagctaacatttccatcagtaacaaggtgccgccgtccgttgtcccagccaagaagacgccgccgtcgtccgttttcccggccaagaagacgctgccgtcgtccggctcaaattttgtgtcctcggcctctgctcccaggctggacactgtcagcgatgtggaacttgcacagaagaaggatgcgctgattgtcaaagaagctccaaaaccaaaggctctttcggcccctgcagcccccgctgtacaagaagacctttgggatttcaagaaatacattggtttcgaggagcccgtggaggccaaggatgatggctcggctgttgcagatgatgcgggttcctttgaacatcaccagaatcatgattccggacctttggcaggggagaacgtcatgaacgtggtcgtcgttgctgctgaatgttctccctggtgcaaaacaggtggtcttggagatgttgcgggtgctttgcccaaggctttggctaagagaggacatcgtgttatggttgtggtaccaaggtatggggactatgaggaagcctacgatgtcggagtccgaaaatactacaaggctgctggacaggatatggaagtgaattatttccatgcttatatcgatggagtggattttgtgttcattgacgctcctctcttccgacaccgtcagcaagacatttatgggggcagcagacaggaaattatgaagcgcatgattttgttctgcaaggccgctgtcgaggttccttggcacgttccatgcggcggtgtcccttacggggatggaaatctggtcttcattgcaaatgattggcacacggcactcctgcctgtctatctgaaagcatattacagggaccatggtttgatgcaatacagtcgctccgttatggtgatacataacatcgctcaccagggccgtggccctgtagatgaattcccgttcaccgagttgcctgagcactacctggaacacttcagactgtacgaccccgtcggcggtgagcacgccaactacttcgccgccggcctgaagatggcggaccaggttgtcgtcgtgagccccgggtacctgtgggagctgaagacggtggagggcggctgggggcttcacgacatcatacggcagaacgactggaagacccgcggcatcgtgaacggcatcgacaacatggagtggaaccctgaggtggacgtccacctgaagtcggacggctacaccaacttctccctgaagacgctggactccggcaagcggcagtgcaaggaggccctgcagcgcgagctggggctgcaggtccgcggcgacgtgccgctgctcgggttcatcgggcggctggacgggcagaagggcgtggagatcatcgcggacgcgatgccctggatcgtgagccaggacgtgcagctggtgatgctgggcacggggcgccacgacctggagagcatgctgcagcacttcgagcgggagcaccacgacaaggtgcgcgggtgggtggggttctccgtgcgcctggcgcaccggatcacggcgggcgccgacgcgctcctcatgccctcccggttcgagccgtgcgggctgaaccagctctacgcgatggcctacggcaccatccctgtcgtgcacgccgtcggcggcttgagggataccgtgccgccgttcgaccccttcaaccactccgggctcgggtggacgttcgaccgcgccgaggcgcacaagctgatcgaggcgctcgggcactgcctccgcacctaccgggaccacaaggagagctggaggggcctccaggagcgcggcatgtcgcaggacttcagctgggaacatgccgccaagctctacgaggacgtcctcgtccaggccaagtaccagtggtgaacgctgctacccggtccagccccgcatgcgtgcatgagaggatggaaatgcgcattgcgcacttgcagatttggcgcacgcaggaacgtgccgtccttcttgatgagaacgccggcatccgcgaggttgagacgctgattccgatctggtccgtcgcagagtagagtgaaacgctccttgttgcaggtatatgggaatgttttttttcctttttttttgcgagggaggtatatgggaatgttaacttggtattgtaatgtggtatgctgtgtgcattattacatcggttgttgttgcttattcttgctagctaagtcggaggccaagagcgaaagctagctcacatgtctgatgtatgcaagtgacatggttggtttggttgtgcagtgcaaacggcaggaatgggaagtgaattcctccctgcttaaattaaaaaaaaaaaaaaaaaaa

SEQ ID NO: 2 - protein, 802 aa, Hordeum vulgare
MSSAVASPASFLALASASPGRSSRRRARVGASPTRAGAGRLQWRPSPLQRTARDGAVAARAAGIDDAAPGRQPRARRYGAATKVADPVKTLDRDAAEGGGPSPPAPRQDAARLPSKNGTLINGENKPTGGGGATKDSGLPTPARAPHLSIQNRVPVNGENKHKVASPPTSIVDVASPGSAANISISNKVPPSVVPAKKTPPSSVFPAKKTLPSSGSNFVSSASAPRLDTVSDVELAQKKDALIVKEAPKPKALSAPAAPAVQEDLWDFKKYIGFEEPVEAKDDGSAVADDAGSFEHHQNHDSGPLAGENVMNVVVVAAECSPWCKTGGLGDVAGALPKALAKRGHRVMVVVPRYGDYEEAYDVGVRKYYKAAGQDMEVNYFHAYIDGVDFVFIDAPLFRHRQQDIYGGSRQEIMKRMILFCKAAVEVPWHVPCGGVPYGDGNLVFIANDWHTALLPVYLKAYYRDHGLMQYSRSVMVIHNIAHQGRGPVDEFPFTELPEHYLEHFRLYDPVGGEHANYFAAGLKMADQVVVVSPGYLWELKTVEGGWGLHDIIRQNDWKTRGIVNGIDNMEWNPEVDVHLKSDGYTNFSLKTLDSGKRQCKEALQRELGLQVRGDVPLLGFIGRLDGQKGVEIIADAMPWIVSQDVQLVMLGTGRHDLESMLQHFEREHHDKVRGWVGFSVRLAHRITAGADALLMPSRFEPCGLNQLYAMAYGTIPVVHAVGGLRDTVPPFDPFNHSGLGWTFDRAEAHKLIEALGHCLRTYRDHKESWRGLQERGMSQDFSWEHAAKLYEDVLVQAKYQW

SEQ ID NO: 3 - DNA, 2842 bp, Triticum aestivum
  source 1..2842: mol_type = other DNA
gctgccaccacctccgcctgcgccgcgctctgggcggaggaccaacccgcgcatcgtaccatcgcccgccccgatcccggccgccgccatgtcgtcggcggtcgcgtccgccgcgtccttcctcgcgctcgcctccgcctcccccgggagatcacgcaggcgggcgagggtgagcgcgccgccaccccacgccggggccggcaggctgcactggccgccgtggccgccgcagcgcacggctcgcgacggaggtgtggccgcgcgcgccgccgggaagaaggacgcgagggtcgacgacgacgccgcgtccgcgaggcagccccgcgcacgccgcggtggcgccgccaccaaggtcgcggagcggagggatcccgtcaagacgctcgatcgcgacgccgcggaaggtggcgcgccggcaccgccggcaccgaggcaggacgccgcccgtccaccgagtatgaacggcacgccggtgaacggtgagaacaaatctaccggcggcggcggcgcgaccaaagacagcgggctgcccgcacccgcacgcgcgccccatccgtcgacccagaacagagtaccagtgaacggtgaaaacaaagctaacgtcgcctcgccgccgacgagcatagccgaggtcgtggctccggattccgcagctaccatttccatcagtgacaaggcgccggagtccgttgtcccagccgagaagccgccgccgtcgtccggctcaaatttcgtggtctcggcttctgctcccaggctggacattgacagcgatgttgaacctgaactgaagaagggtgcggtcatcgtcgaagaagctccaaacccaaaggctctttcgccgcctgcagcccccgctgtacaagaagacctttgggacttcaagaaatacattggcttcgaggagcccgtggaggccaaggatgatggctgggctgttgcagatgatgcgggctcctttgaacatcaccagaaccatgattccggacctttggcaggggagaacgtcatgaacgtggtcgtcgtggctgctgaatgttctccctggtgcaaaacaggtggtcttggagatgttgccggtgctttgcccaaggctttggcgaagagaggacatcgtgttatggttgtggtaccaaggtatggggactatgaggaagcctacgatgtcggagtccgaaaatactacaaggctgctggacaggatatggaagtgaattatttccatgcttatatcgatggagttgattttgtgttcattgacgctcctctcttccgacaccgccaggaagacatttatgggggcagcagacaggaaattatgaagcgcatgattttgttctgcaaggccgctgtcgaggttccttggcacgttccatgcggcggtgtcccttatggggatggaaatctggtgtttattgcaaatgattggcacacggcactcctgcctgtctatctgaaagcatattacagggaccatggtttgatgcagtacactcggtccattatggtgatacataacatcgcgcaccagggccgtggcccagtagatgaattcccgttcaccgagttgcctgagcactacctggaacacttcagactgtacgaccccgtgggtggtgagcacgccaactacttcgccgccggcctgaagatggcggaccaggttgtcgtggtgagccccgggtacctgtgggagctcaagacggtggagggcggctgggggcttcacgacatcatacggcagaacgactggaagacccgcggcatcgtcaacggcatcgacaacatggagtggaaccccgaggtggacgtccacctcaagtcggacggctacaccaacttctccctggggacgctggactccggcaagcggcagtgcaaggaggccctgcagcgcgagctgggcctgcaggtccgcgccgacgtgccgctgctcggcttcatcggccgcctggacgggcagaagggcgtggagatcatcgcggacgccatgccctggatcgtgagccaggacgtgcagctggtcatgctgggcaccggccgccacgacctggagagcatgctgcggcacttcgagcgggagcaccacgacaaggtgcgcgggtgggtggggttctccgtgcgcctggcgcaccggatcacggcgggcgccgacgcgctcctcatgccctcccggttcgagccgtgcgggttgaaccagctttacgccatggcctacggcaccgtccccgtcgtgcacgccgtcggcggggtgagggacaccgtgccgccgttcgaccccttcaaccactccggcctcgggtggacgttcgaccgcgccgaggcgcacaagctgatcgaggcgctcgggcactgcctccgcacctaccgggactacaaggagagctggaggggcctccaggagcgcggcatgtcgcaggacttcagctgggagcatgccgccaagctctacgaggacgtcctcctcaaggccaagtaccagtggtgaacgctagctgctagccgctccagccccgcatgcgtgcatgcatgagagggtggaactgcgcattgcgcccgcaggaacgtgccatccttctcgatgggagcgccggcatccgcgaggtgcagtgacatgagaggtgtgtgtggttgagacgctgattccgatctcgatctggtccgtagcagagtagagcggacgtagggaagcgctccttgttgcaggtatatgggaatgttgtcaacttggtattgtagtttgctatgttgtatgcgttattacaatgttgttacttattcttgttaagtcggaggcaaagggcgaaagctagctcacatgaaaaaaaaaaaaaaaaaaaaa

SEQ ID NO: 4 - protein, 799 aa, Triticum aestivum
MSSAVASAASFLALASASPGRSRRRARVSAPPPHAGAGRLHWPPWPPQRTARDGGVAARAAGKKDARVDDDAASARQPRARRGGAATKVAERRDPVKTLDRDAAEGGAPAPPAPRQDAARPPSMNGTPVNGENKSTGGGGATKDSGLPAPARAPHPSTQNRVPVNGENKANVASPPTSIAEVVAPDSAATISISDKAPESVVPAEKPPPSSGSNFVVSASAPRLDIDSDVEPELKKGAVIVEEAPNPKALSPPAAPAVQEDLWDFKKYIGFEEPVEAKDDGWAVADDAGSFEHHQNHDSGPLAGENVMNVVVVAAECSPWCKTGGLGDVAGALPKALAKRGHRVMVVVPRYGDYEEAYDVGVRKYYKAAGQDMEVNYFHAYIDGVDFVFIDAPLFRHRQEDIYGGSRQEIMKRMILFCKAAVEVPWHVPCGGVPYGDGNLVFIANDWHTALLPVYLKAYYRDHGLMQYTRSIMVIHNIAHQGRGPVDEFPFTELPEHYLEHFRLYDPVGGEHANYFAAGLKMADQVVVVSPGYLWELKTVEGGWGLHDIIRQNDWKTRGIVNGIDNMEWNPEVDVHLKSDGYTNFSLGTLDSGKRQCKEALQRELGLQVRADVPLLGFIGRLDGQKGVEIIADAMPWIVSQDVQLVMLGTGRHDLESMLRHFEREHHDKVRGWVGFSVRLAHRITAGADALLMPSRFEPCGLNQLYAMAYGTVPVVHAVGGVRDTVPPFDPFNHSGLGWTFDRAEAHKLIEALGHCLRTYRDYKESWRGLQERGMSQDFSWEHAAKLYEDVLLKAKYQW

SEQ ID NO: 5 - DNA, 2780 bp, Triticum aestivum
  source 1..2780: mol_type = other DNA
cggaggaccaacccgcgcatcgtaccatcgcccgccccgatcccggccgccgccatgtcgtcggcggtcgcgtccgccgcgtccttcctcgcgctcgcctccgcctcccccgggagatcacgcaggcgggcgagggtgagcgcgccgccaccccacgccggggccggcaggctgcactggccgccgtggccgccgcagcgcacggctcgcgacggaggtgtggccgcgcgcgccgccgggaagaaggacgcgagggtcgacgacgacgccgcgtccgcgaggcagccccgcgcacgccgcggtggcgccgccaccaaggtcgcggagcggagggatcccgtcaagacgctcgatcgcgacgccgcggaaggtggcgcgccggcaccgccggcaccgaggcaggacgccgcccgtccaccgagtatgaacggcacgccggtgaacggtgagaacaaatctaccggcggcggcggcgcgaccaaagacagcgggctgcccgcacccgcacgcgcgccccatccgtcgacccagaacagagtaccagtgaacggtgaaaacaaagctaacgtcgcctcgccgccgacgagcatagccgaggtcgtggctccggattccgcagctaccatttccatcagtgacaaggcgccggagtccgttgtcccagccgagaagccgccgccgtcgtccggctcaaatttcgtggtctcggcttctgctcccaggctggacattgacagcgatgttgaacctgaactgaagaagggtgcggtcatcgtcgaagaagctccaaacccaaaggctctttcgccgcctgcagcccccgctgtacaagaagacctttgggacttcaagaaatacattggcttcgaggagcccgtggaggccaaggatgatggctgggctgttgcagatgatgcgggctcctttgaacatcaccagaaccatgattccggacctttggcaggggagaacgtcatgaacgtggtcgtcgtggctgctgaatgttctccctggtgcaaaacaggtggtcttggagatgttgcgggtgctctgcccaaggctttggcaaagagaggacatcgtgttatggttgtggtaccaaggtatggggactatgaggaagcctacgatgtcggagtccgaaaatactacaaggctgctggacaggatatggaagtgaattatttccatgcttatatcgatggagttgattttgtgttcattgacgctcctatcttccgacaccgtcaggaagacatttatgggggcagcagacaggaaattatgaagcgcatgattttgttctgcaaggccgctgtcgaggttccttggcacgttccatgcggcggtgtcccttatggggatggaaatctggtgtttattgcaaatgattggcacacggcactcctgcctgtctatctgaaagcatattacagggaccatggtttgatgcagtacactcggtccattatggtgatacataacatcgcgcaccagggccgtggcccagtagatgaattcccgttcaccgagttgcctgagcactacctggaacacttcagactgtacgaccccgtgggtggtgagcacgccaactacttcgccgccggcctgaagatggcggaccaggttgtcgtggtgagccccgggtacctgtgggagctcaagacggtggagggcggctgggggcttcacgacatcatacggcagaacgactggaagacccgcggcatcgtcaacggcatcgacaacatggagtggaaccccgaggtggacgtccacctccagtcggacggctacaccaacttctccctgagcacgctggactccggcaagcggcagtgcaaggaggccctgcagcgcgagctgggcctgcaggtccgcgccgacgtgccgctgctcggcttcatcggccgcctggacgggcagaagggcgtggagatcatcgcggacgccatgccctggatcgtgagccaggacgtgcagctggtcatgctgggcaccggccgccacgacctggagagcatgctgcggcacttcgagcgggagcaccacgacaaggtgcgcgggtgggtggggttctccgtgcgcctggcgcaccggatcacggcgggcgccgacgcgctcctcatgccctcccggttcgagccgtgcgggctgaaccagctctacgccatggcctacggcaccgtccccgtcgtgcacgccgtcggcgggctgagggacaccgtgccgccgttcgaccccttcaaccactccggcctcgggtggacgttcgaccgcgccgaggcgcacaagctgatcgaggcgctcgggcactgcctccgcacctaccgggactacaaggagagctggaggggcctccaggagcgcggcatgtcgcaggacttcagctgggagcatgccgccaagctctacgaggacgtcctcctcaaggccaagtaccagtggtgaacgctagctgctagccgctccagccccgcatgcgtgcatgcatgagagggtggaactgcgcattgcgcccgcaggaacgtgccatccttctcgatgggagcgccggcatccgcgaggtgcagtgacatgagaggtgtgtgtggttgagacgctgattccgatctcgatctggtccgtagcagagtagagcggacgtagggaagcgctccttgttgcaggtatatgggaatgttgtcaacttggtattgtagtttgctatgttgtatgcgttattacaatgttgttacttattcttgttaaaaaaaaaaaaaaaaaaaaaaaaaaaa

SEQ ID NO: 6 - protein, 799 aa, Triticum aestivum
MSSAVASAASFLALASASPGRSRRRARVSAPPPHAGAGRLHWPPWPPQRTARDGGVAARAAGKKDARVDDDAASARQPRARRGGAATKVAERRDPVKTLDRDAAEGGAPAPPAPRQDAARPPSMNGTPVNGENKSTGGGGATKDSGLPAPARAPHPSTQNRVPVNGENKANVASPPTSIAEVVAPDSAATISISDKAPESVVPAEKPPPSSGSNFVVSASAPRLDIDSDVEPELKKGAVIVEEAPNPKALSPPAAPAVQEDLWDFKKYIGFEEPVEAKDDGWAVADDAGSFEHHQNHDSGPLAGENVMNVVVVAAECSPWCKTGGLGDVAGALPKALAKRGHRVMVVVPRYGDYEEAYDVGVRKYYKAAGQDMEVNYFHAYIDGVDFVFIDAPIFRHRQEDIYGGSRQEIMKRMILFCKAAVEVPWHVPCGGVPYGDGNLVFIANDWHTALLPVYLKAYYRDHGLMQYTRSIMVIHNIAHQGRGPVDEFPFTELPEHYLEHFRLYDPVGGEHANYFAAGLKMADQVVVVSPGYLWELKTVEGGWGLHDIIRQNDWKTRGIVNGIDNMEWNPEVDVHLQSDGYTNFSLSTLDSGKRQCKEALQRELGLQVRADVPLLGFIGRLDGQKGVEIIADAMPWIVSQDVQLVMLGTGRHDLESMLRHFEREHHDKVRGWVGFSVRLAHRITAGADALLMPSRFEPCGLNQLYAMAYGTVPVVHAVGGLRDTVPPFDPFNHSGLGWTFDRAEAHKLIEALGHCLRTYRDYKESWRGLQERGMSQDFSWEHAAKLYEDVLLKAKYQW

SEQ ID NO: 7 - DNA, 6811 bp, Triticum aestivum
  source 1..6811: mol_type = genomic DNA
gggggccgttcgtacgtacccacccctcgtgtaaagccgccgccgtcgtcgccgtcccccgctcgcggccatttcctcggcctgaccccgtgcgtttaccccacacagagcacactccagtccagtccagcccactgccgcgctactccccactcccactgccaccacctccgcctgcgccgcgctctgggcggaccaacccgcgcatcgtatcacgatcacccaccccgatcccggccgccgccatgtcgtcggcggtcgcgtccgccgcgtccttcctcgcgctcgcgtccgcctcccccgggagatcacggaggaggacgagggtgagcgcgtcgccaccccacaccggggctggcaggttgcactggccgccgtcgccgccgcagcgcacggctcgcgacggagcagtggccgcgcgcgccgccgggaagaaggacgcggggatcgacgacgccgcgcccgcgaggcagccccgcgcactccgcggtggcgccgccaccaaggtagttagttatgaccaagttatgacgcgtgcgcgcgccttgagatcatcgtcgtctcgctgacaaattgtttatacaaaacgcacgcgcgcgcgtgtgtgcaggttgcggagcggagggatcccgtcaagacgctcgatcgcgacgccgcggaaggtggcgcgccgtccccgccggcaccgaggcaggaggacgcccgtctgccgagcatgaacggcatgccggtgaacggtgaaaacaaatctaccggcggcggcggcgcgactaaagacagcgggctgcccgcacccgcacgcgcgccccagccgtcgagccagaacagagtaccggtgaatggtgaaaacaaagctaacgtcgcctcgccgccgacgagcatagccgaggtcgcggctccggatcccgcagctaccatttccatcagtgacaaggcgccagagtccgttgtcccagccgagaaggcgccgccgtcgtccggctcaaatttcgtgccctcggcttctgctcccgggtctgacactgtcagcgacgtggaacttgaactgaagaagggtgcggtcattgtcaaagaagctccaaacccaaaggctctttcgccgcctgcagcacccgctgtacaacaagacctttgggacttcaagaaatacattggtttcgaggagcccgtggaggccaaggatgatggccgggctgttgcagatgatgcgggctccttcgaacaccaccagaatcacgattccgggcctttggcaggggagaacgtcatgaacgtggtcgtcgtggctgctgaatgttctccctggtgcaaaacaggcatggacattacctcttcagtgtcttttttctctctgttcataaaacctggcttgaattactcataagaacaaacgttgtgttgcataggtggtcttggagatgttgccggtgctttgcccaaggctttggcgaagagaggacatcgtgttatggtaccacatgctttcatttaactctgttgaatccatatgttcgaataatatcagtgagcagtataatgttattaagtgcaagacatgaaagtgttcttctgttatagagtatttcatagccaaccctggaggttaggttgttggggcctactgggtgtgggagggggtttgaaaaaagtgttggttagcagtcggatttcacaaagaacgctgataaccacgccatcagtgaagggaatgaatgtcgggtacccgatcgaccgttttgcccgacgtcaggtttacccgccctgtaggtccgaataagtagttcctatcctcagttaagtaccaaatatcggcagcgcccgtgtgtgtatttatagtactggatgatcaatttatcaacattttcagttaatggttgctatcatattcactgtaattgttagtaaacagtggatgtttgtaatgtagatgatggctaaatgtatgttgtcaagctttcatttcaatgcaatttttattgggagctagttttggggttcggttagagccaccaaaaccccagaatttctgggagttggcttgtgagagagggttttggcgagttgactttcgggattcagttagagacgctcttactagttccagtaaagaagtaaactattttctgcagacatcccaattattctgtagaaattagaagtagaaaatagttatggtatcatataaccatatattattcaaaatctagaatcatggacttggctagactttgatgatctgaaattttaaatttgatgataattgagaaatgatcctttctatcttaggttgtggtaccaaggtatggggactatgaggaagcctacgatgtcggagtccgaaaatactacaaggctgctggacaggtaagcgaaaatgcaatcaaaggggagctgaaatttcaatgcttactatcataataaatcaattttaagtgtttttttttgtcctgcaggatatggaagtgaattatttccatgcttatatcgatggagttgattttgtgttcattgacgctcctctcttccgacaccgccaggaagacatttatgggggcagcagacaggttaatcttctatatgttggtgtttgattgcactgataaactgagaacaagccaaggcctactgaccggcatatgattacacattttattttttcaggaaattatgaagcgcatgattttgttctgcaaggccgctgtcgaggtatcctctccaactcaattgacaacctattacaactatacaattatgtgtatgcatgtatttcaacagatacgtaatctcttgtgaagtgcatatatactaacaacatttcaataccttacatgcacatttggtcaagcgttatgatttaacttctaataatctattgcactgatgaacaattatcttgatgatccttggtacttcatcgttatgtttccatgttctcttcaccgattgatttggaaatagcatttccacctgccacaaacaataatatacactcctactttcatccaatgtagatattttcgcacttggcatatcatcccattaaatattattggtccatcatttttattcctctataatttgcaggttccatggcacgttccatgcggcggtgtcccttatggggatggaaatctggtgtttattgcaaatgattggcacacggcactcctgcctgtctatctgaaagcatattacagggaccatggtttgatgcagtacactcggtccattatggtgatacataacatcgctcaccaggttccttttctccaaatcttgatttttctctagtctctactatttactccacattgtttgaggaaactaaacgggttgcaaaattatgatggcttatgaaagttatagtcttatataggtaaatgcaccagtggtgcttgcacttgtcacgcgtgttcactttggtgcttacagttgtagacaataaaaaactagtgcaaaaacttggctgttgtgtgcaatacggtgcattttccgtatgtaggagtcaaatattgcctgtgtggcattgtattcccgtctatagctgttagaccatgcctacgtcgccattgggcccacacaccctctattacatgtgggccccacttgtcagcctatgacataaataaatggaaatttataatgaaaatgatggcctggggtcttgaaaatgggccgtcgcaggtatgctggtagccagcatgccctaatcattaatccccctatgcacttcatgtcttgtgtatgtgtgtgtgggaaagggggggggtatgtatgcttatgcttatatcctttgctccaaggctgccatcctcaacaagcccacctccagcttcaacacggccagcgccttcatgatggcccaggtgctccgcaccatcgatcgaagcggcaacgtcgtcgtcacgaccatccaccaacccaacgcacaaaatcctcaggctccgttcggtacggaggaagagaaaatgcaggaaaaaacaacttcatgggaacaaggtttggtgaacagtaaaaacatgtggaatctgaaaatgtaggtaccagaaaaaccggcctgttcggtttgcaggaaaaagcatacttgcagcagcactgtttggccctttccagtgtaaggctaaccatagtgggagtaacataactaatattatgtacttggaactcacaaacatgcttatgtggcaggcaattaaagaagagagagagagtcatagtaacatagttagataccgtatcataataaatattatgttactatgtgtcatgcatgacaataaatgagaccatctgtgatactacgttatgatattatgcactatagatgtagtatcatacactagtatcatatgcatgatactagtgtatgttactccccactatgaccagcctaacgaggggatgggcatcagtagtgattaccagtttttattattttttattcggctggacgccctactcttgtggtgcgtagcggaaaaaggcagtgctagcttcgggactccgcgagcacatgaggttctaccggattttagatttcctataaaaagtacaggctcacgcaacttttcaatggaacagaccatcaagattcctttgaaccgaacgcactgcatgcaagaattccaatgaaaaacgagccgtcaaatgttcctgcgaatttcctctataccgaacagaccctcaacatcctcaaatagtgagcatgcccctcttgtcctttccccctcgtacccaaacgccatttggcgctcttggtgttggatcatttgcctcgggcatttgccaatttggcgccgaaccatattaaagctatgactacttggtgttggatcagggacgcaaagaatcccataaatattgcaacgttcattcaaattcttaacatttgcgaggcgcttcatgatttccatctccgtcaggtctgagacatttggtcgtgtacactaaatttctcaggtcacttctcgtctaaatccgcatatgtagctcacttcaatgacttgcctttggtctagctaacgccatttggcgctcttgggcccccttgcttagcaaatttttcatatggctcgcactgcgcaagaggatttagatcacgggcagacgcgctagacgaggtcttccgcacaatgaacattgcgttctttgctctgctcttcccggagacacttgtgatcttattacgagttgtgccatttcaaacatctgtctctccatggtcgctccagccatagatgccttgttctctgaatggtgggtttcagctaggaacagggtgccaccttcgacaagaagttgcatagtttggtcgtcttgactgcttggtcgatttggaaggaacgcaacataagagtctttgaaggcaaagctaatttctttgatcaagttattagccagatcaaatgtgatgtattctactggtacaaggccggggctatttgctttgagtcactttttagctaaggccgcttgggctaagcgcttggggtcgtttttgctcaactgtagcctgaactttctggactttgtaattttttttatcctctataatgatcacatacagctctcctgcatggttcgaaaaggaaaaatgtgaacatgtgtggcaagtttaagcacaacccgtgcatttacctcaaagttatacaacactgacatgctgaattacattttttttgaggatctgacatgccgaattacatgctttggacagttattcatttcttcggtacaccattggctaattatttctcttgacagttgctgaattagtacatgctttggtcgcagttattcctttgttcggtactctgttgggctaattatttctcttgattgatgttgcatgcagggccgtggcccagtagatgagttcccgttcaccgagttgcctgagcactacctggaacacttcagactgtacgaccccgtgggtggtgaacacgccaactacttcgccgccggcctgaagatggcggaccaggttgtcgtcgtgagcccggggtacctgtgggagctgaagacggtggagggcggctgggggcttcacgacatcatacggcagaacgactggaagacccgcggcatcgtgaacggcatcgacaacatggagtggaaccccgaggtggacgtccacctcaagtcggacggctacaccaacttctccctggggacgctggactccggcaagcggcagtgcaaggaggccctgcagcgggagctgggcctgcaggtccgcggcgacgtgccgctgctcggcttcatcgggcgcctggacgggcagaagggcgtggagatcatcgcggacgcgatgccctggatcgtgagccaggacgtgcagctggtcatgctgggcaccgggcgccacgacctggagggcatgctgcggcacttcgagcgggagcaccacgacaaggtgcgcgggtgggtggggttctccgtgcggctggcgcaccggatcacggccggcgccgacgcgctcctcatgccctcccggttcgagccgtgcggactgaaccagctctacgccatggcctacggcaccgtccccgtcgtgcatgccgtcggcggcctgagggacaccgtgccgccgttcgaccccttcaaccactccgggctcgggtggacgttcgaccgcgcagaggcgcagaagctgatcgaggcgctcgggcactgcctccgcacctaccgggactacaaggagagctggagggggctccaggagcgcggcatgtcgcaggacttcagctgggagcatgccgccaagctctacgaggacgtcctcgtcaaggccaagtaccagtggtga

SEQ ID NO: 8 - protein, 798 aa, Triticum aestivum
MSSAVASAASFLALASASPGRSRRRTRVSASPPHTGAGRLHWPPSPPQRTARDGAVAARAAGKKDAGIDDAAPARQPRALRGGAATKVAERRDPVKTLDRDAAEGGAPSPPAPRQEDARLPSMNGMPVNGENKSTGGGGATKDSGLPAPARAPQPSSQNRVPVNGENKANVASPPTSIAEVAAPDPAATISISDKAPESVVPAEKAPPSSGSNFVPSASAPGSDTVSDVELELKKGAVIVKEAPNPKALSPPAAPAVQQDLWDFKKYIGFEEPVEAKDDGRAVADDAGSFEHHQNHDSGPLAGENVMNVVVVAAECSPWCKTGGLGDVAGALPKALAKRGHRVMVVVPRYGDYEEAYDVGVRKYYKAAGQDMEVNYFHAYIDGVDFVFIDAPLFRHRQEDIYGGSRQEIMKRMILFCKAAVEVPWHVPCGGVPYGDGNLVFIANDWHTALLPVYLKAYYRDHGLMQYTRSIMVIHNIAHQGRGPVDEFPFTELPEHYLEHFRLYDPVGGEHANYFAAGLKMADQVVVVSPGYLWELKTVEGGWGLHDIIRQNDWKTRGIVNGIDNMEWNPEVDVHLKSDGYTNFSLGTLDSGKRQCKEALQRELGLQVRGDVPLLGFIGRLDGQKGVEIIADAMPWIVSQDVQLVMLGTGRHDLEGMLRHFEREHHDKVRGWVGFSVRLAHRITAGADALLMPSRFEPCGLNQLYAMAYGTVPVVHAVGGLRDTVPPFDPFNHSGLGWTFDRAEAQKLIEALGHCLRTYRDYKESWRGLQERGMSQDFSWEHAAKLYEDVLVKAKYQW

SEQ ID NO: 9 - protein, 799 aa, Triticum aestivum
MSSAVASAASFLALASASPGRSRRRARVSAQPPHAGAGRLHWPPWPPQRTARDGAVAALAAGKKDAGIDDAAASVRQPRALRGGAATKVAERRDPVKTLDRDAAEGGGPSPPAARQDAARPPSMNGMPVNGENKSTGGGGATKDSGLPTPARAPHPSTQNRAPVNGENKANVASPPTSIAEAAASDSAATISISDKAPESVVPAEKTPPSSGSNFESSASAPGSDTVSDVEQELKKGAVVVEEAPKPKALSPPAAPAVQEDLWDFKKYIGFEEPVEAKDDGRAVADDAGSFEHHQNHDSGPLAGENVMNVVVVAAECSPWCKTGGLGDVAGALPKALAKRGHRVMVVVPRYGDYEEAYDVGVRKYYKAAGQDMEVNYFHAYIDGVDFVFIDAPLFRHRQEDIYGGSRQEIMKRMILFCKAAVEVPWHVPCGGVPYGDGNLVFIANDWHTALLPVYLKAYYRDHGLMQYTRSIMVIHNIAHQGRGPVDEFPFTELPEHYLEHFRLYDPVGGEHANYFAAGLKMADQVVVVSPGYLWELKTVEGGWGLHDIIRQNDWKTRGIVNGIDNMEWNPEVDAHLKSDGYTNFSLRTLDSGKRQCKEALQRELGLQVRADVPLLGFIGRLDGQKGVEIIADAMPWIVSQDVQLVMLGTGRHDLESMLQHFEREHHDKVRGWVGFSVRLAHRITAGADALLMPSRFEPCGLNQLYAMAYGTVPVVHAVGGLRDTVPPFDPFNHSGLGWTFDRAEAHKLIEALGHCLRTYRDFKESWRALQERGMSQDFSWEHAAKLYEDVLVKAKYQW

SEQ ID NO: 10 - DNA, 2400 bp, Sorghum bicolor
  source 1..2400: mol_type = other DNA
atcgatcgaccaccgaatcccgccgcggccatgtcgtcggcggccgtgtcgtccgcttcctccaccttcttcctcgcgctcgcctcctcggcctccccggggggacgcaggcgggcaagggtcggctcgccgccgttccacaccggcgccggtccgaatttcgcgttctggcgggcgccgccgcgcgtgccccgtgacgcggcgctggtgcgcgcggaggccgaaggcgggggcaaggacgcgccgccggagaggagtggcgacgccgccgccgccgccgccagcaggtggccccgcgccgctcggcgcaagacggtctccaaacggagggatcctcttcagccggtcggccgttacggctcatccggaacggggaacacggccaggagcggcgccgcgaccgcgtccacccagaacgccgcgttggcggccgttgagatcaagtccatcgtcgccgcgccgccgacgagcatagtgaagttcccagccccgggatacagggtgatccttccctctggggacatagcgccggagactgtcgtcccagccccgaagccactgccactgcatggatcgcctggggttgatggagattattcaaatggggttgcacctcctccagctgagccagtagtacaggaggccacttgggatttcaagaaatacatcggttttgaggagcctgtggaagccaaggatgattccggggttgctgcagatggtgctgattcttttgaacactacgagaacaatgattctgggcctttggccggggagaatgtcatgaacgtcatcgtggtggctgctgaatgttctccatggtgcaaaacaggtggccttggagatgttgtgggagctttacccaaggctttagcgagaagaggacatcgtgttatggttgtggttccaaggtatggggactatgtggaagccttcgatatgggaatccggaaatactacaaagctgcaggacaggacttggaagtgaactatttccatgcatttatcgatggagtagattttgtgttcattgatgcacctctcttccggcaccgtcaaaatgacatatacgggggaagtagacaggaaattttgaagcgcatgattttgttctgcaaggttgctgttgaggttccttggcatgttccatgtggcggtgtgtgctacggagatggaaatttggtgttcattgccaatgattggcacactgcactcctgcctgtttatctgaaggcatattacacagaccatgggttaatgcagtacactcgctccattcttgtcatacataacatcgctcaccagggtcgtggtcctgtagctgaattcccgtacatggacttgcctgaacactacctccaacatttcgagctgtacgaccccgtcggtggtgagcacgccaacatctttgccgcgggtctgaagatggcagaccgggtggtgacggtcagccggggctacctgtgggagctgaagacagtggaaggtggctggggcctccacgacatcatccgttccaacgactggaagatcaatggcatcgtgaacggcatcgaccaccaggagtggaaccccaaggtggacgtgcacctgcggtcggacgggtacaccaactactccctcgagacactggacgccggaaagcggcagtgcaaggcggccctgcagcgggagctgggcctggaagtgcgcgacgacgtgccgctgctcggcttcatcgggcgcctggacgggcagaagggcgtggacatcatcggggacgcgatgccgtggatcgcggggcaggacgtgcagctggtgatgctgggcaccgggcgcgccgacctggagcggatgctgcagcacctggagcgggagcacccaagcaaggtgcgcgggtgggtggggttctcggtgcccatggcgcaccgcatcaccgcgggcgccgacgtgctggtgatgccctcccggttcgagccatgcgggctgaaccagctctacgccatggcgtacggcaccgtccccgtggtgcacgccgtcggcgggctcagggacaccgtggcgccgttcgacccgttcggcgacgccgggctcggctggactttcgaccgcgccgaggtcaacaagctgatcgaggcgctcaggcactgcctcgacacgtaccggaactacggggagagctggaagagcatccaggcgcgcggcatggcacaggacctcagctgggaccacgcggcggagctctacgaggacgtcctagtcaaggccaagtaccagtggtgaaccctccggcctccgcatcgctcgatatgtcgagagcaacttctgctcgtcgtgtggttttacaattttcggtttgatccattgtacattgcgcattcgacggtctcggtgaagaacttcatatgcagtgccgcgccg

SEQ ID NO: 11 - protein, 743 aa, Sorghum bicolor
MSSAAVSSASSTFFLALASSASPGGRRRARVGSPPFHTGAGPNFAFWRAPPRVPRDAALVRAEAEGGGKDAPPERSGDAAAAAASRWPRAARRKTVSKRRDPLQPVGRYGSSGTGNTARSGAATASTQNAALAAVEIKSIVAAPPTSIVKFPAPGYRVILPSGDIAPETVVPAPKPLPLHGSPGVDGDYSNGVAPPPAEPVVQEATWDFKKYIGFEEPVEAKDDSGVAADGADSFEHYENNDSGPLAGENVMNVIVVAAECSPWCKTGGLGDVVGALPKALARRGHRVMVVVPRYGDYVEAFDMGIRKYYKAAGQDLEVNYFHAFIDGVDFVFIDAPLFRHRQNDIYGGSRQEILKRMILFCKVAVEVPWHVPCGGVCYGDGNLVFIANDWHTALLPVYLKAYYTDHGLMQYTRSILVIHNIAHQGRGPVAEFPYMDLPEHYLQHFELYDPVGGEHANIFAAGLKMADRVVTVSRGYLWELKTVEGGWGLHDIIRSNDWKINGIVNGIDHQEWNPKVDVHLRSDGYTNYSLETLDAGKRQCKAALQRELGLEVRDDVPLLGFIGRLDGQKGVDIIGDAMPWIAGQDVQLVMLGTGRADLERMLQHLEREHPSKVRGWVGFSVPMAHRITAGADVLVMPSRFEPCGLNQLYAMAYGTVPVVHAVGGLRDTVAPFDPFGDAGLGWTFDRAEVNKLIEALRHCLDTYRNYGESWKSIQARGMAQDLSWDHAAELYEDVLVKAKYQW

SEQ ID NO: 12 - DNA, 2433 bp, Oryza sativa
  source 1..2433: mol_type = other DNA
atgtcgtcggccgtcgtcgcgtcatccaccacgttcctcgtcgcgctcgcctcttcggcgtcacggggagggcccaggagggggagggtggtgggcgtggccgcgccgccggccctgctttacgacggccgcgccggaaggctagccctacgggcgccgcctccgccccggcctcggcctcggcgtcgggatgcgggcgtggtgcgccgcgcggatgatggggagaacgaggcggcggtggagcgggcgggtgaggacgacgaggaggaggaggagttctcttcgggcgcgtggcagccgccccgctcgcgtcgcggcggcgtcggcaaggtcttgaagcggaggggcaccgtcccgcccgtcggccggtacggctccggcggtgacgcggcgagagtgcgcggggcggccgcgccggcgccggcgccgacacaggacgccgcctcaagtaagaacggagcgcttctcagcggccgcgacgacgacacacctgcctcacggaacggatcggtcgttaccggcgccgacaagcctgccgccgccacgccgccggtgaccataacgaagctcccagcgccggactcccccgtgatccttccatccgtagacaagccgcagccggagttcgtcatcccagacgcgacggcgccggcgccgccaccgcccggttcaaatcccaggtcgtccgctcctctccccaagcctgacaattcggaatttgcagaggataagagcgcaaaagttgttgagagtgctccgaagccaaaggcgactagatcttcccctattcctgcggtagaagaggagacgtgggatttcaagaaatattttgatctgaacgaaccggacgccgcggaggatggcgatgacgatgatgactgggctgattcagatgcgtcagattctgagatcgaccaggatgacgattcgggccctttggctggggagaatgtcatgaacgtgatcgtggtggctgctgaatgttctccctggtgcaaaacaggtgggcttggagatgttgcaggtgctttacccaaagctttggcgaggagaggacatcgtgttatggttgtggtaccaaggtacggtgattacgcggaagcccaggatgtaggaatcaggaaatactacaaggctgctggacaggatctggaagtgaaatatttccatgcatttatcgatggagttgattttgtgttcattgacgctcctctcttccgtcaccgtcaggatgacatctatggggggaacagacaggaaatcatgaagcgcatgattctgttttgtaaggctgctgttgaggttccttggcacgttccatgcggtggtgtgccctatggggatggcaacttggtgttccttgcaaacgattggcacactgcactcctgcctgtttatctgaaggcatattacagagacaatggcatgatgcagtacactcgctctgtccttgtgatacataatatcgcttaccagggccgtggcccagtagatgaattcccctacatggaattgccggagcactacctggatcacttcaagctgtacgaccccgtcggcggcgagcacgccaacatcttcggcgcgggcctgaagatggcggaccgggtggtgaccgtgagccccggctacctctgggagctgaagacgacggagggcggctggggcctccacgacatcatacgggagaacgactggaagatgaacggcatcgtgaacggcatcgactaccgggagtggaacccggaggtggacgtgcacctgcagtccgacggctacgccaactacaccgtggcctcgctggactccggcaagccgcggtgcaaggcggcgctgcagcgcgagctggggctggaggtgcgcgacgacgtgccgctgatcgggttcatcgggcggctcgacgggcagaaaggggtggacatcatcggcgacgcgatgccgtggatcgccgggcaggacgtgcagctggtgctgctgggctccggccgccgcgacctggaggtgatgctgcagcggttcgaggcgcagcacaacagcaaggtgcgcgggtgggtggggttctcggtgaagatggcgcaccggatcacggcgggcgccgacgtgctggtcatgccgtcgcggttcgagccgtgcggcctcaaccagctctacgccatggcgtacggcaccgtccccgtcgtgcacgccgtcggcgggctgagggacaccgtgtcggcgttcgacccgttcgaggacaccggcctcgggtggacgttcgaccgcgccgagccgcacaagctcatcgaggcgctcggccactgcctcgagacgtaccgcaagtacaaggagagctggagggggctccaggtgcgcggcatgtcgcaggacctcagctgggaccacgccgccgagctctacgaggaggtccttgtcaaggccaagtaccaatggtga

SEQ ID NO: 13 - protein, 810 aa, Oryza sativa
MSSAVVASSTTFLVALASSASRGGPRRGRVVGVAAPPALLYDGRAGRLALRAPPPPRPRPRRRDAGVVRRADDGENEAAVERAGEDDEEEEEFSSGAWQPPRSRRGGVGKVLKRRGTVPPVGRYGSGGDAARVRGAAAPAPAPTQDAASSKNGALLSGRDDDTPASRNGSVVTGADKPAAATPPVTITKLPAPDSPVILPSVDKPQPEFVIPDATAPAPPPPGSNPRSSAPLPKPDNSEFAEDKSAKVVESAPKPKATRSSPIPAVEEETWDFKKYFDLNEPDAAEDGDDDDDWADSDASDSEIDQDDDSGPLAGENVMNVIVVAAECSPWCKTGGLGDVAGALPKALARRGHRVMVVVPRYGDYAEAQDVGIRKYYKAAGQDLEVKYFHAFIDGVDFVFIDAPLFRHRQDDIYGGNRQEIMKRMILFCKAAVEVPWHVPCGGVPYGDGNLVFLANDWHTALLPVYLKAYYRDNGMMQYTRSVLVIHNIAYQGRGPVDEFPYMELPEHYLDHFKLYDPVGGEHANIFGAGLKMADRVVTVSPGYLWELKTTEGGWGLHDIIRENDWKMNGIVNGIDYREWNPEVDVHLQSDGYANYTVASLDSGKPRCKAALQRELGLEVRDDVPLIGFIGRLDGQKGVDIIGDAMPWIAGQDVQLVLLGSGRRDLEVMLQRFEAQHNSKVRGWVGFSVKMAHRITAGADVLVMPSRFEPCGLNQLYAMAYGTVPVVHAVGGLRDTVSAFDPFEDTGLGWTFDRAEPHKLIEALGHCLETYRKYKESWRGLQVRGMSQDLSWDHAAELYEEVLVKAKYQW

SEQ ID NO: 14 - DNA, 2248 bp, Zea mays
  source 1..2248: mol_type = other DNA
gcacctttgtagccccgcgctcggcgcaatgcggtctccaaacctagggatcctcttcagccggtcggccggtacggctccgcgacgggaaacacggccaggaccggcgccgcttcctgccagaacgccgcattggcggacgttgagatcaagtccatcgtcgccgcgccgccgacgagcatagtgaagttcccagcgccgggctacaggatgatccttccctctggggacatagcgccggagactgtcctcccagccccgaagccactgcatgaatcgcctgcggttgacggagattcaaatggaattgcacctcctacagttgagccattagtacaggaggccacttgggatttcaagaaatacatcggttttgacgagcctgtcgaagcgaaggatgattccagggttggtgcagatgatgctggttcttttgaacattatggggacaatgattctgggcctttggccggggagaatgttatgaacgtgatcgtggtggctgctgaatgttctccatggtgcaaaacaggtggtcttggagatgttgtgggagctttacccaaggctttagcgagaagaggacatcgtgttatggttgtggtaccaaggtatggggactatgtggaagcctttgatatgggaatccggaaatactacaaagctgcaggacaggacctagaagtgaactatttccatgcatttattgatggagtcgactttgtgttcattgatgcccctcttttccggcaccgtcaagatgacatatatggggggaagtaggcaggagatcatgaagcgcatgattttgttttgcaaggttgctgttgaggttccttggcacgttccatgtggtggtgtgtgctacggagatggaaatttggtgttcattgccaatgattggcacactgcactcctgcctgtttatctgaagggcatattacagagaccatgggttaatgcagtacactcgctccgtcctcgtcatacataacatcgcccaccagggccgtggtcctgtagatgaattcccgtacatggacttgcctgaacactaccttcaacatttcgagctgtacgatcccgtgggtggcgagcacgccaacatctttgccgcgggtctgaagatggcagaccgggtggtgactgtcagccgcggctacctgtgggagctgaagacagtggaaggcggctggggcctccacgacatcatccgttctaacgactggaagatcaatggcatcgtgaacggcatcgaccacgaggagtggaaccccaaggtggacgtgcacctgcggtcggacggctacaccaactactccctcgagacactcgacgctggaaagcggcagtgcaaggcggccctgcagcgggagctgggcctggaagtgcgcgacgacgtgccgctgctcggcttcatcgggcgtctggatggacagaagggcgtggacatcatcggggacgcgatgccgtggatcgcggggcaggacgtgcagctggtgatgctgggcaccgggcgcgccgacctggaacgaatgctgcagcacttggagcgggagcatcccaacaaggtgcgcgggtgggtcgggttctcggtgcctatggcgcatcgcatcacggcgggcgccgacgtgctggtgatgccctcccgcttcgagccctgcgggctgaaccagctctacgcgatggcatacggcaccgtccctgtggtgcacgccgtgggcgggctcagggacaccgtggcgccgttcgacccgttcagcgacgccgggctcgggtggacttttgaccgcgccgaggccaacaagctgatcgaggcgctcaggcactgcctcgacacgtaccggaactacgaggatagctggaagagtctccaggcgcgcggcatgtcgcaggacctcagctgggaccacgcggctgagctctacgaggacgtccttgtcaaggccaagtaccagtggtgaaccctccgccctccgcatcaatatcttcggtttgatcccattgtacatcgcgcgtttgacggtctcggtgaagaacttcatatgcagtgacgcgccgctggggttggtagcagtactatgggattgcattgagctgtgtcactatgtgctttcgacaggacagtagtgtaggttgtatgcaagtttagttttttttcattactgatatttggaatgtcaacacaataaatgaagctactatgtgtttcgtaagtataatgttacttatttaggtgataaaaaaa

SEQ ID NO: 15 - DNA, 2645 bp, Oryza sativa
  source 1..2645: mol_type = other DNA
gaatctcttttctctcgtcgtcgtctcgtcttcccacctctcggattcccgagctccacacgacacgacccacctcggccactctccgccgtcggtggccagtggcctccaccccctccgtcccccggggaccacaccggcgtaacatcagccacacctccggcagatctttcccccgccgcttaattcacctcctcttcgccgcggcgcaggcggagcagggtgagcggggtttggtggcacttgtatggtggcacggggctgcggttgcattgggagcggcggggtttggttcgggacggagccgtcgtgtgctcggcgtcggccgccggtggtgaggatggcgtcgcgaaggcgaagacgaagtcagcggggagctcgaaggcggtcgctgtgcagggcagcacggccaaggctgatcatgttgaggattcagtttcatcacccaaatacgtgaaacctgccgttgcgaagcaaaatggagaggttgtgagcagagctaccaaatctgatgcaccagtgtctaaacccaaagttgacccgtcagttcctgcttccaaggctgaagctgatggtaatgcacaagctgtggaatcgaaggctgcactggacaagaaggaagacgtcggtgttgctgaaccgttggaagctaaggctgatgctggtggagatgcaggtgcagtaagctctgcagatgatagtgagaacaaggaatctggtccattggcagggccaaatgtgatgaatgtcattgtggtggcttctgaatgttctcctttctgtaaaacaggtggtcttggagatgttgtgggtgctttgcctaaggctctggcaagaagaggacatcgtgttatggttgtgataccaagatacggagaatatgcagaagccaaggatctaggtgtccgcaaacgttacagggtagctggacaggattcagaagtgagttattttcatgcttttatcgatggtgttgattttgtgtttctagaggcccctccctttcgacaccggcacaatgatatttatggaggagaaagatttgatgttttgaagcgtatgattttgttctgcaaagctgctgttgaggttccttggtttgctccatgtggtggttccatctacggtgatggcaatttagtcttcattgcaaatgattggcatactgcacttttacccgtatatttgaaggcatattaccgagataatggcttgatgcagtatactcgctctgttcttgtgatacacaacattgctcatcagggtcgtggccctgtagatgacttcgccacgatggacttgcccgagcactacatcgatcatttcagactgtacgaccccgtcggcggtgagcacagcaacgtgttcgccgctggcctgaagatggctgaccgcgcggtcaccgtcagccatggctatctgtgggagatcaagacaatggacggtggctggggcctccacgagataataaaccacaacgactggaagctgcagggcatcgtgaacggcatcgacatggccgaatggaaccccgaggtggacgagcacctgcagtccgacggctacgccaactacacgttcgagacgctggacaccggcaagaaacagtgcaaggaggcgctgcagcggcagctgggcctgcaggtccgcgacgacgtgccgctgatcgggttcatcgggcggctcgaccaccagaagggcgtggacatcatcggcgacgcgatgccgtggatcgccgggcaggacgtgcaggtggtgatgggggcacggggcggccggacctggaggagatgctgcggcggttcgagtcggagcacaacgacaaggtgcgcgggtgggtggggttctcggtgcagctggcgcaccggatcacggcgggcgccgacgtcctcctgatgccgtcgcggttcgagccgtgcgggctgaaccagctgtacgccatggcgtacggcaccgtgcccgtggtgcacgccgtgggcgggctccgcgacacggtggcgccgttcgacccgttcgccgacacggggctcgggtggacgttcgaccgcgccgaggcgaaccggatgatcgacgcgctcgggcactgcctcaacacgtaccggaactacaaggagagctggagggggctccaggcgcgcggcatggcgcaggacctcagctgggaccacgccgccgagctctacgaggacgtcctcgtcaaggccaagtaccagtggtgagctggtggtatttttcgcgacgcctctggatcccgccgtggatgggcgcgctttttctctagggcggctctgcgtcggagacgctcagacgcatcgcgcgcgttcgcgcccacacgatgactggcacgctgctgctgagacggcggcacgcgatgccatcatgccaccggacggtaggagcaacggtggaagtgaactgtactactggtgctacactgctactacatgagatctgctcttattggtcacgtactcacgttcacgctgaattaccgaatgtgttgtgtcttgtgtgtgtgtgttaaaggcattgtcctggtaaatggtggagacgagatggccgggtcgcgggagttatatttgtatatgttgccagctttctgagtttttcaagccagtgcggctcttcagtgcaggttccaagcg

SEQ ID NO: 16 - DNA, 2394 bp, Oryza sativa
  source 1..2394: mol_type = other DNA
ggaggaggaggagcggcggcggcggcggatcctatgtcgggggcaatcgcttcctcgccggcggctactctcttcctcgctggctcctcctcctcttcgccgcggcgcaggcggagcagggtgagcggggtttggtggcacttgtatggtggcacggggctgcggttgcattgggagcggcggggtttggttcgggacggagccgtcgtgtgctcggcgtcggccgccggtggtgaggatggcgtcgcgaaggcgaagacgaagtcagcggggagctcgaaggcggtcgctgtgcagggcagcacggccaaggctgatcatgttgaggattcagtttcatcacccaaatacgtgaaacctgccgttgcgaagcaaaatggagaggttgtgagcagagctaccaaatctgatgcaccagtgtctaaacccaaagttgacccgtcagttcctgcttccaaggctgaagctgatggtaatgcacaagctgtggaatcgaaggctgcactggacaagaaggaagacgtcggtgttgctgaaccgttggaagctaaggctgatgctggtggagatgcaggtgcagtaagctctgcagatgatagtgagaacaaggaatctggtccattggcagggccaaatgtgatgaatgtcattgtggtggcttctgaatgttctcctttctgtaaaacaggtggtcttggagatgttgtgggtgctttgcctaaggctctggcaagaagaggacatcgtgttatggttgtgataccaagatacggagaatatgcagaagccaaggatctaggtgtccgcaaacgttacagggtagctggacaggattcagaagtgagttattttcatgcttttatcgatggtgttgattttgtgtttctagaggcccctccctttcgacaccggcacaatgatatttatggaggagaaagatttgatgttttgaagcgtatgattttgttctgcaaagctgctgttgaggttccttggtttgctccatgtggtggttccatctacggtgatggcaatttagtcttcattgcaaatgattggcatactgcacttttacccgtatgtttgaaggcatattaccgagataatggcttgatgcagtatactcgctctgttcttgtgatacacaacattgctcatcagggtcgcggccctgtagatgacttcgccacgatggacttgcccgagcactacatcgatcatttcagactgtacgaccccgtcggcggtgagcacagcaacgtgttcgccgctggcctgaagatggctgaccgcgcggtcaccgtcagccatggctatctgtgggagatcaagacaatggacggtggctggggcctccacgagataataaaccacaacgactggaagctgcagggcatcgtgaatggcatcgacatggccgaatggaaccccgaggtggacgagcacctgcagtccgacggctacgccaactacacgttcgagacgctggacaccggcaagaaacagtgcaaggaggcgctgcagcggcagctgggcctgcaggtccgcgacgacgtgccgctgatcgggttcatcgggcggctcgaccaccagaagggcgtggacatcatcggcgacgcgatgccgtggatcgccgggcaggacgtgcaggtggtgatgctgggcacggggcggccggacctggaggagatgctgcggcggttcgagtcggagcacaacgacaaggtgcgcgggtgggtggggttctcggtgcagctggcgcaccggatcacggcgggcgccgacgtcctcctgatgccgtcgcggttcgagccgtgcgggctgaaccagctgtacgccatggcgtacagcaccgtgcccgtggtgcacgccgtgggcgggctccgcgacacggtggcgccgttcgacccgttcgccgacacggggctcgggtggacgttcgaccgcgccgaggcgaaccggatgatcgacgcgctcgggcactgcctcaacacgtaccggaactacaaggagagctggagggggctccaggcgcgcggcatggcgcaggacctcagctgggaccacgccgccgagctctacgaggacgtcctcgtcaaggccaagtaccagtggtgagctggtggtatttttcgcgacgcctctggatcccgccgtggatgggcgcgctttttctctagggcggctctgcgtcggagacgctcagacgcatcgcgcgcgttcgcgcccacacgatgactggcacgctgctgctgagacggcggcacgcgatgccatcatgccaccggacggtaggagcaacggtggaagtgaactgtactactggtgctacactgctactacatgagatctgctcttattggtcacgtactcacgttcacgctgaattaccga

SEQ ID NO: 17 - protein, 694 aa, Oryza sativa
MSGAIASSPAATLFLAGSSSSSPRRRRSRVSGVWWHLYGGTGLRLHWERRGLVRDGAVVCSASAAGGEDGVAKAKTKSAGSSKAVAVQGSTAKADHVEDSVSSPKYVKPAVAKQNGEVVSRATKSDAPVSKPKVDPSVPASKAEADGNAQAVESKAALDKKEDVGVAEPLEAKADAGGDAGAVSSADDSENKESGPLAGPNVMNVIVVASECSPFCKTGGLGDVVGALPKALARRGHRVMVVIPRYGEYAEAKDLGVRKRYRVAGQDSEVSYFHAFIDGVDFVFLEAPPFRHRHNDIYGGERFDVLKRMILFCKAAVEVPWFAPCGGSIYGDGNLVFIANDWHTALLPVCLKAYYRDNGLMQYTRSVLVIHNIAHQGRGPVDDFATMDLPEHYIDHFRLYDPVGGEHSNVFAAGLKMADRAVTVSHGYLWEIKTMDGGWGLHEIINHNDWKLQGIVNGIDMAEWNPEVDEHLQSDGYANYTFETLDTGKKQCKEALQRQLGLQVRDDVPLIGFIGRLDHQKGVDIIGDAMPWIAGQDVQVVMLGTGRPDLEEMLRRFESEHNDKVRGWVGFSVQLAHRITAGADVLLMPSRFEPCGLNQLYAMAYSTVPVVHAVGGLRDTVAPFDPFADTGLGWTFDRAEANRMIDALGHCLNTYRNYKESWRGLQARGMAQDLSWDHAAELYEDVLVKAKYQW

SEQ ID NO: 18 - DNA, 2025 bp, Triticum aestivum
  source 1..2025: mol_type = other DNA
atgtcgggggcaatagcttcctcgtccgcggcgttcctcttgctcgtggcctcctcctcctgctcctcctcgtcgccgcggcgctggcggagcagctcaccgcgctctttcgctgccgccggtacgcggctgcattgggaacggcgaggattctctcgggacggaccggtgccgtgccggagcccctgcgcagcagctggtggtgaggatggcgcggcggcaggggagagctcgaaggagggggtcgctgggctgcgcggcaagctcaaggctgctgaaaatgaggatccggtttcacaaaaatcagttgcatctgcacccaggctagacagtagcgttgcagagcaaaatggggcagctgcgaccagatccaaagccgatccgtcagctcctgtctcggcggcagcatcaggttattggaaagacgtcgttgtcgctgaacaggttggaactaaggttgacaccggtggagatgcagcagaagtgtcgagatctcccgttgacagtgagaacaaggggtctgctcctttggctggcccgaatgtgatgaatgtcatcgttgtggcttctgagtgtgctcctttctgtaaaacaggcgggcttggagatgtcgtgggtgctttgcccaaggctctggccagaagagggcatcgcgttatggttgtgataccaaagtatggcgattacgcagaagctcgtgatcttggtgttcgcaaacgttacaaggtagctggccaggattcagaagtgagttactttcactcttatatcgatggagttgattttgtattcttagaggccccgcccttccgccaccggcacaatgatatttatggaggagaaagaccggatgtgctgaagcgcatgattttgttctgcaaggctgctgtcgaggttcctaggtacgctctatgtggtggtactatctacggagatggcaatttggtcttcattgcaaacgattggcatactgcacttctacctgtttatctaaaggcatattaccgagacaatggcctgatgcagtatactcgttctgttctcgtgatccataacattgctcatcagggacgtggccctgtagatgacttcaacatcatggacttgcctgaccactacatgggtcacttcaaactgcatgacccccttggtggcgagcacaacaacgtgttcgccgctggcctgaagatggcagaccgggtggtcaccgtcagccatggctacatgtgggagctgaagacggtggaaggcggctggggcctccacgacataataaaccagaacgactggaaactggacggcatcgtgaacggcatcgacacggccgagtggaaccccgcggtcgacgtgcacctgcactccgacgactacaccaactacacccgagacacgctggacatcggcaagcggcagtgcaaggcggccctgcagcgcgagctgggcctgcaggtccgcgacgacgtgccgctcatcgggttcatcgggcggctggaccaccagaagggcgtggacatcatcgcggcggcgatgccgtggatcgcgcagcaggacgtgcagctggtgatgctgggcacggggcggccggacctggaggacatgctgcggcggttcgagggggagcaccgggacaaggtgcgcgggtgggtggggttctcggtgcggatggcgcaccggatcacggcgggcgcggacgtcctgctgatgccgtcgctgttcgagccgtgcgggctgaaccagctgtacgcggtggcgtacgggaccgtgcccgtggtgcacgccgtcggcgggctccgggacacggtggcgccgttcgacccgttcggcggcaccgggctggggtggacgttcgaccgcgcggacgccggcaggatgatcgacgcgctcgggcactgcctcaacacgtactggaactacaaggagagctggaggggcctccaggtccgcggcatgtcgcaggacctcagctgggaccacgccgccgagctctacgagaacgtcctcgtcaaggccaagtaccagtggtga

SEQ ID NO: 19 - protein, 674 aa, Triticum aestivum
MSGAIASSSAAFLLLVASSSCSSSSPRRWRSSSPRSFAAAGTRLHWERRGFSRDGPVPCRSPCAAAGGEDGAAAGESSKEGVAGLRGKLKAAENEDPVSQKSVASAPRLDSSVAEQNGAAATRSKADPSAPVSAAASGYWKDVVVAEQVGTKVDTGGDAAEVSRSPVDSENKGSAPLAGPNVMNVIVVASECAPFCKTGGLGDVVGALPKALARRGHRVMVVIPKYGDYAEARDLGVRKRYKVAGQDSEVSYFHSYIDGVDFVFLEAPPFRHRHNDIYGGERPDVLKRMILFCKAAVEVPRYALCGGTIYGDGNLVFIANDWHTALLPVYLKAYYRDNGLMQYTRSVLVIHNIAHQGRGPVDDFNIMDLPDHYMGHFKLHDPLGGEHNNVFAAGLKMADRVVTVSHGYMWELKTVEGGWGLHDIINQNDWKLDGIVNGIDTAEWNPAVDVHLHSDDYTNYTRDTLDIGKRQCKAALQRELGLQVRDDVPLIGFIGRLDHQKGVDIIAAAMPWIAQQDVQLVMLGTGRPDLEDMLRRFEGEHRDKVRGWVGFSVRMAHRITAGADVLLMPSLFEPCGLNQLYAVAYGTVPVVHAVGGLRDTVAPFDPFGGTGLGWTFDRADAGRMIDALGHCLNTYWNYKESWRGLQVRGMSQDLSWDHAAELYENVLVKAKYQW

SEQ ID NO: 20 - DNA, 2302 bp, Sorghum bicolor
  misc_feature 2247: note = n is a, c, g, or t
  source 1..2302: mol_type = other DNA
cggaagcagcagcagcagtagcgtgaggcatccccatgtcggcggcaatctcctcctcgtcctcggcttttctcctcctcatcgcgtcctcctcgccgcggcgcaggcggggcagggtgggcgctgctctgcgctcctacggctacagcggcgcggagctgcggttgcattgggcgcggcgggccccgtcacgggatgcggcggcggtagtagtgcgcgccgcagcggcgccggccgggggtgaggagggcgaggaggccgcggcggcagggatgagctcctcgtccaagggcgtcgccgtgcagggcagcaaggccaaggctgtagattctacttcacctcccaaacctgtgacttctgcgacgaagcaaagtccgagcgctgcaaatcaaaacggaacggttgggagcagcagtgcaagcaaatctgccacgccggtgtccgaactcaaagctgaaacatcagctcctgtcaccaagactgaaaccgatgccagtgcgaaggttgaagagccgaagcccgcggtggatgatgctaaaccggtggaaagcataggcgttgctgagccggtggatgctaaggtggacgtcgctgcagctactgatgtggaggcgagtgctgctgatgacagtgaggacaagaaacctggccctttggctgggcctaatgtgatgaatgtcgtcgtggtggcttctgaatgtgctcctttctgcaagacaggtggccttggagatgttgtgggtgctttgccgaaggctttggcgaggagagggcaccgcgttatggtcgtgataccaagatatggagaatatgcagaagcccgagatttaggtgtgcggaaacgttacagggtagctggacaggattcagaagttacttactttcactcttacattgatggagttgattttgtattcatagaagccccacccttccggcaccggcacaataatatttatggaggagaaagattggatattttgaagcgcatgattttgttctgcaaggccgctgttgaggttccatggtatgctccatgtggcggtaccgtctatggtgatggcaacttagttttcattgctaatgattggcatactgcacttctgccggtctatctaaaggcctattaccgggacagtggtttgatgcagtatgctcgctctgtgcttgtgatacacaacattgctcatcagggtcgtggccctgtagacgacttcgtcaattttgacttgcctgaacattacatcgatcacttcaaactgtatgacaatatcggtggcgatcacagcaacgttttcgccgcgggcctgaaggcggcagaccgggtggtgaccgtcagcaatggctacctgtgggagctgaagacttcggaaggtgggtggggcctccacggcatcataaaccagaacgactggaagctgcagggcatcgtgaacggcatcgacatgagcgagtggaaccccgctgtggacgtgcacctccactccgacggctacaccaactacacgttcgagacgctggacactggcaagcggcagtgcaaggccgccctgcagcggcagctcggcctgcaggtccgcgacgacgtgccactgatcgggttcatcggtcgcctggatggacagaagggcgtggacctcatcggcgacgcgatccactggatcgcggggcaggacgtgcagctggtgatgctgggcaccgggcgccccgacctggaggacatgctgcggcggtgcgaggcggagcacaacgacaaggtgcgcgcgtgggtcggtttctcggttcccctggcgcaccgcatcacggcgggcgccgacgtcctgctgatgccgtcgcggttcgagccgtgcgggctgaaccagctctacgcgatggcgtacgggaccgtgcccgtggtgcacgccgtgggcgggctccgggacacggtggcgccgttcgacccgttcaacgacaccgggctcgggtggacgttcgaccgcgccgaggcgaatcggatgatcgacgcgctccatcactgcctcaacacgtaccggaacttcaaggagagctggcgcggcctgcaggagcgcggcatggcgcaggacctgagctgggaccacgccgccgtgctgtatgaggacgtgctcgtcaaggccaagtaccagtggtgactggtgagtgggtttaactggcgacgccgacgccctcctgtcgcgggacctggacgcgttgggcatgttatttcggcggctcttctcccctcggctntgatgcgtgcggcgcaattgcgccgcccgggcggagggtgatggtggttggccta

SEQ ID NO: 21 - protein, 704 aa, Sorghum bicolor
MSAAISSSSSAFLLLIASSSPRRRRGRVGAALRSYGYSGAELRLHWARRAPSRDAAAVVVRAAAAPAGGEEGEEAAAAGMSSSSKGVAVQGSKAKAVDSTSPPKPVTSATKQSPSAANQNGTVGSSSASKSATPVSELKAETSAPVTKTETDASAKVEEPKPAVDDAKPVESIGVAEPVDAKVDVAAATDVEASAADDSEDKKPGPLAGPNVMNVVVVASECAPFCKTGGLGDVVGALPKALARRGHRVMVVIPRYGEYAEARDLGVRKRYRVAGQDSEVTYFHSYIDGVDFVFIEAPPFRHRHNNIYGGERLDILKRMILFCKAAVEVPWYAPCGGTVYGDGNLVFIANDWHTALLPVYLKAYYRDSGLMQYARSVLVIHNIAHQGRGPVDDFVNFDLPEHYIDHFKLYDNIGGDHSNVFAAGLKAADRVVTVSNGYLWELKTSEGGWGLHGIINQNDWKLQGIVNGIDMSEWNPAVDVHLHSDGYTNYTFETLDTGKRQCKAALQRQLGLQVRDDVPLIGFIGRLDGQKGVDLIGDAIHWIAGQDVQLVMLGTGRPDLEDMLRRCEAEHNDKVRAWVGFSVPLAHRITAGADVLLMPSRFEPCGLNQLYAMAYGTVPVVHAVGGLRDTVAPFDPFNDTGLGWTFDRAEANRMIDALHHCLNTYRNFKESWRGLQERGMAQDLSWDHAAVLYEDVLVKAKYQW

SEQ ID NO: 22 - DNA, 2569 bp, Zea mays
  source 1..2569: mol_type = other DNA
acgctctaggctctcgtttcctcgtactggtacaagcgggacgggagcagcagcagtagcgtgatccatccccatggcgggggcaacctcttcctcgtccgtgtctttgatcctcatcctcgtcgcgtcctcctcgccgcggcgcagccggggcagggtgggcattgctctgcgctcgtacggctacagcggcgccgagctgcggttgcattgggcgcgtcggggctcgtctcgggatggaccggcggtagtgcgcgccgcagcggttccggccgggggtgagggcgaggacgccgcggcggtagggaagagctcctcgtccaaggcggtcgccgtgcagggcagcgagtccaaggctgtatattctacttcacctcccaaaactgcgacgtctgcgccgaagcaaaaccagagcgctgcaaagcaaaacggaacagttgggagcagcagtgcgagcaaatccccggcgcctgcggtatctgaatccaaagctgaaccatctgctcctgtcaccaagacagaaaccgatgccagtgcgaaggttgaagagctgaagccctcagtggatgatgctaaacccgtagaaagcataggcatccctgagccggtggatgctaaggtggacgccgctgcagctactgatgccgcggcgagtgctgctgatgacactgaagacaaggaacatggccctttggctgggcctaatgtgatgaatgtcgtcgtggtggcttctgaatgcgctcctttctgcaagacaggtggccttggagatgttgtgggtgctttgcccaaggctttggcgaggagaggacaccgtgttatggtcgtgataccaagatatggagagtatgcagaagcccgggatttaggtgtaaggagacgttacagggtggctggacaggattcagaagttacttattttcactcttacattgatggcgttgattttgtattcatagaagcccctcccttccggcaccggcacaatgatatttatggaggagaaagatcggatgttttgaagcgcataattttgttctgcaaggccgctgttgaggttccatggtacgctccatgtggtggtaccgtctatggtgatggcaatttagttttcattgctaatgattggcatacggcacttcttccggtctacctaaaagcctactaccgggacaatggtttgatgcagtatgctcgctcagtgcttgtgatacacaacattgctcatcagggtcgtggccctgtagacgacttcgtcaactttgacttgcctgaacattacatcgatcacttcaaactgtacgacaatatcggtggcgagcactgcaacgtttttgccgcagggctgaagatggcagaccaggtggtgaccgtcagtaatggatacctatgggagctgaggacatcggaaggtgggtggggcctccacgacatcataaaccagaacgactggaagctgcagggcatcgtgaacggcatcgacatgagcgagtggaaccccgctgtggacgtgcacctccactccgacgaatacaccaactacacgttcgagacgctggacacgggcaagcggcagtgcaaggctgccctgcagcggcagctcggcctgcaggtccgcgacgacgtgccactaatcgggttcatcgggcggctggaccaccagaagggcgtggacatcatcgccgacgcgatccactggatcgcgggacaggacgtgcagcttgtgatgctgggcacggggcgccccgacctggaggacatgctgcggcggtgcgaggcggagcacagcgacaaggtgcgcgcgtgggtggggttctcggtgcccttggcgcaccgcatcacggcgggcgcggacgtcctgctgatgccgtcgcggttcgagccgtgcgggctgaaccagctctacgcgatggcgtacgggaccgtgcccgtggtgcacgccgtgggcgggctccgggacacggtggcgccgttcgacccgttcaacgacaccgggctggggtggacgttcgaccgcgcggaggcgaatcggatgatcgacgcgctctcgcactgcctcaacacgttccggaactacaaggagagctggcgcggcctggaggcgcgcggcatggcgcaggacctcagctgggaccacgccgccgtgctgtatgaggacgtgctcgtcaaggccaagtaccagtggtaagtgagttacgccgacgcccttcatgtcgcgggacagtcctggacgccctgggcactgcgtgctcttctcacgcggctttgctgcgtgcggcgcatttgctcaattggcgcggggcgatggtggttggcctacgtcggcttcggctaggtgccttcggattttcagcttcttaccaccaaaacaccaaaagctgctgcggactattaaacgctcagtttttcagttagcttctataaaaatcgatcgtaaaaatcatctataatcaacataaacacataatggacggagtacttttaataatagaaatctatcgctttgtagatcttaccctatggacaccttcatcttgaaaattgtttggttgaaataacagtgagttgt

SEQ ID NO: 23 - protein, 704 aa, Zea mays
MAGATSSSSVSLILILVASSSPRRSRGRVGIALRSYGYSGAELRLHWARRGSSRDGPAVVRAAAVPAGGEGEDAAAVGKSSSSKAVAVQGSESKAVYSTSPPKTATSAPKQNQSAAKQNGTVGSSSASKSPAPAVSESKAEPSAPVTKTETDASAKVEELKPSVDDAKPVESIGIPEPVDAKVDAAAATDAAASAADDTEDKEHGPLAGPNVMNVVVVASECAPFCKTGGLGDVVGALPKALARRGHRVMVVIPRYGEYAEARDLGVRRRYRVAGQDSEVTYFHSYIDGVDFVFIEAPPFRHRHNDIYGGERSDVLKRIILFCKAAVEVPWYAPCGGTVYGDGNLVFIANDWHTALLPVYLKAYYRDNGLMQYARSVLVIHNIAHQGRGPVDDFVNFDLPEHYIDHFKLYDNIGGEHCNVFAAGLKMADQVVTVSNGYLWELRTSEGGWGLHDIINQNDWKLQGIVNGIDMSEWNPAVDVHLHSDEYTNYTFETLDTGKRQCKAALQRQLGLQVRDDVPLIGFIGRLDHQKGVDIIADAIHWIAGQDVQLVMLGTGRPDLEDMLRRCEAEHSDKVRAWVGFSVPLAHRITAGADVLLMPSRFEPCGLNQLYAMAYGTVPVVHAVGGLRDTVAPFDPFNDTGLGWTFDRAEANRMIDALSHCLNTFRNYKESWRGLEARGMAQDLSWDHAAVLYEDVLVKAKYQW

(19) United States
(12) Patent Application Publication
(10) Pub. No.: US 2025/0163444 A1
(43) Pub. Date: May 22, 2025

(54) HIGH FRUCTAN CEREAL PLANTS

(71) Applicant: Commonwealth Scientific and Industrial Research Organisation, Acton (AU)

(72) Inventors: Colin Leslie Dow Jenkins, Evatt (AU); Bryan Charles Clarke, Pearce (AU); Zhongyi Li, Kaleen (AU); Matthew Kennedy Morell, Aranda (AU)

(73) Assignee: Commonwealth Scientific and Industrial Research Organisation, Acton (AU)

(21) Appl. No.: 19/029,998

(22) Filed: Jan. 17, 2025

Related U.S. Application Data
(63) Continuation of application No. 17/462,700, filed on Aug. 31, 2021, now Pat. No. 12,241,073.
Application No. 17/462,700 is a division of application No. 15/671,970, filed on Aug. 8, 2017, now Pat. No. 11,111,498.
Application No. 15/671,970 is a division of application No. 13/008,746, filed on Jan. 18, 2011, now Pat. No. 9,752,157.
Application No. 13/008,746 is a continuation-in-part of application No. PCT/AU2009/000911, filed on Jul. 16, 2009.
(60) Provisional application No. 61/135,111, filed on Jul. 17, 2008.
(60) Provisional application No. 61/135,361, filed on Jul. 18, 2008.

(30) Foreign Application Priority Data
Feb. 13, 2009 (AU) 2009200577

(51) Int. Cl. C12N 15/82; G01N 33/50

(52) U.S. Cl. CPC C12N 15/8245; G01N 33/50; G01N 2400/38

(57) ABSTRACT

The invention provides cereal plants having a high level of fructan useful for the production of a range of food, beverage, nutraceutical and pharmaceutical products. The invention provides methods of producing high-fructan products from plants modified to comprise a reduced level of an endogenous polypeptide with starch synthase activity, and products so produced. In some embodiments, plants are modified by introduction of an agent such as a nucleic acid molecule which down regulates endogenous starch synthase II gene expression.

### FIELD

This application is a continuation of U.S. application Ser. No. 17/462,700, filed Aug. 31, 2021, now allowed, which is a divisional of U.S. application Ser. No. 15/671,970, filed Aug. 8, 2017, now U.S. Pat. No. 11,111,498, filed Sep. 7, 2021, which is a divisional of U.S. application Ser. No. 13/008,746, filed Jan. 18, 2011, now U.S. Pat. No. 9,752,157, issued Sep. 5, 2017, which is a continuation-in-part of PCT International Application No. PCT/AU2009/00911, filed Jul. 16, 2009, which claims the benefit of U.S. Provisional Applications Nos. 61/135,111, filed Jul. 17, 2008, and 61/135,361, filed Jul. 18, 2008, and claims priority of Australian Patent Application No. 2009200577, filed Feb. 13, 2009, the contents of each of which are hereby incorporated by reference in their entirety into this application.

This application incorporates by reference nucleotide and/or amino acid sequences which are present in the file named “250117_79593-BZZA_SequenceListing_AD.xml,” which is 63 kilobytes in size, and which was created Jan. 17, 2025 in the IBM-PC machine format, having an operating system compatibility with MS-Windows, which is contained in the xml file filed Jan. 17, 2025 as part of this application.

The present specification describes cereal plants having a high level of fructans useful for the production of a range of food, beverage, nutraceutical and pharmaceutical products.

### BACKGROUND

Fructans are polymers of fructose which are synthesized from sucrose and used as storage or reserve carbohydrates by many plants. They consist of fructosyl residues polymerized to sucrose, and therefore comprise fructosyl units in addition to one glucose unit. In view of this composition, they are highly soluble in water. The linkages between the fructosyl-residues are either exclusively of the β(1-2) type forming a linear molecule (inulin) in which the fructosyl residues are attached to the fructosyl residue of the sucrose starter, or of the β(2-6) type (levan), or both linkage types occur in branched fructans (graminans). Inulins are present in plants belonging to the Asterales (e.g. chicory) or the Liliaceae (e.g. onion). All fructans found in the dicotyledons, as well as some monocotyledons are of this type. The inulin in onion is termed neo-series inulin and has two linear β(1-2)-linked fructosyl chains, one attached to the C1 of the fructosyl residue of the sucrose and one attached to the C6 of the glucosyl residue of the sucrose. Levans are typically found in monocotyledons such as the Poaceae (e.g. grasses) and in almost all bacterial fructans. Graminans which consist of β(2-6)-linked fructose units with β(1-2) branches and are therefore more complex structures can also be present in cereals, and can be mixed with levans.

The degree of polymerization (DP) and distribution of linkage types are characteristic of different plant species. Since a range of DP are often seen in any one species, fructans typically show a disperse molecular weight. In contrast to the high molecular weight of fructan (levan, 1-5×10⁶ Da) elaborated as an extracellular polysaccharide by some bacteria, plant fructans are much smaller by 2-3 orders of magnitude.

Fructans, rather than starch, occur naturally as the primary reserve carbohydrate in about 10-15% of higher plants including chicory, artichoke, asparagus, dahlia and the onion family, primarily in the perennating organs. Fructans are mostly stored in taproots (e.g. chicory) or tubers (e.g. dahlia, Jerusalem artichoke) or bulbs (e.g. onion). In grasses and cereals, fructans are mainly stored in the stems and leaf bases and used as a reserve carbohydrate for growth and seed production. Fructan also occurs as a temporary storage form in the vegetative tissues of forage grasses and cereals, but only at low levels in cereal grain. Despite this, wheat products are the primary source of fructan in the Western diet. Onions are the second largest source of naturally occurring fructans in the American diet, accounting for about 25% of total consumption (Moshfegh et al., J Nutr. 129(Suppl): 1407S-11S, 1999).

Cereals such as wheat and barley accumulate, mainly in vegetative tissues, branched graminan-type fructans containing both β-(2,1) and β-(2,6) fructosyl linkages. These mostly have a low DP, such as 1-6-kestotetraose (bifurcose) which is the major fructan oligosaccharide accumulating in crown tissues and leaves of cereals exposed to chilling. Fructans are naturally present in various cereal grains (White and Secor, Arch Biochem Biophys. 44: 244-5, 1953; Henry and Saini, Cereal Chem. 66: 362-365, 1989; Schnyder, New Phytol 123: 233-245, 1993). Wheat grain has been reported to contain 0.6-2.6% (w/w) fructan.

Fructan is synthesized directly from sucrose as the sole precursor, without any known involvement of phosphorylated sugars or nucleotide co-factors, by the activity of specific fructosyltransferases (FTs). Synthesis generally occurs in vacuoles, outside of the plastid, and accumulation of fructan occurs in vacuoles of both photosynthetic and storage cells. Fructan synthesis in plants is initiated by a sucrose:sucrose 1-fructosyltransferase (1-SST, EC 2.4.1.99) using sucrose both as fructosyl donor and acceptor to produce 1-kestose, the shortest β(1-2) linked fructan) and glucose. 1-SST is found in all fructan-producing plants. Longer chain inulins are formed by the action of a second enzyme, fructan:fructan 6-fructosyltransferase (1-FFT, EC 2.4.1.100) which adds fructosyl residues by β(1-2) linkages. 1-FFT can use 1-kestose or fructans as fructose donors and therefore can transfer fructosyl residues from one fructan chain to another. Synthesis of the neo-series fructans requires fructan:fructan 6G-fructosyltransferases (6G-FFT). In the case of cereals such as wheat and barley, the next step of fructan synthesis is mediated by a sucrose:fructan 6-fructosyltransferase (6-SFT, EC 2.4.1.10) which transfers a fructosyl unit from a further sucrose molecule to fructan with a β(2-6) linkages, to extend the fructan polymer. Fructosyl transfer to 1-kestose, the smallest branched fructan, forms the tetrasaccharide bifurcose. It remains to be shown whether or not additional FTs are involved in fructan synthesis of grasses or cereals, but the combined action of 1-SST, 1-FFT, 6-FFT and 6G-FFT may be involved in graminan synthesis.

Many plant fructosyltransferases have been sequenced during the last few years, and the data clearly indicate a high homology to the vacuolar, acid invertases (β-fructosidases). These enzymes are all members of the glycoside hydrolase family 32 (GH32) and share three highly conserved regions characterized by the motifs (N/S)DPNG (also called β-fructosidase motif), RDP, and EC. The aspartate of the (N/S)DPNG motif provides a nucleophile in the catalysis, the glutamate of the EC-motif as a proton donor, and the aspartate of the RDP motif as transition state stabilizer in the transfructosylation reaction.

Fructans are catabolised by fructan exohydrolases (FEH; EC 3.2.1.80) which are specialized for fructans, and invertases such as acid invertase (EC 3.2.1.26) which hydrolyse sucrose. Genes encoding fructan exohydrolase have been isolated from wheat (Van den Ende et al., Plant Physiol. 131(2): 621-631, 2003). Other enzymes such as sucrose phosphate synthase (SPS; EC 2.4.1.14) and sucrose synthase (EC 2.4.1.13) are associated with fructan remobilization from the stems.

Fructans are non-starch carbohydrates with potentially beneficial effects as a food ingredient on human health (Tungland and Meyer, Comprehensive Reviews in Food Science and Food Safety, 2: 73-77, 2002; Ritsema and Smeekens, Curr. Opin. Plant Biol. 6: 223-230, 2003). The human digestive enzymes α-glucosidase, maltase, isomaltase and sucrase are not able to hydrolyse fructans because of the β-configuration of the fructan linkages. Furthermore, humans and other mammals lack the fructan exohydrolase enzymes that break down fructans and therefore dietary fructans avoid digestion in the small intestine and reach the large intestine intact. However, bacteria there are able to ferment fructans and thereby utilize them as, for example, an energy or carbon source for growth and production of short-chain fatty acids (SCFA). Dietary fructans therefore are able to stimulate the growth of beneficial bacteria such as bifidobacteria in the colon, which aids in prevention of bowel disorders such as constipation and infection by pathogenic gut bacteria. Dietary fructan also enhances nutrient absorption from diets, particularly calcium and iron, possibly via production of SCFA which in turn reduce luminal pH and modify calcium speciation and hence solubility, or exert a direct effect on the mucosal transport pathway, thereby improving the mineralization of bone and reducing the risk of iron deficiency anaemia. In addition, a high-fructan diet can improve the health of patients with diabetes and reduce the risk of colonic cancers by suppressing aberrant crypt foci which are precursors of colon cancer (Kaur and Gupta, J. Biosci. 27: 703-714, 2002).

Attempts have been made to enhance fructan production in transgenic plants by introduction and expression of genes encoding 1-SST and 1-FFT. Generally, fructan accumulation levels were less than 2% (w/w) for plants transformed with bacterial genes and less than 1% (w/w) using plant genes. In some exceptions, concentrations of 6-16% on a fresh weight basis were achieved and compare favourably with naturally occurring maximal starch and fructan content in leaves and tubers (Sevenier et al., Nature Biotechnol. 16: 843-846, 1998; Hellwege et al., Proc. Natl. Acad. Sci. U.S.A. 97: 8699-8704, 2000). Transformants expressing bacterial fructan synthesis genes sometimes exhibited aberrant phenotypes such as stunting, leaf bleaching, necrosis, reduced tuber number and mass, tuber cortex discoloration, reduction in starch accumulation, and chloroplast agglutination.

There is therefore a need for efficient production of fructan from plant sources at low cost.

### SUMMARY

Throughout this specification, unless the context requires otherwise, the word “comprise”, or variations such as “comprises” or “comprising”, will be understood to imply the inclusion of a stated element or integer or group of elements or integers but not the exclusion of any other element or integer or group of elements or integers.

As used herein the singular forms “a” “an” and “the” include plural aspects unless the context clearly dictates otherwise. Thus, for example, reference to “a mutation” includes a single mutation, as well as two or more mutations; reference to “an agent” includes one agent, as well as two or more agents; and so forth.

Nucleotide and amino acid sequences are referred to by a sequence identifier number (SEQ ID NO:). The SEQ ID NOs: correspond numerically to the sequence identifiers <400>1 (SEQ ID NO:1), <400>2 (SEQ ID NO:2), etc. A summary of sequence identifiers is provided in Table 7. A sequence listing is provided after the claims.

Genes and other genetic material (e.g. mRNA, nucleic acid constructs etc) are represented herein in italics while their proteinaceous expression products are represented in non-italicised form. Thus, for example starch synthase II (SSII) polypeptide is the expression product of SSII nucleic acid sequences.

Representative examples of the nucleic acid and amino acid sequences of SSII molecules are provided in the sequence listing further described in Table 7. The terms SSII or SSII encompass all functional homologs in any plant species including cereal plants and including SSII molecules such as SSIIa, SSIIb, SSIIa-2, SSIIa-B, SSIIa-D and SSII-2 etc. In a particular embodiment, the SSII is SSIIa.

Bibliographic details of the publications referred to by author in this specification are collected at the end of the description.

The reference in this specification to any prior publication (or information derived from it), or to any matter which is known, is not, and should not be taken as an acknowledgment or admission or any form of suggestion that that prior publication (or information derived from it) or known matter forms part of the common general knowledge in the field of endeavour to which this specification relates.

Each embodiments described herein is to be applied mutatis mutandis to each any every embodiment unless specifically stated otherwise.

Accordingly in one embodiment, the present specification describes a method of producing a high-fructan product, wherein the method comprises: (i) obtaining or producing a cereal plant or grain or flour therefrom wherein the cereal grain or flour comprises at least 3%, preferably at least 4%, fructan as a percentage of the cereal grain or flour weight; and (ii) processing the plant, grain or flour to produce the product.

In particular embodiments, the grain is characterized by a combination of two parameters: the percent fructan in the grain by weight, and the starch content of the grain by weight. For the first parameter, the percentage fructan of the cereal grain by weight is at least 3%, preferably at least 4%, at least 5%, at least 6%, at least 7%, at least 8%, at least 9% or at least 10% as shown herein For the second parameter, the starch content of the grain by weight, is at least 30%, preferably at least 35%, at least 36%, at least 37%, at least 38%, at least 39% or at least 40% as a percentage of the total grain weight. The invention includes each and every specific combination of these two parameters with respect to the grain, products obtained therefrom, methods of obtaining and using the grain, and uses of the grain and products therefrom. Reference herein to “high fructan” is used merely to indicate that the product is produced from the herein disclosed modified grain having an elevated level of fructan compared to an unmodified or control form of the grain.

In another embodiment, the method comprises: (i) obtaining or producing a cereal plant or grain or flour therefrom wherein wherein the cereal plant or grain is modified to comprise a reduced level of an endogenous polypeptide with starch synthase II (SSII) activity relative to an unmodified control; and (ii) processing the plant, grain or flour to produce the product. Various forms of this aspect of the invention are described in the Examples. Methods for reducing the level of an endogenous polypeptide with starch synthase II (SSII) activity in cereal plants relative to an unmodified or control plant are known in the art as described herein and in the common general knowledge. In particular embodiments, the grain is characterized by the same combination of two parameters as described in paragraph 0021.

In some embodiments, the methods further comprise: (iii) assessing the level or type of fructan in the cereal plant or grain or flour therefrom, or the product therefrom.

In several embodiments, the cereal grain is wholegrain which may be cracked, ground, polished, milled, kibbled, rolled or pearled grain. In some embodiments, the present invention extends to monocotyledonous cereal plants selected from the group consisting of barley, wheat, rice, maize, rye, oat and sorghum. In further embodiments, the plant is tetraploid wheat, maize, rye, rice, oat or sorghum, or hexaploid wheat or barley.

In a particular embodiment, the plant is a barley shrunken grain mutant designated M292 or M342 described in International Publication No. WO 02/37955.

As illustrated in the Examples, the fructan of the present invention in some embodiments comprises a degree of polymerization from about 3 to about 12.

In one application of the present invention, the product is a food or beverage product or a pharmaceutical composition. In a particular embodiment, the product is isolated fructan. non-limiting examples of food or beverage products include, grain, flour, breakfast cereal, biscuit, muffin, muesli bar, noodle, corn, a sweetening agent, a low calorie additive, a bulking agent, a dietary fibre, a texturizing agent, a preservative, a probiotic agent or the like or any combination of these.

In some further embodiments, the cereal plant or grain of the present invention is modified to comprise a reduced level of an endogenous polypeptide with starch synthase II (SSII) activity relative to an unmodified control. As known to those of skill in the art a wide range of methods are available for reducing the level of an endogenous polypeptide in a plant. In some embodiments, the plant comprises a mutation in an endogenous gene encoding a polypeptide with SSII activity wherein the mutation reduces the expression of the gene encoding SSII in the plant or leads to the expression of SSII with reduced level or activity. In other embodiments, the level of SSII activity is reduced by introducing into said plant a nucleic acid molecule which down-regulates the expression of a gene encoding SSII in the plant. In some embodiments, the nucleic acid molecule comprises a gene-silencing chimeric gene, an antisense, ribozyme, co-expression dsRNA molecule, or other exogenous nucleic acid molecule that down-regulates endogenous SSII expression. In preferred embodiments, the grain is characterized by the same combination of two parameters as described in paragraph 0021.

In another aspect the present invention provides a method of producing a cereal plant or grain therefrom which has increased levels of fructan compared to a control plant, wherein the method comprises: (i) introducing into said plant an agent which down-regulates the level or activity of endogenous starch synthase II (SSII) in the plant relative to a control plant, or a mutation in an endogenous gene encoding SSII in the plant. As described further herein in some embodiments, the agent comprises a nucleic acid molecule which down-regulates endogenous SSII gene expression. Illustrative nucleic acid molecules include a gene-silencing chimeric gene, an antisense, ribozyme, co-expression dsRNA molecule, or other exogenous nucleic acid molecule that down-regulates endogenous SSII expression.

In a further embodiment of this aspect of the invention the method comprises assessing the level, activity or type of fructan in the plant or grain therefrom. In some embodiments, the increased level of fructan is at least twice, preferably at least 3×, at least 4×, at least 5×, at least 6×, at least 7×, at least 8×, at least 9× or at least 10×, that of a control plant or the plant prior to the introduction of the agent or mutation.

In another aspect, the present specification provides an isolated or genetically modified cereal plant or grain or flour therefrom wherein the grain or flour comprises at least 3%, preferably at least 4% fructan as a percentage of the cereal grain or flour weight. Preferably, the plant or grain or flour is used, or is for use, in the production of a product to increase the level of fructan or non-starch carbohydrate in said product. In some embodiments, the percentage fructan of the cereal grain or flour by weight is at least 5%, at least 6%, at least 7%, at least 8%, at least 9% or at least 10% as shown herein. In preferred embodiments, the grain is characterized by the same combination of two parameters as described in paragraph 0021. As is readily apparent, the invention includes the flour, fructan and food products produced from each of these preferred embodiments of grain.

Accordingly, the present invention contemplates, cereal grain, flour or fructan produced from the plant or grain as described herein. In some embodiments, the cereal grain or flour comprises a starch content of at least 30%, preferably at least 35%, at least 36%, at least 37%, at least 38%, at least 39% or at least 40% as a percentage of the total grain weight.

In some embodiments, the cereal plant is not barley or hexaploid wheat.

In particular embodiments, the cereal grain or flour as described herein comprising a reduced level or activity of a polypeptide having SSII activity.

In some embodiments, the present invention provides fructan, grain or flour produced from the plant or grain or flour as described herein. The inventors contemplate, for example, the use of fructan isolated from the subject plant, grain or flour in a food as a sweetening agent, a low calorie additive, a bulking agent, a dietary fibre, a texturizing agent, a preservative, a probiotic agent or the like or any combination of these. In some embodiments, the inventors contemplate the use of a grain or flour or frunctan isolated from a plant, grain or flour as described herein in the production of a food product to increase the level of frunctan in said food product. In preferred embodiments, the grain is characterized by the same combination of two parameters as described in paragraph 0021.

In some embodiments, the food product comprises a food ingredient at a level of at least 10% on a dry weight basis, wherein the food ingredient is a cereal grain comprising at least 3%, preferably at least 4%, fructan on a weight basis or wholemeal or flour obtained therefrom wherein the wholemeal or flour comprises at least 3%, preferably at least 4% fructan on a weight basis. In preferred embodiments, the grain is characterized by the same combination of two parameters as described in paragraph 0021.

In yet another aspect, the present invention provides a method of identifying a variety of cereal grain which has increased levels of fructan comprising: (i) obtaining cereal grain which is altered in starch via synthesis or catabolism; (ii) determining the amount of fructan in the grain, (iii) comparing the level of fructan to that in wild-type grain which is not altered in starch via synthesis or catabolism, and (iv) if the fructan level is increased in the altered grain, selecting the grain. In some embodiments, the method further comprises mutagenesis or plant cell transformation prior to step (i).

In another embodiments, a method is provided for determining the amount of fructan in cereal grain, comprising the steps of (i) obtaining grain comprising at least 3%, preferably at least 4% fructan on a weight basis, processing the grain so as to extract the fructan, and measuring the amount of extracted fructan so as to determine the amount of fructan in the grain.

In a further embodiment, the present invention contemplates a method for preparing a food or beverage, comprising mixing a high-fructan product obtained by the herein disclosed methods with another food or beverage ingredient.

In another embodiment, the present invention provides a method for providing fructan to improve one or more indicators of health in a subject in need thereof, wherein the method comprises administering, to the subject, a composition comprising cereal grain or flour therefrom comprising at least 3%, preferably at least 4%, fructan on a weight basis or fructan obtained therefrom. In some embodiments, the grain, flour or fructan is in the form of a food product, a beverage or a pharmaceutical composition. In other embodiments, the one or more indicators of health is an increased number of beneficial intestinal bacteria, reduced number of aberrant crypt foci, increased mineral absorption, reduced level of insulin, reduced glycaemic index, reduced glycaemic load, reduced blood glucose, reduced blood pressure, reduced body weight, reduced blood cholesterol level, increased HDL cholesterol level, increased bone density, increased calcium levels, more frequent bowel movement, or improved blood serum cardiovascular profile. In preferred embodiments, the grain is characterized by the same combination of two parameters as described in paragraph 0021.

The present invention provides a method for ameliorating one or more symptoms of a condition associated with low levels of dietary fructan in a subject in need thereof, said method comprising administering orally to the subject grain comprising at least 3%, preferably at least 4%, fructan as a percentage of the cereal grain weight or a processed product comprising the fructan obtained therefrom for a time and under conditions sufficient to ameliorate one or more symptoms. The condition, in some embodiments, is selected from the group consisting of diabetes, obesity, heart disease, hypertension, constipation, osteoporesis and cancer. The method may comprise the step of determining that the subject may benefit from increased intake of dietary fructan. In preferred embodiments, the grain is characterized by the same combination of two parameters as described in paragraph 0021.

Any subject who could benefit from the present methods or compositions is encompassed. The term “subject” includes, without limitation, humans and non-human primates, livestock animals, companion animals, laboratory test animals, captive wild animals, reptiles and amphibians, fish, birds and any other organism. A subject, regardless of whether it is a human or non-human organism may be referred to as a patient, individual, subject, animal, host or recipient. In a particular embodiment the subject is a human.

The above summary is not and should not be seen in any way as an exhaustive recitation of all embodiments of the present invention.

### BRIEF DESCRIPTION OF THE DRAWINGS

FIG. 1 is a graphical representation showing the soluble carbohydrate profile of mutant ‘M292’ and wild type ‘Himalaya’ mature barley grains. HPAE chromatographic elution profile of grain extracts indicating increased levels of hexose, sucrose, maltose and fructo-oligosaccharides in the mutant M292 compared to wild type Himalaya grain. Glc, glucose; Fru, fructose; Suc, sucrose; Mal, maltose; 1-K, 1-kestose; numerals indicate tentative degree of polymerization (DP) of fructo-oligosaccharides.

FIG. 2 is a graphical representation of data showing the fermentation properties of modified cereal products. Extract of barley grain mutant 292 and standards (inulin, lactulose, glucose) were fermented in carbon-limited fermentation media. Short chain fatty acid (SCFA) production was measured. This indicated that the lower doses of mutant 292 produced comparable fermentation to that of inulin (see total SCFA 1″ & 2^{nd} column (1% 292 and inulin 1%) and 5^{th} & 6^{th} column (2% 292 and Inulin 2%).

FIG. 3 is a graphical representation of data showing in vivo effect in rats of a diet including barley grain 292 compared to corresponding oligofructose standards and controls. The total amount of short chain fatty acids and acidification of caecal digesta was greater for treatments than controls and barley grain 292 produced comparative results to those of oligofructose standards. Items in key for SCFA types running top to bottom correspond to columns of bar graph for each treatment running from left to right.

### DETAILED DESCRIPTION

The present specification was based at least in part upon the discovery that cereal barley plants having reduced levels of synthesis of amylopectin through down regulation of starch synthase II gene expression also exhibit low levels of amylopectin, a relatively high proportion of amylose in the total starch of the grain, enhanced levels of sugars and surprisingly high levels of non-starch polysaccharide (NSP) and particularly fructan on a weight basis (see Table 1 and Table 3). Transcriptional profiles of plants comprising a loss of function mutation in SSII or SSII and exhibiting high fructan levels are shown in Table 2.

Accordingly, in one embodiment, the specification provides a method of producing a high-fructan product, wherein the method comprises: (i) obtaining or producing a cereal plant or grain or flour therefrom wherein the cereal grain or flour comprises at least 3%, preferably at least 4%, fructan as a percentage of the cereal grain or flour weight; (ii) processing the plant, grain or flour to produce the product, and optionally (iii) assessing the level or type of fructan in the cereal plant or grain or flour therefrom, or the product therefrom. It is believed that the presence of 3% or 4% fructan in the grain of cereal distinguishes the present invention from the prior art. However, illustrative levels of at least about 10% fructan are described and accordingly, the percentage fructan of the cereal grain or flour by weight in some embodiments, is at least 5%, at least 6%, at least 7%, at least 8%, at least 9% or at least 10%. If the product is isolated fructan, the product may comprise at least 50%, or at least 60% or at least 70% or preferably at least 80% fructan by weight.

In another embodiment, the method comprises: (i) obtaining or producing a cereal plant or grain or flour therefrom wherein wherein the cereal plant or grain is modified to comprise a reduced level of an endogenous polypeptide with starch synthase II (SSII) activity relative to an unmodified control; (ii) processing the plant, grain or flour to produce the product, and optionally (iii) assessing the level or type of fructan in the cereal plant or grain or flour therefrom, or the product therefrom.

It is unexpected that reduction in starch synthase activity which reduces the formation of amylopectin, also increases fructan production in the plant. Starch serves as the primary carbohydrate component in the diet of humans, in particular from cereals. Starch is the major storage carbohydrate in cereals, making up approximately 45-65% of the weight of the mature grain. However, cereal grains also contain non-starch polysaccharides such as β-glucans or low levels of fructans. In wild-type wheat grain, the level of fructan is only 0.6%-2.6% by weight.

Starch is composed only of glucosidic residues and is found as two types of molecules, amylose and amylopectin, which can be distinguished on the basis of molecular size or other properties. Amylose molecules are essentially linear polymers composed of α-1,4 linked glucosidic units, while amylopectin is a highly branched molecule with α-1,6 glucosidic bonds linking many linear chains of α-1,4 linked glucosidic units. Amylopectin is made of large molecules ranging in size between several tens of thousands to hundreds of thousands of glucose units with around 5 percent α-1,6 branches. Amylose on the other hand is composed of molecules ranging in size between several hundreds to several thousand glucosidic residues with less than one percent branches (for review see Buléon et al., International Journal of Biological Macromolecules, 23: 85-112, 1998). Wild-type cereal starches typically contain 20-30% amylose while the remainder is amylopectin.

The synthesis of starch in the endosperm of higher plants is carried out by a suite of enzymes that catalyse four key steps. Firstly, ADP-glucose pyrophosphorylase activates the monomer precursor of starch through the synthesis of ADP-glucose from G-1-P and ATP. Secondly, the activated glucosyl donor, ADP-glucose, is transferred to the non-reducing end of a pre-existing al-4 linkage by starch synthases. Thirdly, starch branching enzymes introduce branch points through the cleavage of a region of α-1,4 linked glucan followed by transfer of the cleaved chain to an acceptor chain, forming a new α-1,6 linkage. Starch branching enzymes are the only enzymes that can introduce the α-1,6 linkages into a,-polyglucans and therefore play an essential role in the formation of amylopectin. Finally, starch debranching enzymes remove some of the branch linkages although the mechanism through which they act is unresolved.

While it is clear that at least these four activities are required for normal starch granule synthesis in higher plants, multiple isoforms of each of the four activities are found in the endosperm of higher plants and specific roles have been proposed for individual isoforms on the basis of mutational analysis or through the modification of gene expression levels using transgenic approaches. In the cereal endosperm, four classes of starch synthase are found in the cereal endosperm, an isoform exclusively localised within the starch granule, granule-bound starch synthase (GBSS) which is essential for amylose synthesis, two forms that are partitioned between the granule and the soluble fraction (SSI, Li et al., Plant Physiology, 120: 1147-1155, 1999a, SSII, Li et al., Theoretical and Applied Genetics, 98: 1208-1216, 1999b) and a fourth form that is entirely located in the soluble fraction, SSIII (Cao et al., Archives of Biochemistry and Biophysics, 373: 135-146, 2000; Li et al., 1999b (supra); Li et al., Plant Physiology, 123: 613-624, 2000). Mutations in SSII and SSIII have been shown to alter amylopectin structure (Gao et al., Plant Cell, 10: 399-412, 1998; Craig et al., Plant Cell 10: 413-426, 1998). No mutations defining a role for SSI activity have been described.

Three forms of branching enzyme are expressed in the cereal endosperm, branching enzyme I (SBEI), branching enzyme IIa (SBEIIa) and branching enzyme IIb (SBEIIb) (Hedman and Boyer, Biochemical Genetics, 20: 483-492, 1982; Boyer and Preiss, Carbohydrate Research, 61: 321-334, 1978; Mizuno et al., Journal of Biochemistry, 112: 643-651, 1992; Sun et al., The New Phytologist, 137: 215-215, 1997). Alignment of SBE sequences has revealed a high degree of sequence similarity at both the nucleotide and amino acid levels and allows the grouping into the SBEI, SBEIIa and SBEIIb classes.

Two types of debranching enzymes are present in higher plants and are defined on the basis of their substrate specificities, isoamylase type debranching enzymes, and pullulanase type debranching enzymes (Myers et al., Plant Physiology, 122: 989-997, 2000). Sugary-1 mutations in maize and rice are associated with deficiency of both debranching enzymes (James et al., Plant Cell, 7: 417-429, 1995; Kubo et al., Plant Physiology, 121: 399-409, 1999) however the causal mutation maps to the same location as the isoamylase-type debranching enzyme gene.

A mutant form of barley, designated M292 or M342, has been shown to have an elevated amylose starch phenotype and a reduced amylopectin starch phenotype. This phenotype has suspected benefits for human health (Morell et al., Plant J. 34: 173-185, 2003; Topping et al., Starch/Starke 55: 539-545, 2003; Bird et al., J. Nutr. 134: 831-835, 2004a; Bird et al. Br. J. Nutr. 92: 607-615, 2004b). It is caused by a mutation in the starch synthase IIa gene (SSIIa) located on chromosome 7H of barley, as described in international patent application PCT/AU01/01452 (Publication No. WO 02/37955) the disclosure of which is incorporated herein by reference.

The barley sex6 mutation resulted from the presence of a stop codon within the starch synthase ha (SSIIa) gene. The stop codon lead to premature termination of translation of the transcript. The SSIIa protein was not detectable in the endosperm of this mutant (Morell et al. 2003 (supra)). The loss of SSIIa activity lead to an 80% decrease in amylopectin synthesis, and the remaining amylopectin polymers in general have altered chain length distribution, and consequently an altered amylose: amylopectin ratio so that the starch of the grain contained about 70% amylose.

SSII mutants of wheat have also been produced (Yamamori et al., Theor. Appl. Genet. 101: 21-29, 2000) although the amylose level was not as high as in the barley mutant, reaching about 38% in the wheat. In contrast, down-regulation of the genes encoding SBEIIa in wheat resulted in a high amylose phenotype, with about 80% amylose in the starch of the grain (Regina et al., Proc. Natl. Acad. Sci. U.S.A. 103: 3546-3551, 2006).

In some embodiments, the present invention provides for improvements in cereal plant utility by increasing the level of fructan in grain. The modification may be limited to grain or alternatively, the modification may be thoughout the plant in various of its tissues and parts. As used herein, “modifying” or “modified” means a change in the plant or grain, which may be an increase or decrease in amount, activity, rate of production, rate of inactivation, rate of breakdown, delay of onset, earlier onset, addition or removal of material, mutation, or any combination of these, so long as there is a reduced level or activity of starch synthase II. The terms include either an increase or decrease in the functional level of a gene or protein of interest. “Functional level” should be understood to refer to the level of active protein. The functional level is a combination of the actual level of protein present in the host cell and the specific activity of the protein. Accordingly, the functional level may e.g. be modified by increasing or decreasing the actual protein concentration in the host cell, which may readily be achieved by altering expression of a gene encoding the protein. The functional level may also be modified by modulating the specific activity of the protein. Such increase or decrease of the specific activity may be achieved by expressing a variant protein with higher or lower specific activity or by replacing the endogenous gene encoding the relevant protein with an allele encoding such a variant. Increase or decrease of the specific activity may also be achieved by expression of an effector molecule. In certain embodiments, the expression level of an appropriate coding sequence or activity or amount of an enzyme is chosen such that it is at least about 10%, at least 20%, at least 30%, at least 40%, at least 50%, at least 60%, at least 80% or even at least about 100%, at least 200%, at least 500%, or at least 1000% higher, or at least about 10%, at least 20%, at least 30%, at least 40%, at least 50%, at least 60%, at least 70%, at least 80%, at least 90%, at least 92%, at least 94%, at least 96%, at least 97%, at least 98% or at least 99% lower than a reference expression level, or reduced to an undetectable level.

Another way of distinguishing the required reduction in SSII level or activity is by quantifying the increase level or the increase in various forms of fuctan in a modified plant or grain therefrom. As used herein, the terms “modifying”, “altering”, “increasing”, “increased”, “reducing”, “reduced”, “inhibited”, “mutant” or the like are considered relative terms, i.e. in comparison with the wild-type or unaltered or control state. In some embodiments, a wild-type plant is an appropriate “control plant” however in many situations the control plant must be determined by the skilled addressee using their ordinary skill in the art. The “level of a protein” refers to the amount of a particular protein, for example SSII, which may be measured by any means known in the art such as, for example, Western blot analysis or other immunological means. The “level of an enzyme activity” refers to the amount of a particular enzyme measured in an enzyme assay.

It would be appreciated that the level of activity of an enzyme might be altered in a mutant if a more or less active protein is produced, but not the expression level (amount) of the protein itself. Conversely, the amount of protein might be altered but the activity (per unit protein) remain the same. Reductions in both amount and activity are also possible such as, for example, when the expression of a gene encoding the enzyme is reduced transcriptionally or post-transcriptionally. In certain embodiments, the reduction in the level of protein or activity of SSII is by at least 40% or by at least 60% compared to the level of protein or activity in the grain of unmodified cereal, for example wheat or barley, or by at least 75%, at least 90% or at least 95%. The reduction in the level of the protein or enzyme activity or gene expression may occur at any stage in the development of the leaf, seed or grain, particularly during the daytime when photosynthesis is occurring, or during the grain filling stage while starch is being synthesized in the developing endosperm, or at all stages of grain development through to maturity. The term “wild-type” as used herein has its normal meaning in the field of genetics and includes plant, preferably cereal, cultivars or genotypes which are not modified as taught herein. Some preferred “wild-type” cereal plant varieties are described herein.

The modified phenotype may be achieved by partial or full inhibition of the expression of an SSII gene. Techniques well known in the art such as SDS-PAGE and immunoblotting are carried out on hydrolysed and unhydrolysed grains and fractions thereof to identify the plants or grain where modifications have occurred to starch forming enzymes, carbohydrate related genes, defense related genes, stress protein related genes or genes identified as differentially expressed in the subject modified plants or grain thereform (such as those listed in Table 2). These methods include analysis of plants by methods described herein or further by methods such as such as microarray analysis, electrophoresis, chromatography (including paper chromatography, thin layer chromatography, gas chromatography, gas-liquid chromatography and high-performance liquid chromatography) techniques. Separated components are typically identified by comparison of separation profiles with standards of known identity, or by analytical techniques such as mass spectrometry and nuclear magnetic resonance spectroscopy. For example, reference may be made to Example 9, Robinson, The Organic Constituents of Higher Plants, Cordus Press, North Amherst, USA, 1980; Adams et al., Anal. Biochem., 266: 77-84, 1999; Veronese et al., Enz. Microbial Tech., 24: 263-269, 1999; Hendrix et al., J. Insect Physiol., 47: 423-432, 2001; Thompson et al., Carbohydrate Res., 331: 149-161, 2001; and references cited therein. Carbohydrates can be assayed using standard protocols known to persons skilled in the art.

Alteration in SSII activities may be achieved by the introduction of one or more genetic variations into the cereal plant. That is, the genetic variations lead, directly or indirectly, to the alteration in enzyme activity in the plant part during growth or development and consequently to the enzyme and fructan modifications described herein. The genetic variation may be a heterologous polynucleotide which is introduced into the plant or a progenitor cell, for example by transformation or mutagenesis. The genetic variation may subsequently be introduced into different genetic backgrounds by crossing, as known in the art of plant breeding. In some embodiments, the level or functional activity of SSII is down regulated to a level less than about 80%, less than 70%, less than 60%, less than 50%, less than 40%, less than 30%, less than 20% or less than 15%, and suitably less than about 10%, less than 9%, less than 8%, less than 7%, less than 6%, less than 5%, less than 4%, less than 3%, less than 2% or less than 1% relative to a corresponding control plant to achieve elevated levels of fructan. In a preferred embodiment, elevated levels are at least twice that of controls. Preferably, in this embodiment, this reduction results in a substantial enhancement of non-starch polysaccharide such as fructan levels which is generally at least about 50% or 55% and more especially at least about 60%, at least 65%, at least 70%, at least 75%, at least 80%, at least 85%, at least 90%, at least 95% or greater increase in fructan level relative to a corresponding control plant grown under the same environmental conditions. The amount of reduced SSII level or activity required may depend upon other factors such as the plant species or strain environmental factors. However, it is considered that any optimisation, which may be required in such an event is achievable using routine methods including those described herein.

Reduced SSII levels may be accomplished in tissues throughout the plant, for example using a constitutive promoter to drive expression of a heterologous polynucleotide that down regulates SSII. Alternatively, it may be accomplished in source tissues (leaves), in transport tissues or in sink tissues (endosperm) using a tissue-specific or developmentally regulated promoter. “Sink cell” and “sink tissue” as used herein, refer to cells, tissues or organs which comprise a net inflow of organic carbon that has entered the cells in a form other than fixation of carbon dioxide ie. as sugars or other carbohydrates. In plants, sink tissues include all non-photosynthetic tissues, as well as photosynthetic tissues with a net inflow of organic carbon fixed by other photosynthetic cells or otherwise obtained from the surrounding medium or environment by means other than direct fixation of carbon dioxide.

In certain embodiments, the level fructan in grain is increased at least about 10%, at least 20%, at least 30%, at least 40%, at least 50%, at least 60%, at least 70%, at least 80% or at least 90%, or even at least about 100%, at least 200%, at least 300%, at least 400%, at least 500%, at least 600%, at least 700%, at least 800%, at least 900% or at least 1000% higher relative to controls.

### Genes

In some embodiments, the present invention involves modification of gene activity and the construction and use of chimeric genes. As used herein, the term “gene” includes any deoxyribonucleotide sequence which includes a protein coding region or which is transcribed in a cell but not translated, as well as associated non-coding and regulatory regions. Such associated regions are typically located adjacent to the coding region or the transcribed region on both the 5′ and 3′ ends for a distance of about 2 kb on either side. In this regard, the gene may include control signals such as promoters, enhancers, termination and/or polyadenylation signals that are naturally associated with a given gene, or heterologous control signals in which case the gene is referred to as a “chimeric gene”. The sequences which are located 5′ of the coding region and which are present on the mRNA are referred to as 5′ non-translated sequences. The sequences which are located 3′ or downstream of the coding region and which are present on the mRNA are referred to as 3′ non-translated sequences. The term “gene” encompasses both cDNA and genomic forms of a gene.

The “starch synthase II gene” “SSII” or the like as used herein refers to a nucleotide sequence encoding starch synthase II (SSII) in cereals such as barley or wheat, which can readily be distinguished from other starch synthases or other proteins by those skilled in the art. Wheat SSII genes include the naturally occurring variants existing in wheat, including those encoded by the A, B and D genomes of breadwheat, as well as non-naturally occurring variants which may be produced by those skilled in the art of gene modification. In a preferred embodiment, a barley SSII gene refers to a nucleic acid molecule, which may be present in or isolated from barley or derived therefrom, comprising nucleotides having a sequence having at least 80% identity to the cDNA sequence shown in SEQ ID NO: 1. In another preferred embodiment, a wheat SSII gene refers to a nucleic acid molecule, which may be present in or isolated from wheat or derived therefrom, comprising nucleotides having a sequence having at least 80% identity to the cDNA shown in SEQ ID NO: 3, 5, 7, 9 or 18. In a preferred embodiment, the SSII gene is an SSIIa gene, or the SSII protein is an SSIIa protein, each of which may be applied to any or all of the aspects of the invention disclosed herein.

A genomic form or clone of a gene containing the transcribed region may be interrupted with non-coding sequences termed “introns” or “intervening regions” or “intervening sequences.” An “intron” as used herein is a segment of a gene which is transcribed as part of a primary RNA transcript but is not present in the mature mRNA molecule. Introns are removed or “spliced out” from the nuclear or primary transcript; introns therefore are absent in the messenger RNA (mRNA). Introns may contain regulatory elements such as enhancers. “Exons” as used herein refer to the DNA regions corresponding to the RNA sequences which are present in the mature mRNA or the mature RNA molecule in cases where the RNA molecule is not translated. An mRNA functions during translation to specify the sequence or order of amino acids in a nascent polypeptide. The term “gene” includes a synthetic or fusion molecule encoding all or part of the proteins of the invention described herein and a complementary nucleotide sequence to any one of the above. A gene may be introduced into an appropriate vector for extrachromosomal maintenance in a cell or for integration into the host genome.

As used herein, a “chimeric gene” refers to any gene that is not a native gene in its native location. Typically a chimeric gene comprises regulatory and transcribed or protein coding sequences that are not found together in nature. Accordingly, a chimeric gene may comprise regulatory sequences and coding sequences that are derived from different sources, or regulatory sequences and coding sequences derived from the same source, but arranged in a manner different than that found in nature. The term “endogenous” is used herein to refer to a substance that is normally present or produced in an unmodified plant at the same developmental stage as the plant under investigation. An “endogenous gene” refers to a native gene in its natural location in the genome of an organism. As used herein, “recombinant nucleic acid molecule” refers to a nucleic acid molecule which has been constructed or modified by recombinant DNA technology. The terms “foreign polynucleotide” or “exogenous polynucleotide” or “heterologous polynucleotide” and the like refer to any nucleic acid which is introduced into the genome of a cell by experimental manipulations. These include gene sequences found in that cell so long as the introduced gene contains some modification (e.g. a mutation, the presence of a selectable marker gene, etc.) relative to the naturally-occurring gene. Foreign or exogenous genes may be genes that are inserted into a non-native organism, native genes introduced into a new location within the native host, or chimeric genes. A “transgene” is a gene that has been introduced into the genome by a transformation procedure. The term “genetically modified” includes introducing genes into cells by transformation or transduction, mutating genes in cells and altering or modulating the regulation of a gene in a cell or organisms to which these acts have been done or their progeny.

### Polynucleotides

The present invention including the description, tables and sequence listing, refers to various polynucleotides. As used herein, a “polynucleotide” or “nucleic acid” or “nucleic acid molecule” means a polymer of nucleotides, which may be DNA or RNA or a combination thereof, and includes mRNA, cRNA, cDNA, tRNA, siRNA, shRNA and hpRNA. It may be DNA or RNA of cellular, genomic or synthetic origin, for example made on an automated synthesizer, and may be combined with carbohydrate, lipids, protein or other materials, labelled with fluorescent or other groups, or attached to a solid support to perform a particular activity defined herein, or comprise one or more modified nucleotides not found in nature, well known to those skilled in the art. The polymer may be single-stranded, essentially double-stranded or partly double-stranded. An example of a partly-double stranded RNA molecule is a hairpin RNA (hpRNA), short hairpin RNA (shRNA) or self-complementary RNA which include a double stranded stem formed by basepairing between a nucleotide sequence and its complement and a loop sequence which covalently joins the nucleotide sequence and its complement. Basepairing as used herein refers to standard basepairing between nucleotides, including G:U basepairs. “Complementary” means two polynucleotides are capable of basepairing (hybridizing) along part of their lengths, or along the full length of one or both. A “hybridized polynucleotide” means the polynucleotide is actually basepaired to its complement. The term “polynucleotide” is used interchangeably herein with the term “nucleic acid”.

By “isolated” is meant material that is substantially or essentially free from components that normally accompany it in its native state. As used herein, an “isolated polynucleotide” or “isolated nucleic acid molecule” means a polynucleotide which is at least partially separated from, preferably substantially or essentially free of, the polynucleotide sequences of the same type with which it is associated or linked in its native state. For example, an “isolated polynucleotide” includes a polynucleotide which has been purified or separated from the sequences which flank it in a naturally occurring state, e.g., a DNA fragment which has been removed from the sequences which are normally adjacent to the fragment. Preferably, the isolated polynucleotide is also at least 90% free from other components such as proteins, carbohydrates, lipids etc. The term “recombinant polynucleotide” as used herein refers to a polynucleotide formed in vitro by the manipulation of nucleic acid into a form not normally found in nature. For example, the recombinant polynucleotide may be in the form of an expression vector. Generally, such expression vectors include transcriptional and translational regulatory nucleic acid operably connected to the nucleotide sequence.

The present invention refers to use of oligonucleotides. As used herein, “oligonucleotides” are polynucleotides up to 50 nucleotides in length. They can be RNA, DNA, or combinations or derivatives of either. Oligonucleotides are typically relatively short single stranded molecules of 10 to 30 nucleotides, commonly 15-25 nucleotides in length. When used as a probe or as a primer in an amplification reaction, the minimum size of such an oligonucleotide is the size required for the formation of a stable hybrid between the oligonucleotide and a complementary sequence on a target nucleic acid molecule. Preferably, the oligonucleotides are at least 15 nucleotides, more preferably at least 18 nucleotides, more preferably at least 19 nucleotides, more preferably at least 20 nucleotides, even more preferably at least 25 nucleotides in length.

Polynucleotides used as a probe are typically conjugated with a detectable label such as a radioisotope, hapten, an enzyme, biotin, a fluorescent molecule or a chemiluminescent molecule. Oligonucleotides of the invention are useful in methods of detecting an allele of an SSII or other gene linked to a trait of interest, for example modified starch or fructan levels. Such methods, for example, employ nucleic acid hybridization and in many instances include oligonucleotide primer extension by a suitable polymerase (as used in PCR).

A variant of an oligonucleotide of the invention includes molecules of varying sizes of, and/or are capable of hybridising, for example, to the cereal genome close to that of, the specific oligonucleotide molecules defined herein. For example, variants may comprise additional nucleotides (such as 1, 2, 3, 4, or more), or less nucleotides as long as they still hybridise to the target region. Furthermore, a few nucleotides may be substituted without negatively influencing the ability of the oligonucleotide to hybridise to the target region. In addition, variants may readily be designed which hybridise close to, for example to within 50 nucleotides, the region of the plant genome where the specific oligonucleotides defined herein hybridise. Probes, oligonucleotides and the like are based upon the herein described sequences or corrected versions thereof or variants thereof or functional homologs from other cereal plants.

The terms “polynucleotide variant” and “variant” and the like refer to polynucleotides or their complementary forms displaying substantial sequence identity with a reference polynucleotide sequence. These terms also encompass polynucleotides that are distinguished from a reference polynucleotide by the addition, deletion or substitution of at least one nucleotide. Accordingly, the terms “polynucleotide variant” and “variant” include polynucleotides in which one or more nucleotides have been added or deleted, or replaced with different nucleotides. In this regard, it is well understood in the art that certain alterations inclusive of mutations, additions, deletions and substitutions can be made to a reference polynucleotide whereby the altered polynucleotide retains the biological function or activity of the reference polynucleotide. Accordingly, these terms encompass polynucleotides that encode polypeptides that exhibit enzymatic or other regulatory activity, or polynucleotides capable of serving as selective probes or other hybridising agents. In particular, this includes polynucleotides which encode the same polypeptide or amino acid sequence but which vary in nucleotide sequence by redundancy of the genetic code. The terms “polynucleotide variant” and “variant” also include naturally occurring allelic variants.

By “corresponds to” or “corresponding to” is meant a polynucleotide (a) having a nucleotide sequence that is substantially identical or complementary to all or most of a reference polynucleotide sequence or (b) encoding an amino acid sequence identical to an amino acid sequence in a peptide or protein. This phrase also includes within its scope a peptide or polypeptide having an amino acid sequence that is substantially identical to a sequence of amino acids in a reference peptide or protein. Terms used to describe sequence relationships between two or more polynucleotides or polypeptides include “reference sequence”, “comparison window”, “sequence identity”, “percentage of sequence identity”, “substantial identity” and “identical”, and are defined with respect to a minimum number of nucleotides or amino acid residues or over the full length. The terms “sequence identity” and “identity” are used interchangeably herein to refer to the extent that sequences are identical on a nucleotide-by-nucleotide basis or an amino acid-by-amino acid basis over a window of comparison. Thus, a “percentage of sequence identity” is calculated by comparing two optimally aligned sequences over the window of comparison, determining the number of positions at which the identical nucleic acid base (e.g., A, T, C, G, U) or the identical amino acid residue (e.g., Ala, Pro, Ser, Thr, Gly, Val, Leu, Ile, Phe, Tyr, Trp, Lys, Arg, His, Asp, Glu, Asn, Gln, Cys and Met) occurs in both sequences to yield the number of matched positions, dividing the number of matched positions by the total number of positions in the window of comparison (i.e., the window size), and multiplying the result by 100 to yield the percentage of sequence identity.

The % identity of a polynucleotide can be determined by GAP (Needleman and Wunsch, J. Mol. Biol. 48: 443-453, 1970) analysis (GCG program) with a gap creation penalty=5, and a gap extension penalty=0.3. Unless stated otherwise, the query sequence is at least 45 nucleotides in length, and the GAP analysis aligns the two sequences over a region of at least 45 nucleotides. Preferably, the query sequence is at least 150 nucleotides in length, and the GAP analysis aligns the two sequences over a region of at least 150 nucleotides. More preferably, the query sequence is at least 300 nucleotides in length and the GAP analysis aligns the two sequences over a region of at least 300 nucleotides, or at least 400, at least 500 or at least 600 nucleotides in each case. Reference also may be made to the BLAST family of programs as for example disclosed by Altschul et al., Nucleic Acids Res. 25: 3389, 1997. A detailed discussion of sequence analysis can be found in Unit 19.3 of Ausubel et al., “Current Protocols in Molecular Biology”, John Wiley & Sons Inc, 1994-1998, Chapter 15.

Nucleotide or amino acid sequences are indicated as “essentially similar” when such sequences have a sequence identity of at least 80%, particularly at least 85%, quite particularly at least 90%, especially at least 95%, more especially are identical. It is clear that when RNA sequences are described as essentially similar to, correspond to, or have a certain degree of sequence identity with, DNA sequences, thymine (T) in the DNA sequence is considered equal to uracil (U) in the RNA sequence.

With regard to the defined polynucleotides, it will be appreciated that % identity figures higher than those provided above will encompass preferred embodiments. Thus, where applicable, in light of the minimum % identity figures, it is preferred that the polynucleotide comprises a polynucleotide sequence which is at least 80%, more preferably at least 85%, more preferably at least 90%, more preferably at least 91%, more preferably at least 92%, more preferably at least 93%, more preferably at least 94%, more preferably at least 95%, more preferably at least 96%, more preferably at least 97%, more preferably at least 98%, more preferably at least 99%, more preferably at least 99.1%, more preferably at least 99.2%, more preferably at least 99.3%, more preferably at least 99.4%, more preferably at least 99.5%, more preferably at least 99.6%, more preferably at least 99.7%, more preferably at least 99.8%, and even more preferably at least 99.9% identical to the relevant nominated SEQ ID NO.

Preferably, a polynucleotide of the invention which encodes a polypeptide with SSII activity is greater than 800, preferably greater than 900, and even more preferably greater than 1,000 or 2000 nucleotides in length.

Polynucleotides of the present invention may possess, when compared to naturally occurring molecules, one or more mutations which are deletions, insertions, or substitutions of nucleotide residues. Mutants can be either naturally occurring (that is to say, isolated from a natural source) or synthetic (for example, by performing site-directed mutagenesis on the nucleic acid).

The present invention refers to the stringency of hybridization conditions to define the extent of complementarity of two polynucleotides. “Stringency” as used herein, refers to the temperature and ionic strength conditions, and presence or absence of certain organic solvents, during hybridization and washing. The higher the stringency, the higher will be the degree of complementarity between a target nucleotide sequence and the labelled polynucleotide sequence (probe). “Stringent conditions” refers to temperature and ionic conditions under which only nucleotide sequences having a high frequency of complementary bases will hybridize. As used herein, the term “hybridizes under low stringency, medium stringency, high stringency, or very high stringency conditions” describes conditions for hybridization and washing. Guidance for performing hybridization reactions can be found in Ausubel et al., (eds.), Current Protocols in Molecular Biology, John Wiley & Sons, NY, 6.3.1-6.3.6., 1989. Aqueous and nonaqueous methods are described in that reference and either can be used. Specific hybridization conditions referred to herein are as follows: 1) low stringency hybridization conditions are for hybridization in 6× sodium chloride/sodium citrate (SSC) at 45° C., followed by two washes in 0.2×SSC, 0.1% SDS at 50-55° C.; 2) medium stringency hybridization conditions are for hybridization in 6×SSC at about 45° C., followed by one or more washes in 0.2×SSC, 0.1% SDS at 60° C.; 3) high stringency hybridization conditions are for hybridization in 6×SSC at 45° C., followed by one or more washes in 0.2×SSC, 0.1% SDS at 65° C.; and 4) very high stringency hybridization conditions are for hybridization in 0.5 M sodium phosphate buffer, 7% SDS at 65° C., followed by one or more washes at 0.2×SSC, 1% SDS at 65° C.

### Polypeptides

The terms “polypeptide” and “protein” are generally used interchangeably. The terms “proteins” and “polypeptides” as used herein also include variants, mutants, modifications, analogs and/or derivatives of the polypeptides of the invention as described herein. As used herein, “substantially purified polypeptide” refers to a polypeptide that has been separated from the lipids, nucleic acids, other peptides and other molecules with which it is associated in its native state. Preferably, the substantially purified polypeptide is at least 90% free from other components with which it is naturally associated. By “recombinant polypeptide” is meant a polypeptide made using recombinant techniques, i.e., through the expression of a recombinant polynucleotide in a cell, preferably a plant cell and more preferably a cereal plant cell.

Illustrative polypeptides having SSII activity are set out in the sequence listing and described in Table 7. Accordingly, the present invention proposes without limitation the modification of SSII polypeptides having the amino acid sequences set forth in SEQ ID NO: 2, 4, 6, 8, 11, 13, 17, 19, 21, and 23 and naturally occurring variants, corrected versions thereof and variants as described herein such as variants having about 80% sequence identity.

With regard to a defined polypeptide, it will be appreciated that % identity figures higher than those provided above will encompass preferred embodiments. Thus, where applicable, in light of the minimum % identity figures, it is preferred that the polypeptide comprises an amino acid sequence which is at least 75%, more preferably at least 80%, more preferably at least 85%, more preferably at least 90%, more preferably at least 91%, more preferably at least 92%, more preferably at least 93%, more preferably at least 94%, more preferably at least 95%, more preferably at least 96%, more preferably at least 97%, more preferably at least 98%, more preferably at least 99%, more preferably at least 99.1%, more preferably at least 99.2%, more preferably at least 99.3%, more preferably at least 99.4%, more preferably at least 99.5%, more preferably at least 99.6%, more preferably at least 99.7%, more preferably at least 99.8%, and even more preferably at least 99.9% identical to the relevant nominated SEQ ID NO.

The % identity of a polypeptide relative to another polypeptide can be determined by GAP (Needleman and Wunsch, 1970 (supra)) analysis (GCG program) with a gap creation penalty=5, and a gap extension penalty=0.3. The query sequence is at least 15 amino acids in length, and the GAP analysis aligns the two sequences over a region of at least 15 amino acids. More preferably, the query sequence is at least 50 amino acids in length, and the GAP analysis aligns the two sequences over a region of at least 50 amino acids. More preferably, the query sequence is at least 100 amino acids in length and the GAP analysis aligns the two sequences over a region of at least 100 amino acids. Even more preferably, the query sequence is at least 250 amino acids in length and the GAP analysis aligns the two sequences over a region of at least 250 amino acids.

As used herein a “biologically active” fragment of a polypeptide is a portion of a polypeptide of the invention, less than full length, which maintains a defined activity of the full-length polypeptide. In a particularly preferred embodiment, the biologically active fragment is able to synthesize starch to produce amylose chains having a DP of at least 15. Biologically active fragments can be any size as long as they maintain the defined activity, but are preferably at least 200 or at least 250 amino acid residues long.

With regard to a defined polypeptide, it will be appreciated that % identity figures higher than those provided above will encompass preferred embodiments. Thus, where applicable, in light of the minimum % identity figures, it is preferred that the polypeptide comprises an amino acid sequence which is at least 80%, more preferably at least 85%, more preferably at least 90%, more preferably at least 91%, more preferably at least 92%, more preferably at least 93%, more preferably at least 94%, more preferably at least 95%, more preferably at least 96%, more preferably at least 97%, more preferably at least 98%, more preferably at least 99%, more preferably at least 99.1%, more preferably at least 99.2%, more preferably at least 99.3%, more preferably at least 99.4%, more preferably at least 99.5%, more preferably at least 99.6%, more preferably at least 99.7%, more preferably at least 99.8%, and even more preferably at least 99.9% identical to the relevant nominated SEQ ID NO.

Amino acid sequence mutants of the polypeptides of the present invention can be prepared by introducing appropriate nucleotide changes into a nucleic acid of the present invention, or by in vitro synthesis of the desired polypeptide. Such mutants include, for example, deletions, insertions or substitutions of residues within the amino acid sequence. A combination of deletion, insertion and substitution can be made to arrive at the final construct, provided that the final peptide product possesses the desired characteristics.

Mutant (altered) peptides can be prepared using any technique known in the art. For example, a polynucleotide of the invention can be subjected to in vitro mutagenesis. Such in vitro mutagenesis techniques include sub-cloning the polynucleotide into a suitable vector, transforming the vector into a “mutator” strain such as the E. coli× L-1 red (Stratagene) and propagating the transformed bacteria for a suitable number of generations. In another example, the polynucleotides of the invention are subjected to DNA shuffling techniques as broadly described by Harayama, Trends Biotechnol. 16: 76-82, 1998. These DNA shuffling techniques may include genes related to those of the present invention, such as SSII genes from plant species other than wheat or barley, and/or include different genes from the same plant encoding similar proteins, such as the wheat or barley starch synthase I or III genes. Products derived from mutated/altered DNA can readily be screened using techniques described herein to determine if they possess, for example, starch synthase activity.

In designing amino acid sequence mutants, the location of the mutation site and the nature of the mutation will depend on characteristic(s) to be modified. The sites for mutation can be modified individually or in series, e.g., by (1) substituting first with conservative amino acid choices and then with more radical selections depending upon the results achieved, (2) deleting the target residue, or (3) inserting other residues adjacent to the located site.

Amino acid sequence deletions generally range from about 1 to 15 residues, more preferably about 1 to 10 residues and typically about 1 to 5 contiguous residues.

Substitution mutants have at least one amino acid residue in the polypeptide molecule removed and a different residue inserted in its place. The sites of greatest interest for substitutional mutagenesis include sites identified as the active site(s). Other sites of interest are those in which particular residues obtained from various strains or species are identical. These positions may be important for biological activity. These sites, especially those falling within a sequence of at least three other identically conserved sites, are preferably substituted in a relatively conservative manner. Such conservative substitutions are shown in Table 9 under the heading of “exemplary substitutions”.

Polypeptides of the present invention can be produced in a variety of ways, including production and recovery of natural polypeptides, production and recovery of recombinant polypeptides, and chemical synthesis of the polypeptides. In one embodiment, an isolated polypeptide of the present invention is produced by culturing a cell capable of expressing the polypeptide under conditions effective to produce the polypeptide, and recovering the polypeptide. A preferred cell to culture is a recombinant cell of the present invention. Effective culture conditions include, but are not limited to, effective media, bioreactor, temperature, pH and oxygen conditions that permit polypeptide production. An effective medium refers to any medium in which a cell is cultured to produce a polypeptide of the present invention. Such medium typically comprises an aqueous medium having assimilable carbon, nitrogen and phosphate sources, and appropriate salts, minerals, metals and other nutrients, such as vitamins. Cells of the present invention can be cultured in conventional fermentation bioreactors, shake flasks, test tubes, microtiter dishes, and petri plates. Culturing can be carried out at a temperature, pH and oxygen content appropriate for a recombinant cell. Such culturing conditions are within the expertise of one of ordinary skill in the art.

The present invention refers to elements which are operably connected or linked. “Operably connected” or “operably linked” and the like refer to a linkage of polynucleotide elements in a functional relationship. Typically, operably connected nucleic acid sequences are contiguously linked and, where necessary to join two protein coding regions, contiguous and in reading frame. A coding sequence is “operably connected to” another coding sequence when RNA polymerase will transcribe the two coding sequences into a single RNA, which if translated is then translated into a single polypeptide having amino acids derived from both coding sequences. The coding sequences need not be contiguous to one another so long as the expressed sequences are ultimately processed to produce the desired protein.

As used herein, the term “cis-acting sequence”, “cis-acting element” or “cis-regulatory region” or “regulatory region” or similar term shall be taken to mean any sequence of nucleotides, which when positioned appropriately and connected relative to an expressible genetic sequence, is capable of regulating, at least in part, the expression of the genetic sequence. Those skilled in the art will be aware that a cis-regulatory region may be capable of activating, silencing, enhancing, repressing or otherwise altering the level of expression and/or cell-type-specificity and/or developmental specificity of a gene sequence at the transcriptional or post-transcriptional level. In certain embodiments of the present invention, the cis-acting sequence is an activator sequence that enhances or stimulates the expression of an expressible genetic sequence.

“Operably connecting” a promoter or enhancer element to a transcribable polynucleotide means placing the transcribable polynucleotide (e.g., protein-encoding polynucleotide or other transcript) under the regulatory control of a promoter, which then controls the transcription of that polynucleotide. In the construction of heterologous promoter/structural gene combinations, it is generally preferred to position a promoter or variant thereof at a distance from the transcription start site of the transcribable polynucleotide which is approximately the same as the distance between that promoter and the protein coding region it controls in its natural setting; i.e., the gene from which the promoter is derived. As is known in the art, some variation in this distance can be accommodated without loss of function. Similarly, the preferred positioning of a regulatory sequence element (e.g., an operator, enhancer etc) with respect to a transcribable polynucleotide to be placed under its control is defined by the positioning of the element in its natural setting; i.e., the genes from which it is derived. [00100]“Promoter” or “promoter sequence” as used herein refers to a region of a gene, generally upstream (5′) of the RNA encoding region, which controls the initiation and level of transcription in the cell of interest. A “promoter” includes the transcriptional regulatory sequences of a classical genomic gene, including a TATA box and CCAAT box sequences, as well as additional regulatory elements (i.e., upstream activating sequences, enhancers and silencers) that alter gene expression in response to developmental and/or environmental stimuli, or in a tissue-specific or cell-type-specific manner. A promoter is usually, but not necessarily (for example, some PoIIII promoters), positioned upstream of a structural gene, the expression of which it regulates. Furthermore, the regulatory elements comprising a promoter are usually positioned within 2 kb of the start site of transcription of the gene. Promoters may contain additional specific regulatory elements, located more distal to the start site to further enhance expression in a cell, and/or to alter the timing or inducibility of expression of a structural gene to which it is operably connected.

“Constitutive promoter” refers to a promoter that directs expression of an operably linked transcribed sequence in many or all tissues of a plant. The term constitutive as used herein does not necessarily indicate that a gene is expressed at the same level in all cell types, but that the gene is expressed in a wide range of cell types, although some variation in level is often detectable. “Selective expression” as used herein refers to expression almost exclusively in specific organs of the plant, such as, for example, endosperm, embryo, leaves, fruit, tubers or root. In one embodiment, a promoter is expressed in all photosynthetic tissue, which may correspond to all aerial parts of the plant, for example a promoter that is involved in expressing a gene required for photosynthesis such as rubisco small subunit promoters. The term may also refer to expression at specific developmental stages in an organ, such as in early or late embryogenesis or different stages of maturity; or to expression that is inducible by certain environmental conditions or treatments. Selective expression may therefore be contrasted with constitutive expression, which refers to expression in many or all tissues of a plant under most or all of the conditions experienced by the plant.

Selective expression may also result in compartmentation of the products of gene expression in specific plant tissues, organs or developmental stages. Compartmentation in specific subcellular locations such as the endosperm, cytosol, vacuole, or apoplastic space may be achieved by the inclusion in the structure of the gene product of appropriate signals for transport to the required cellular compartment, or in the case of the semi-autonomous organelles (plastids and mitochondria) by integration of the transgene with appropriate regulatory sequences directly into the organelle genome.

A “tissue-specific promoter” or “organ-specific promoter” is a promoter that is preferentially expressed in one tissue or organ relative to many other tissues or organs, preferably most if not all other tissues or organs in a plant. Typically, the promoter is expressed at a level 10-fold higher in the specific tissue or organ than in other tissues or organs. An illustrative tissue specific promoter is the promoter for high molecular weight (HMW) glutenin gene, Bx17 which is expressed preferentially in the developing endosperm of cereal plants. Further endosperm specific promoters include the high molecular weight glutenin promoter, the wheat SSI promoter, and the wheat BEII promoter.

The promoters contemplated by the present invention may be native to the host plant to be transformed or may be derived from an alternative source, where the region is functional in the host plant. Other sources include the Agrobacterium T-DNA genes, such as the promoters of genes for the biosynthesis of nopaline, octapine, mannopine, or other opine promoters; promoters from plants, such as ubiquitin promoters such as the Ubi promoter from the maize ubi-1 gene, Christensen et al., Transgen. Res., 5: 213-218, 1996 (see, e.g., U.S. Pat. No. 4,962,028) or actin promoters; tissue specific promoters (see, e.g., U.S. Pat. No. 5,459,252 to Conkling et al.; WO 91/13992 to Advanced Technologies); promoters from viruses (including host specific viruses), or partially or wholly synthetic promoters. Numerous promoters that are functional in mono- and dicotyledonous plants are well known in the art (see, for example, Greve, J. Mol. Appl. Genet., 1: 499-511, 1983; Salomon et al., EMBO J., 3: 141-146, 1984; Garfinkel et al., Cell, 27: 143-153, 1983; Barker et al., Plant Mol. Biol., 2: 235-350, 1983; including various promoters isolated from plants and viruses such as the cauliflower mosaic virus promoter (CaMV 35S, 19S). Many tissue specific promoter regions are known. Other transcriptional initiation regions which preferentially provide for transcription in certain tissues or under certain growth conditions, include those from genes encoding napin, seed ACP, zein, or other seed storage proteins. Fruit specific promoters are also known, one such promoter is the E8 promoter, described by Deikman et al., EMBO J., 2: 3315-3320, 1998 and DellaPenna et al., Plant Cell, 1: 53-63, 1989. Non-limiting methods for assessing promoter activity are disclosed by Medberry et al., Plant Cell, 4: 185-192, 1992; Medberry et al., Plant J. 3: 619-626, 1993, Sambrook et al., Molecular Cloning: A Laboratory Manual (2nd ed.). Cold Spring Harbour Laboratory, Cold Spring Harbour, NY, 1989, and McPherson et al. (U.S. Pat. No. 5,164,316).

Alternatively or additionally, the promoter may be an inducible promoter or a developmentally regulated promoter which is capable of driving expression of the introduced polynucleotide at an appropriate developmental stage of the plant. Other cis-acting sequences which may be employed include transcriptional and/or translational enhancers. Enhancer regions are well known to persons skilled in the art, and can include an ATG translational initiation codon and adjacent sequences. The initiation codon must be in phase with the reading frame of the coding sequence relating to the foreign or exogenous polynucleotide to ensure translation of the entire sequence. The translation control signals and initiation codons can be of a variety of origins, both natural and synthetic. Translational initiation regions may be provided from the source of the transcriptional initiation region, or from a foreign or exogenous polynucleotide. The sequence can also be derived from the source of the promoter selected to drive transcription, and can be specifically modified so as to increase translation of the mRNA.

The nucleic acid construct of the present invention typically comprises a 3′ non-translated sequence from about 50 to 1,000 nucleotide base pairs which may include a transcription termination sequence. A 3′ non-translated sequence may contain a transcription termination signal which may or may not include a polyadenylation signal and any other regulatory signals capable of effecting mRNA processing. A polyadenylation signal is characterized by effecting the addition of polyadenylic acid tracts to the 3′ end of the mRNA precursor. Polyadenylation signals are commonly recognized by the presence of homology to the canonical form 5′ AATAAA-3′ although variations are not uncommon. Transcription termination sequences which do not include a polyadenylation signal include terminators for Poll or PoIII RNA polymerase which comprise a run of four or more thymidines. Examples of suitable 3′ non-translated sequences are the 3′ transcribed non-translated regions containing a polyadenylation signal from the nopaline synthase (nos) gene of Agrobacterium tumefaciens (Bevan et al., Nucl. Acid Res., 11: 369, 1983) and the terminator for the T7 transcript from the octopine synthase gene of Agrobacterium tumefaciens. Alternatively, suitable 3′ non-translated sequences may be derived from plant genes such as the 3′ end of the protease inhibitor I or II genes from potato or tomato, the soybean storage protein genes and the small subunit of the ribulose-1,5-bisphosphate carboxylase (ssRUBISCO) gene, although other 3′ elements known to those of skill in the art can also be employed. Alternatively, 3′ non-translated regulatory sequences can be obtained de novo as, for example, described by An, Methods in Enzymology, 153: 292, 1987, which is incorporated herein by reference.

As the DNA sequence inserted between the transcription initiation site and the start of the coding sequence, i.e., the untranslated 5′ leader sequence (5′UTR), can influence gene expression, one can also employ a particular leader sequence. Suitable leader sequences include those that comprise sequences selected to direct optimum expression of the foreign or endogenous DNA sequence. For example, such leader sequences include a preferred consensus sequence which can increase or maintain mRNA stability and prevent inappropriate initiation of translation as for example described by Joshi, Nucl. Acid Res. 15: 6643, 1987.

Additionally, targeting sequences may be employed to target the enzyme encoded by the foreign or exogenous polynucleotide to an intracellular compartment, for example to the chloroplast, within plant cells or to the extracellular environment. For example, a nucleic acid sequence encoding a transit or signal peptide sequence may be operably linked to a sequence that encodes a chosen enzyme of the subject invention such that, when translated, the transit or signal peptide can transport the enzyme to a particular intracellular or extracellular destination, and can then be optionally post-translationally removed. Transit or signal peptides act by facilitating the transport of proteins through intracellular membranes, e.g., endoplasmic reticulum, vacuole, vesicle, plastid, mitochondrial and plasmalemma membranes. For example, the targeting sequence can direct a desired protein to a particular organelle such as a vacuole or a plastid (e.g., a chloroplast), rather than to the cytosol. Thus, the nucleic acid construct of the invention can further comprise a plastid transit peptide-encoding nucleic acid sequence operably linked between a promoter region and the foreign or exogenous polynucleotide.

### Vectors

The present invention includes use of vectors for manipulation or transfer of genetic constructs. By “vector” is meant a nucleic acid molecule, preferably a DNA molecule derived, for example, from a plasmid, bacteriophage, or plant virus, into which a nucleic acid sequence may be inserted or cloned. A vector preferably contains one or more unique restriction sites and may be capable of autonomous replication in a defined host cell including a target cell or tissue or a progenitor cell or tissue thereof, or be integrable with the genome of the defined host such that the cloned sequence is reproducible. Accordingly, the vector may be an autonomously replicating vector, i.e., a vector that exists as an extrachromosomal entity, the replication of which is independent of chromosomal replication, e.g., a linear or closed circular plasmid, an extrachromosomal element, a minichromosome, or an artificial chromosome. The vector may contain any means for assuring self-replication. Alternatively, the vector may be one which, when introduced into a cell, is integrated into the genome of the recipient cell and replicated together with the chromosome(s) into which it has been integrated. A vector system may comprise a single vector or plasmid, two or more vectors or plasmids, which together contain the total DNA to be introduced into the genome of the host cell, or a transposon. The choice of the vector will typically depend on the compatibility of the vector with the cell into which the vector is to be introduced. The vector may also include a selection marker such as an antibiotic resistance gene, a herbicide resistance gene or other gene that can be used for selection of suitable transformants. Examples of such genes are well known to those of skill in the art.

The nucleic acid construct of the invention can be introduced into a vector, such as a plasmid. Plasmid vectors typically include additional nucleic acid sequences that provide for easy selection, amplification, and transformation of the expression cassette in prokaryotic and eukaryotic cells, e.g., pUC-derived vectors, pSK-derived vectors, pGEM-derived vectors, pSP-derived vectors, or pBS-derived vectors. Additional nucleic acid sequences include origins of replication to provide for autonomous replication of the vector, selectable marker genes, preferably encoding antibiotic or herbicide resistance, unique multiple cloning sites providing for multiple sites to insert nucleic acid sequences or genes encoded in the nucleic acid construct, and sequences that enhance transformation of prokaryotic and eukaryotic (especially plant) cells.

By “marker gene” is meant a gene that imparts a distinct phenotype to cells expressing the marker gene and thus allows such transformed cells to be distinguished from cells that do not have the marker. A selectable marker gene confers a trait for which one can “select” based on resistance to a selective agent (e.g., a herbicide, antibiotic, radiation, heat, or other treatment damaging to untransformed cells). A screenable marker gene (or reporter gene) confers a trait that one can identify through observation or testing, i.e., by “screening” (e.g., β-glucuronidase, luciferase, GFP or other enzyme activity not present in untransformed cells). The marker gene and the nucleotide sequence of interest do not have to be linked.

To facilitate identification of transformants, the nucleic acid construct desirably comprises a selectable or screenable marker gene as, or in addition to, the foreign or exogenous polynucleotide. The actual choice of a marker is not crucial as long as it is functional (i.e., selective) in combination with the plant cells of choice. The marker gene and the foreign or exogenous polynucleotide of interest do not have to be linked, since co-transformation of unlinked genes as, for example, described in U.S. Pat. No. 4,399,216 is also an efficient process in plant transformation.

Examples of bacterial selectable markers are markers that confer antibiotic resistance such as ampicillin, kanamycin, erythromycin, chloramphenicol or tetracycline resistance. Exemplary selectable markers for selection of plant transformants include, but are not limited to, a hyg gene which encodes hygromycin B resistance; a neomycin phosphotransferase (npt) gene conferring resistance to kanamycin, paromomycin, G418 and the like as, for example, described by Potrykus et al., Mol. Gen. Genet. 199: 183, 1985; a glutathione-S-transferase gene from rat liver conferring resistance to glutathione derived herbicides as, for example, described in EP-A 256223; a glutamine synthetase gene conferring, upon overexpression, resistance to glutamine synthetase inhibitors such as phosphinothricin as, for example, described in WO 87/05327, an acetyltransferase gene from Streptomyces viridochromogenes conferring resistance to the selective agent phosphinothricin as, for example, described in EP-A 275957, a gene encoding a 5-enolshikimate-3-phosphate synthase (EPSPS) conferring tolerance to N-phosphonomethylglycine as, for example, described by Hinchee et al., Biotech. 6: 915, 1988, a bar gene conferring resistance against bialaphos as, for example, described in WO 91/02071; a nitrilase gene such as bxn from Klebsiella ozaenae which confers resistance to bromoxynil (Stalker et al., Science, 242: 419, 1988); a dihydrofolate reductase (DHFR) gene conferring resistance to methotrexate (Thillet et al., J. Biol. Chem. 263: 12500, 1988); a mutant acetolactate synthase gene (ALS), which confers resistance to imidazolinone, sulfonylurea or other ALS-inhibiting chemicals (EP-A-154 204); a mutated anthranilate synthase gene that confers resistance to 5-methyl tryptophan; or a dalapon dehalogenase gene that confers resistance to the herbicide.

Preferred screenable markers include, but are not limited to, a uidA gene encoding a β-glucuronidase (GUS) enzyme for which various chromogenic substrates are known, a β-galactosidase gene encoding an enzyme for which chromogenic substrates are known, an aequorin gene (Prasher et al., Biochem. Biophys. Res. Comm. 126: 1259-68, 1985), which may be employed in calcium-sensitive bioluminescence detection; a green fluorescent protein gene (Niedz et al., Plant Cell Reports, 14: 403, 1995); a luciferase (luc) gene (Ow et al., Science, 234: 856, 1986), which allows for bioluminescence detection, and others known in the art. By “reporter molecule” as used in the present specification is meant a molecule that, by its chemical nature, provides an analytically identifiable signal that facilitates determination of promoter activity by reference to protein product.

### Methods of Modifying Gene Expression

The level of a protein, for example an enzyme involved in starch synthesis in developing endosperm of a cereal plant, may be modulated by increasing the level of expression of a nucleotide sequence that codes for the protein in a plant cell, or decreasing the level of expression of a gene encoding the protein in the plant, leading to altered fructan accumulation in grain. The level of expression of a gene may be modulated by altering the copy number per cell, for example by introducing a synthetic genetic construct comprising the coding sequence and a transcriptional control element that is operably connected thereto and that is functional in the cell. A plurality of transformants may be selected and screened for those with a favourable level and/or specificity of transgene expression arising from influences of endogenous sequences in the vicinity of the transgene integration site. A favourable level and pattern of transgene expression is one which results in a substantial increase in fructan levels. This may be detected by simple testing of grain from the transformants. Alternatively, a population of mutagenized grain or a population of plants from a breeding program may be screened for individual lines with altered fructan accumulation.

Reducing gene expression may be achieved through introduction and transcription of a “gene-silencing chimeric gene” introduced into the plant cell. The gene-silencing chimeric gene may be introduced stably into the plant cell's genome, preferably nuclear genome, or it may be introduced transiently, for example on a viral vector. As used herein “gene-silencing effect” refers to the reduction of expression of a target nucleic acid in a a plant cell, which can be achieved by introduction of a silencing RNA. Such reduction may be the result of reduction of transcription, including via methylation of chromatin remodeling, or post-transcriptional modification of the RNA molecules, including via RNA degradation, or both. Gene-silencing includes an abolishing of the expression of the target nucleic acid or gene and a partial effect in either extent or duration. It is sufficient that the level of expression of the target nucleic acid in the presence of the silencing RNA is lower that in the absence thereof. The level of expression may be reduced by at least about 40%, or at least about 50%, or at least about 60%, or at least about 70%, or at least about 80%, or at least about 90%, or at least about 95%, or at least about 99%. The target nucleic acid may be a gene involved in starch synthesis or metabolism, for example starch degradation, but may also include any other endogenous genes, transgenes or exogenous genes such as viral genes which may not be present in the plant cell at the time of introduction of the transgene.

### Antisense RNA Molecules

Antisense techniques may be used to reduce gene expression according to the invention. The term “antisense RNA” shall be taken to mean an RNA molecule that is complementary to at least a portion of a specific mRNA molecule and capable of reducing expression of the gene encoding the mRNA. Such reduction typically occurs in a sequence-dependent manner and is thought to occur by interfering with a post-transcriptional event such as mRNA transport from nucleus to cytoplasm, mRNA stability or inhibition of translation. The use of antisense methods is well known in the art (see for example, Hartmann and Endres, Manual of Antisense Methodology, Kluwer, 1999). The use of antisense techniques in plants has been reviewed by Bourque, Plant Sci. 105: 125-149, 1995 and Senior, Biotech. Genet. Engin. Revs. 15: 79-119, 1998. Bourque, 1995 (supra) lists a large number of examples of how antisense sequences have been utilized in plant systems as a method of gene inactivation. She also states that attaining 100% inhibition of any enzyme activity may not be necessary as partial inhibition will more than likely result in measurable change in the system. Senior, 1998 (supra) states that antisense methods are now a very well established technique for manipulating gene expression.

As used herein, the term “an antisense polynucleotide which hybridises under physiological conditions” means that the polynucleotide (which is fully or partially single stranded) is at least capable of forming a double stranded polynucleotide with an RNA product of the gene to be inhibited, typically the mRNA encoding a protein such as those provided herein, under normal conditions in a cell. Antisense molecules may include sequences that correspond to the structural genes or for sequences that effect control over the gene expression or splicing event. For example, the antisense sequence may correspond to the coding region of the targeted gene, or the 5′-untranslated region (UTR) or the 3′-UTR or combination of these. It may be complementary in part to intron sequences, which may be spliced out during or after transcription, but is preferably complementary only to exon sequences of the target gene. In view of the generally greater divergence of the UTRs, targeting these regions provides greater specificity of gene inhibition.

The length of the antisense sequence should be at least 19 contiguous nucleotides, preferably at least 25 or 30 or 50 nucleotides, and more preferably at least 100, 200, 500 or 1000 nucleotides, to a maximum of the full length of the gene to be inhibited. The full-length sequence complementary to the entire gene transcript may be used. The length is most preferably 100-2000 nucleotides. The degree of identity of the antisense sequence to the targeted transcript should be at least 90% and more preferably 95-100%. The antisense RNA molecule may of course comprise unrelated sequences which may function to stabilize the molecule.

Genetic constructs to express an antisense RNA may be readily made by joining a promoter sequence to a region of the target gene in an “antisense” orientation, which as used herein refers to the reverse orientation relative to the orientation of transcription and translation (if it occurs) of the sequence in the target gene in the plant cell. Accordingly, also provided by this invention is a nucleic acid molecule such as a chimeric DNA coding for an antisense RNA of the invention, including cells, tissues, organs, plants, grain and the like comprising the nucleic acid molecule.

### Ribozymes

The term “ribozyme” as used herein refers to an RNA molecule which specifically recognizes a distinct substrate RNA and catalyzes its cleavage. Typically, the ribozyme contains a region of nucleotides which are complementary to a region of the target RNA, enabling the ribozyme to specifically hybridize to the target RNA under physiological conditions, for example in the cell in which the ribozyme acts, and an enzymatic region referred to herein as the “catalytic domain”. The types of ribozymes that are particularly useful in this invention are the hammerhead ribozyme (Haseloff and Gerlach, Nature 334: 585-591, 1988; Perriman et al., Gene, 113: 157-163, 1992) and the hairpin ribozyme (Shippy et al., Mol. Biotech. 12: 117-129, 1999). DNA encoding the ribozymes can be synthesized using methods well known in the art and may be incorporated into a genetic construct or expression vector for expression in the cell of interest. Accordingly, also provided by this invention is a nucleic acid molecule such as a chimeric DNA coding for a ribozyme of the invention, including cells, tissues, organs, plants, grain and the like comprising the nucleic acid molecule. Typically, the DNA encoding the ribozyme is inserted into an expression cassette under control of a promoter and a transcription termination signal that function in the cell. Specific ribozyme cleavage sites within any potential RNA target may be identified by scanning the target molecule for ribozyme cleavage sites which include the trinucleotide sequences GUA, GUU and GUC. Once identified, short RNA sequences of between about 5 and 20 ribonucleotides corresponding to the region of the target gene 5′ and 3′ of the cleavage site may be evaluated for predicted structural features such as secondary structure that may render the oligonucleotide sequence less suitable. When employed, ribozymes may be selected from the group consisting of hammerhead ribozymes, hairpin ribozymes, axehead ribozymes, newt satellite ribozymes, Tetrahymena ribozymes and RNAse P ribozymes, and are designed according to methods known in the art based on the sequence of the target gene (for instance, see U.S. Pat. No. 5,741,679). The suitability of candidate targets may also be evaluated by testing their accessibility to hybridization with complementary oligonucleotides, using ribonuclease protection assays.

As with antisense polynucleotides described herein, ribozymes of the invention should be capable of hybridizing to a target nucleic acid molecule (for example an mRNA encoding a polypeptide provided as SEQ ID NO:2, SEQ ID NO:4) under “physiological conditions”, namely those conditions within a cell, especially conditions in a plant cell such as a wheat or barley cell.

### RNA Interference/Duplex RNA

As used herein, “artificially introduced dsRNA molecule” refers to the introduction of dsRNA molecule, which may e.g. occur endogenously by transcription from a chimeric gene encoding such dsRNA molecule, however does not refer to the conversion of a single stranded RNA molecule into a dsRNA inside the eukaryotic cell or plant cell. RNA interference (RNAi) is particularly useful for specifically reducing the expression of a gene or inhibiting the production of a particular protein. Although not wishing to be limited by theory, Waterhouse et al., Proc. Natl. Acad. Sci. U.S.A. 95: 13959-13964, 1998 have provided a model for the mechanism by which dsRNA can be used to reduce protein production. This technology relies on the presence of dsRNA molecules that contain a sequence that is essentially identical to the mRNA of the gene of interest or part thereof. Conveniently, the dsRNA can be produced from a single promoter in a recombinant vector or host cell, where the sense and anti-sense sequences are transcribed to produce a hairpin RNA in which the sense and anti-sense sequences hybridize to form the dsRNA region with an intervening sequence or spacer region forming a loop structure, so the hairpin RNA comprises a stem-loop structure. The design and production of suitable dsRNA molecules for the present invention is well within the capacity of a person skilled in the art, particularly considering Waterhouse et al., 1998 (supra), Smith et al., Nature, 407: 319-320, 2000, WO 99/53050, WO 99/49029, and WO 01/34815. Accordingly, also provided by this invention is a nucleic acid molecule such as a chimeric DNA coding for a duplex RNA such as a hairpin RNA of the invention, including cells, tissues, organs, plants, grain and the like comprising the nucleic acid molecule.

In one example, a DNA is introduced that directs the synthesis of an at least partly double stranded RNA product(s) with homology to the target gene to be inactivated. The DNA therefore comprises both sense and antisense sequences that, when transcribed into RNA, can hybridize to form the double-stranded RNA region. In a preferred embodiment, the sense and antisense sequences are separated by a spacer region that comprises an intron which, when transcribed into RNA, is spliced out. This arrangement has been shown to result in a higher efficiency of gene silencing (Smith et al., 2000 (supra)). The double-stranded region may comprise one or two RNA molecules, transcribed from either one DNA region or two. The dsRNA may be classified as long hpRNA, having long, sense and antisense regions which can be largely complementary, but need not be entirely complementary (typically forming a basepaired region larger than about 100 bp, preferably ranging between 200-1000 bp). hpRNA can also be smaller with the double-stranded portion ranging in size from about 30 to about 50 bp, or from 30 to about 100 bp (see WO 04/073390, herein incorporated by reference). The presence of the double stranded RNA region is thought to trigger a response from an endogenous plant system that processes the double stranded RNA to oligonucleotides of 21-24 nucleotides long, and also uses these oligonucleotides for sequence-specific cleavage of the homologous RNA transcript from the target plant gene, efficiently reducing or eliminating the activity of the target gene.

The length of the sense and antisense sequences that hybridise should each be at least 19 contiguous nucleotides, preferably at least 27 or 30 or 50 nucleotides, and more preferably at least 100, 200, or 500 nucleotides, up to the full-length sequence corresponding to the entire gene transcript. The lengths are most preferably 100-2000 nucleotides. The degree of identity of the sense and antisense sequences to the targeted transcript should be at least 85%, preferably at least 90% and more preferably 95-100%. The longer the sequence, the less stringent the requirement is for overall sequence identity. The RNA molecule may of course comprise unrelated sequences which may function to stabilize the molecule. The RNA molecule may be a hybrid between different sequences targeting different target RNAs, allowing reduction in expression of more than one target gene, or it may be one sequence which corresponds to a family of related target genes such as a multigene family. The sequences used in the dsRNA preferably correspond to exon sequences of the target gene and may correspond to 5′ and/or 3′ untranslated sequences or protein coding sequences or any combination thereof.

The promoter used to express the dsRNA-forming construct may be any type of promoter if the resulting dsRNA is specific for a gene product in the cell lineage targeted for destruction. Alternatively, the promoter may be lineage specific in that it is only expressed in cells of a particular development lineage. This might be advantageous where some overlap in homology is observed with a gene that is expressed in a non-targeted cell lineage. The promoter may also be inducible by externally controlled factors, or by intracellular environmental factors. Typically, the RNA molecule is expressed under the control of a RNA polymerase II or RNA polymerase III promoter. Examples of the latter include tRNA or snRNA promoters.

Other silencing RNA may be “unpolyadenylated RNA” comprising at least 20 consecutive nucleotides having at least 95% sequence identity to the complement of a nucleotide sequence of an RNA transcript of the target gene, such as described in WO 01/12824 or U.S. Pat. No. 6,423,885 (both documents herein incorporated by reference). Yet another type of silencing RNA is an RNA molecule as described in WO 03/076619 (herein incorporated by reference) comprising at least 20 consecutive nucleotides having at least 95% sequence identity to the sequence of the target nucleic acid or the complement thereof, and further comprising a largely-double stranded region as described in WO 03/076619.

MicroRNA regulation is a specialized branch of the RNA silencing pathway that evolved towards gene regulation, diverging from conventional RNAi/PTGS. MicroRNAs are a specific class of small RNAs that are encoded in gene-like elements organized in a characteristic partial inverted repeat. When transcribed, microRNA genes give rise to partially basepaired stem-looped precursor RNAs from which the microRNAs are subsequently processed. MicroRNAs are typically about 21 nucleotides in length. The released miRNAs are incorporated into RISC-like complexes containing a particular subset of Argonaute proteins that exert sequence-specific gene repression (see, for example, Millar and Waterhouse, Funct Integr Genomics, 5: 129-135, 2005; Pasquinelli et al., Curr Opin Genet Develop 15: 200-205, 2005; Almeida and Allshire, Trends Cell Biol. 15: 251-258, 2005, herein incorporated by reference).

### Cosuppression

Another molecular biological approach that may be used for specifically reducing gene expression is co-suppression. The mechanism of co-suppression is not well understood but is thought to involve post-transcriptional gene silencing (PTGS) and in that regard may be very similar to many examples of antisense suppression. It involves introducing an extra copy of a gene or a fragment thereof into a plant in the “sense orientation” with respect to a promoter for its expression, which as used herein refers to the same orientation as transcription and translation (if it occurs) of the sequence relative to the sequence in the target gene. The size of the sense fragment, its correspondence to target gene regions, and its degree of homology to the target gene are as for the antisense sequences described above. In some instances the additional copy of the gene sequence interferes with the expression of the target plant gene. Reference is made to Patent specification WO 97/20936 and European patent specification 0465572 for methods of implementing co-suppression approaches. The antisense, co-suppression or double stranded RNA molecules may also comprise a largely double-stranded RNA region, preferably comprising a nuclear localization signal, as described in WO 03/076619.

Any of these technologies for reducing gene expression can be used to coordinately reduce the activity of multiple genes. For example, one RNA molecule can be targeted against a family of related genes by targeting a region of the genes which is in common. Alternatively, unrelated genes may be targeted by including multiple regions in one RNA molecule, each region targeting a different gene. This can readily be done by fusing the multiple regions under the control of a single promoter.

Methods of Introducing Nucleic Acids into Plant Cells/Transformation

A number of techniques are available for the introduction of nucleic acid molecules into a plant host cell, well known to workers in the art. The term “transformation” means alteration of the genotype of an organism, for example a bacterium or a plant, by the introduction of a foreign or exogenous nucleic acid. By “transformant” is meant an organism so altered. As used herein the term “transgenic” refers to a genetically modified plant in which the endogenous genome is supplemented or modified by the integration, or stable maintenance in a replicable non-integrated form, of an introduced foreign or exogenous gene or sequence. By “transgene” is meant a foreign or exogenous gene or sequence that is introduced into the genome of a plant. The nucleic acid molecule may be stably integrated into the genome of the plant, or it may be replicated as an extrachromosomal element. By “genome” is meant the total inherited genetic complement of the cell, plant or plant part, and includes chromosomal DNA, plastid DNA, mitochondrial DNA and extrachromosomal DNA molecules. The term “regeneration” as used herein in relation to plant materials means growing a whole, differentiated plant from a plant cell, a group of plant cells, a plant part such as, for example, from an embryo, scutellum, protoplast, callus, or other tissue, but not including growth of a plant from a seed.

The particular choice of a transformation technology will be determined by its efficiency to transform certain plant species as well as the experience and preference of the person practicing the invention with a particular methodology of choice. It will be apparent to the skilled person that the particular choice of a transformation system to introduce a nucleic acid construct into plant cells is not essential to or a limitation of the invention, provided it achieves an acceptable level of nucleic acid transfer. Guidance in the practical implementation of transformation systems for plant improvement is provided by Birch, Ann Rev Plant Physiol Plant Mol Biol. 48: 297-326, 1997.

In principle, both dicotyledonous and monocotyledonous plants that are amenable to transformation can be modified by introducing a nucleic acid construct according to the invention into a recipient cell and growing a new plant that harbors and expresses a polynucleotide according to the invention.

Introduction and expression of foreign or exogenous polynucleotides in dicotyledonous plants such as tobacco, potato and legumes or monocotyledonous plants such as cereals, including wheat, barley, rice, corn, oats, rye and sorghum has been shown to be possible using the T-DNA of the tumor-inducing (Ti) plasmid of Agrobacterium tumefaciens (See, for example, Umbeck, U.S. Pat. No. 5,004,863, and International application PCT/US93/02480). A construct of the invention may be introduced into a plant cell utilizing A. tumefaciens containing the Ti plasmid. In using an A. tumefaciens culture as a transformation vehicle, it is most advantageous to use a non-oncogenic strain of the Agrobacterium as the vector carrier so that normal non-oncogenic differentiation of the transformed tissues is possible. It is preferred that the Agrobacterium harbors a binary Ti plasmid system. Such a binary system comprises (1) a first Ti plasmid having a virulence region essential for the introduction of transfer DNA (T-DNA) into plants, and (2) a chimeric plasmid. The chimeric plasmid contains at least one border region of the T-DNA region of a wild-type Ti plasmid flanking the nucleic acid to be transferred. Binary Ti plasmid systems have been shown effective to transform plant cells as, for example, described by De Framond, Biotechnology, 1: 262, 1983 and Hoekema et al., Nature, 303: 179, 1983. Such a binary system is preferred inter alia because it does not require integration into the Ti plasmid in Agrobacterium.

Methods involving the use of Agrobacterium include, but are not limited to: (a) co-cultivation of Agrobacterium with cultured isolated protoplasts; (b) transformation of plant cells or tissues with Agrobacterium; (c) transformation of seeds, apices or meristems with Agrobacterium, or (d) inoculation in planta such as the floral-dip method as described by Bechtold et al., C.R. Acad. Sci. Paris, 316: 1194, 1993 or in wheat (as described in WO 00/63398, herein incorporated by reference). This approach is based on the infiltration of a suspension of Agrobacterium cells. Alternatively, the chimeric construct may be introduced using root-inducing (Ri) plasmids of Agrobacterium as vectors.

Methods for transformation of cereal plants such as wheat and barley for introducing genetic variation into the plant by introduction of an exogenous nucleic acid and for regeneration of plants from protoplasts or immature plant embryos are well known in the art, see for example, Wan and Lemaux, Plant Physiol. 104: 37-48, 1994, Tingay et al., Plant J. 11: 1369-1376, 1997, Canadian Patent Application No. 2,092,588, Australian Patent Application No. 61781/94, Australian Patent No. 667939, U.S. Pat. No. 6,100,447, International Patent Application PCT/US97/10621, U.S. Pat. No. 5,589,617, U.S. Pat. No. 6,541,257. Preferably, transgenic wheat, barley or other cereal plants are produced by Agrobacterium tumefaciens mediated transformation procedures. Vectors carrying the desired nucleic acid construct may be introduced into regenerable cereal cells of tissue cultured plants or explants. The regenerable cells are preferably from the scutellum of immature embryos, mature embryos, callus derived from these, or the meristematic tissue. Immature embryos are preferably those from inflorescences about 10-15 days after anthesis.

The genetic construct can also be introduced into plant cells by electroporation as, for example, described by Fromm et al., Proc. Natl. Acad. Sci. U.S.A. 82: 5824, 1985 and Shimamoto et al., Nature, 338: 274-276, 1989. In this technique, plant protoplasts are electroporated in the presence of vectors or nucleic acids containing the relevant nucleic acid sequences. Electrical impulses of high field strength reversibly permeabilize membranes allowing the introduction of nucleic acids. Electroporated plant protoplasts reform the cell wall, divide and form a plant callus.

Another method for introducing the nucleic acid construct into a plant cell is high velocity ballistic penetration by small particles (also known as particle bombardment or microprojectile bombardment) with the nucleic acid to be introduced contained either within the matrix of small beads or particles, or on the surface thereof as, for example described by Klein et al., Nature, 327: 70, 1987.

Alternatively, the nucleic acid construct can be introduced into a plant cell by contacting the plant cell using mechanical or chemical means. For example, a nucleic acid can be mechanically transferred by microinjection directly into plant cells by use of micropipettes. Alternatively, a nucleic acid may be transferred into the plant cell by using polyethylene glycol which forms a precipitation complex with genetic material that is taken up by the cell.

### Mutagenesis

The plants of the invention can be produced and identified after mutagenesis. This may provide a plant which is non-transgenic, which is desirable in some markets.

Mutants can be either naturally occurring (that is to say, isolated from a natural source) or synthetic (for example, by performing mutagenesis on the nucleic acid) or induced. Generally, a progenitor plant cell, tissue, seed or plant may be subjected to mutagenesis to produce single or multiple mutations, such as nucleotide substitutions, deletions, additions and/or codon modification. In the context of this application, an “induced mutation” is an artificially induced genetic variation which may be the result of chemical, radiation or biologically-based mutagenesis, for example transposon or T-DNA insertion. Preferred mutations are null mutations such as nonsense mutations, frameshift mutations, insertional mutations or splice-site variants which completely inactivate the gene. Nucleotide insertional derivatives include 5′ and 3′ terminal fusions as well as intra-sequence insertions of single or multiple nucleotides. Insertional nucleotide sequence variants are those in which one or more nucleotides are introduced into the nucleotide sequence, which may be obtained by random insertion with suitable screening of the resulting products. Deletional variants are characterised by the removal of one or more nucleotides from the sequence. Preferably, a mutant gene has only a single insertion or deletion of a sequence of nucleotides relative to the wild-type gene. Substitutional nucleotide variants are those in which at least one nucleotide in the sequence has been removed and a different nucleotide inserted in its place. The preferred number of nucleotides affected by substitutions in a mutant gene relative to the wild-type gene is a maximum of ten nucleotides, more preferably a maximum of 9, 8, 7, 6, 5, 4, 3, or 2, or only one nucleotide. Such a substitution may be “silent” in that the substitution does not change the amino acid defined by the codon. Alternatively, conservative substituents are designed to alter one amino acid for another similar acting amino acid. Typical conservative substitutions are those made in accordance with Table 9 “Exemplary substitutions”.

The term “mutation” as used herein does not include silent nucleotide substitutions which do not affect the activity of the gene, and therefore includes only alterations in the gene sequence which affect the gene activity. The term “polymorphism” refers to any change in the nucleotide sequence including such silent nucleotide substitutions.

In a preferred embodiment, the plant comprises a deletion of at least part of a SSII gene or a frameshift or splice site variation in such gene.

Mutagenesis can be achieved by chemical or radiation means, for example EMS or sodium azide (Zwar and Chandler, Planta 197: 39-48, 1995) treatment of seed, or gamma irradiation, well know in the art. Isolation of mutants may be achieved by screening mutagenised plants or seed. For example, a mutagenized population of cereal plants may be screened for low SSII activity in the leaf or grain starch, mutation of the SSII gene by a PCR or heteroduplex based assay, or loss of the SSII protein by ELISA. In a polyploid plant, screening is preferably done in a genotype that already lacks one or two of the SSII activities, for example in a wheat plant already mutant in the SSII genes on two of the three genomes, so that a mutant entirely lacking the functional activity is sought. Alternatively, the mutation may be identified using techniques such as “tilling” in a population mutagenised with an agent such as EMS (Slade and Knauf, Transgenic Res. 14: 109-115, 2005). Such mutations may then be introduced into desirable genetic backgrounds by crossing the mutant with a plant of the desired genetic background and performing a suitable number of backcrosses to cross out the originally undesired parent background.

The mutation may have been introduced into the plant directly by mutagenesis or indirectly by crossing of two parental plants, one of which comprised the introduced mutation. The modified plants such as cereal plants may be transgenic or non-transgenic. Using mutagenesis, a non-transgenic plant lacking the function of interest may be produced. The invention also extends to the grain or other plant parts produced from the plants and any propagating material of the plants that can be used to produce the plants with the desired characteristics, such as cultured tissue or cells. The invention clearly extends to methods of producing or identifying such plants or the grain produced by such plants.

Plants of the invention can be produced using the process known as TILLING (Targeting Induced Local Lesions IN Genomes). In a first step, introduced mutations such as novel single base pair changes are induced in a population of plants by treating cells, seeds, pollen or other plant parts with a chemical mutagen or radiation, and then advancing plants to a generation where mutations will be stably inherited. DNA is extracted, and seeds are stored from all members of the population to create a resource that can be accessed repeatedly over time.

For a TILLING assay, PCR primers are designed to specifically amplify a single gene target of interest. Specificity is especially important if a target is a member of a gene family or part of a polyploid genome. Next, dye-labeled primers can be used to amplify PCR products from pooled DNA of multiple individuals. These PCR products are denatured and reannealed to allow the formation of mismatched base pairs. Mismatches, or heteroduplexes, represent both naturally occurring single nucleotide polymorphisms (SNPs) (i.e., several plants from the population are likely to carry the same polymorphism) and induced SNPs (i.e., only rare individual plants are likely to display the mutation). After heteroduplex formation, the use of an endonuclease, such as Cell, that recognizes and cleaves mismatched DNA is the key to discovering novel SNPs within a TILLING population.

Using this approach, many thousands of plants can be screened to identify any individual with a single base change as well as small insertions or deletions (1-30 bp) in any gene or specific region of the genome. Genomic fragments being assayed can range in size anywhere from 0.3 to 1.6 kb. At 8-fold pooling, 1.4 kb fragments (discounting the ends of fragments where SNP detection is problematic due to noise) and 96 lanes per assay, this combination allows up to a million base pairs of genomic DNA to be screened per single assay, making TILLING a high-throughput technique. TILLING is further described in Slade and Knauf, 2005 (supra), and Henikoff et al., Plant Physiol. 135: 630-636, 2004, herein incorporated by reference.

In addition to allowing efficient detection of mutations, high-throughput TILLING technology is ideal for the detection of natural polymorphisms. Therefore, interrogating an unknown homologous DNA by heteroduplexing to a known sequence reveals the number and position of polymorphic sites. Both nucleotide changes and small insertions and deletions are identified, including at least some repeat number polymorphisms. This has been called Ecotilling (Comai et al., Plant J. 37: 778-786, 2004).

Each SNP is recorded by its approximate position within a few nucleotides. Thus, each haplotype can be archived based on its mobility. Sequence data can be obtained with a relatively small incremental effort using aliquots of the same amplified DNA that is used for the mismatch-cleavage assay. The left or right sequencing primer for a single reaction is chosen by its proximity to the polymorphism. Sequencher software performs a multiple alignment and discovers the base change, which in each case confirmed the gel band.

Ecotilling can be performed more cheaply than full sequencing, the method currently used for most SNP discovery. Plates containing arrayed ecotypic DNA can be screened rather than pools of DNA from mutagenized plants. Because detection is on gels with nearly base pair resolution and background patterns are uniform across lanes, bands that are of identical size can be matched, thus discovering and genotyping SNPs in a single step. In this way, ultimate sequencing of the SNP is simple and efficient, made more so by the fact that the aliquots of the same PCR products used for screening can be subjected to DNA sequencing.

### Genetic Linkage

As used herein, the term “genetically linked” refers to a marker locus and a second locus being sufficiently close on a chromosome that they will be inherited together in more than 50% of meioses, e.g., not randomly. This definition includes the situation where the marker locus and second locus form part of the same gene. Furthermore, this definition includes the situation where the marker locus comprises a polymorphism that is responsible for the trait of interest (in other words the marker locus is directly “linked” to the phenotype). Thus, the percent of recombination observed between the loci per generation (centimorgans (cM)), will be less than 50. In particular embodiments of the invention, genetically linked loci may be 45, 35, 25, 15, 10, 5, 4, 3, 2, or 1 or less cM apart on a chromosome. Preferably, the markers are less than 5 cM or 2 cM apart and most preferably about 0 cM apart.

As used herein, the “other genetic markers” may be any molecules which are linked to a desired trait of a cereal plant such as wheat or barley. Such markers are well known to those skilled in the art and include molecular markers linked to genes determining traits such disease resistance, yield, plant morphology, grain quality, other dormancy traits such as grain colour, gibberellic acid content in the seed, plant height, flour colour and the like. Examples of such genes in wheat are stem-rust resistance genes Sr2 or Sr38, the stripe rust resistance genes Yr10 or Yr17, the nematode resistance genes such as Cre1 and Cre3, alleles at glutenin loci that determine dough strength such as Ax, Bx, Dx, Ay, By and Dy alleles, the Rht genes that determine a semi-dwarf growth habit and therefore lodging resistance (Eagles et al., Aust. J. Agric. Res. 52: 1 349-1356, 2001; Langridge et al., Aust. J. Agric. Res. 52: 1043-1077, 2001; Sharp et al., Aust JAgric Res 52: 1357-1366, 2001).

Marker assisted selection is a well recognised method of selecting for heterozygous plants required when backcrossing with a recurrent parent in a classical breeding program. The population of plants in each backcross generation will be heterozygous for the gene of interest normally present in a 1:1 ratio in a backcross population, and the molecular marker can be used to distinguish the two alleles of the gene. By extracting DNA from, for example, young shoots and testing with a specific marker for the introgressed desirable trait, early selection of plants for further backcrossing is made whilst energy and resources are concentrated on fewer plants.

Any molecular biological technique known in the art which is capable of detecting alleles of an SSII or other gene can be used in the methods of the present invention. Such methods include, but are not limited to, the use of nucleic acid amplification, nucleic acid sequencing, nucleic acid hybridization with suitably labeled probes, single-strand conformational analysis (SSCA), denaturing gradient gel electrophoresis (DGGE), heteroduplex analysis (HET), chemical cleavage analysis (CCM), catalytic nucleic acid cleavage or a combination thereof (see, for example, Lemieux, Current Genomics, 1: 301-311, 2000; Langridge et al., 2001 (supra)). The invention also includes the use of molecular marker techniques to detect polymorphisms linked to alleles of (for example) an SSII gene which confers altered fructan accumulation. Such methods include the detection or analysis of restriction fragment length polymorphisms (RFLP), RAPD, amplified fragment length polymorphisms (AFLP) and microsatellite (simple sequence repeat, SSR) polymorphisms. The closely linked markers can be obtained readily by methods well known in the art, such as Bulked Segregant Analysis, as reviewed by Langridge et al., 2001 (supra).

The “polymerase chain reaction” (“PCR”) is a reaction in which replicate copies are made of a target polynucleotide using a “pair of primers” or “set of primers” consisting of “upstream” and a “downstream” primer, and a catalyst of polymerization, such as a DNA polymerase, and typically a thermally-stable polymerase enzyme. Methods for PCR are known in the art, and are taught, for example, in “PCR” (McPherson and Moller (Ed), BIOS Scientific Publishers Ltd, Oxford, 2000). PCR can be performed on cDNA obtained from reverse transcribing mRNA isolated from plant cells expressing an SSII gene or on genomic DNA isolated from a plant.

A primer is an oligonucleotide sequence that is capable of hybridising in a sequence specific fashion to the target sequence and being extended during the PCR. Amplicons or PCR products or PCR fragments or amplification products are extension products that comprise the primer and the newly synthesized copies of the target sequences. Multiplex PCR systems contain multiple sets of primers that result in simultaneous production of more than one amplicon. Primers may be perfectly matched to the target sequence or they may contain internal mismatched bases that can result in the introduction of restriction enzyme or catalytic nucleic acid recognition/cleavage sites in specific target sequences. Primers may also contain additional sequences and/or contain modified or labelled nucleotides to facilitate capture or detection of amplicons. Repeated cycles of heat denaturation of the DNA, annealing of primers to their complementary sequences and extension of the annealed primers with polymerase result in exponential amplification of the target sequence. The terms target or target sequence or template refer to nucleic acid sequences which are amplified.

Methods for direct sequencing of nucleotide sequences are well known to those skilled in the art and can be found for example in Ausubel et al. (supra) and Sambrook et al. (supra). Sequencing can be carried out by any suitable method, for example, dideoxy sequencing, chemical sequencing or variations thereof. Direct sequencing has the advantage of determining variation in any base pair of a particular sequence.

### Plants

The term “plant” as used herein as a noun refers to whole plants and refers to any member of the Kingdom Plantae, but as used as an adjective refers to any substance which is present in, obtained from, derived from, or related to a plant, such as for example, plant organs (e.g. leaves, stems, roots, flowers), single cells (e.g. pollen), seeds, plant cells and the like. Plantlets and germinated seeds from which roots and shoots have emerged are also included within the meaning of “plant”. The term “plant parts” as used herein refers to one or more plant tissues or organs which are obtained from a plant and which comprises genomic DNA of the plant. Plant parts include vegetative structures (for example, leaves, stems), roots, floral organs/structures, seed (including embryo, endosperm, and seed coat), plant tissue (for example, vascular tissue, ground tissue, and the like), cells and progeny of the same. The term “plant cell” as used herein refers to a cell obtained from a plant or in a plant and includes protoplasts or other cells derived from plants, gamete-producing cells, and cells which regenerate into whole plants. Plant cells may be cells in culture. By “plant tissue” is meant differentiated tissue in a plant or obtained from a plant (“explant”) or undifferentiated tissue derived from immature or mature embryos, seeds, roots, shoots, fruits, tubers, pollen, tumor tissue, such as crown galls, and various forms of aggregations of plant cells in culture, such as calli. Exemplary plant tissues in or from seeds are endosperm, scutellum, aleurone layer and embryo. The invention accordingly includes plants and plant parts and products comprising these, particularly grain comprising fructan.

As used herein, the term “grain” refers to mature seed of a plant, such as is typically harvested commercially in the field. Mature cereal grain such as wheat or barley commonly has a moisture content of less than about 18-20%.

A “transgenic plant” as used herein refers to a plant that contains a gene construct not found in a wild-type plant of the same species, variety or cultivar. That is, transgenic plants (transformed plants) contain genetic material (a transgene) that they did not contain prior to the transformation. The transgene may include genetic sequences obtained from or derived from a plant cell, or another plant cell, or a non-plant source, or a synthetic sequence. Typically, the transgene has been introduced into the plant by human manipulation such as, for example, by transformation but any method can be used as one of skill in the art recognizes. The genetic material is preferably stably integrated into the genome of the plant. The introduced genetic material may comprise sequences that naturally occur in the same species but in a rearranged order or in a different arrangement of elements, for example an antisense sequence. Plants containing such sequences are included herein in “transgenic plants”. A “non-transgenic plant” is one which has not been genetically modified by the introduction of genetic material by recombinant DNA techniques. In a preferred embodiment, the transgenic plants are homozygous for each and every gene that has been introduced (transgene) so that their progeny do not segregate for the desired phenotype.

As used herein, the term “corresponding non-transgenic plant” refers to a plant which is isogenic relative to the transgenic plant but without the transgene of interest. Preferably, the corresponding non-transgenic plant is of the same cultivar or variety as the progenitor of the transgenic plant of interest, or a sibling plant line which lacks the construct, often termed a “segregant”, or a plant of the same cultivar or variety transformed with an “empty vector” construct, and may be a non-transgenic plant. “Wild type”, as used herein, refers to a cell, tissue or plant that has not been modified according to the invention. Wild-type cells, tissue or plants may be used as controls to compare levels of expression of an exogenous nucleic acid or the extent and nature of trait modification with cells, tissue or plants modified as described herein.

Transgenic plants, as defined in the context of the present invention include progeny of the plants which have been genetically modified using recombinant techniques, wherein the progeny comprise the transgene of interest. Such progeny may be obtained by self-fertilisation of the primary transgenic plant or by crossing such plants with another plant of the same species. This would generally be to modulate the production of at least one protein/enzyme defined herein in the desired plant or plant organ. Transgenic plant parts include all parts and cells of said plants comprising the transgene such as, for example, cultured tissues, callus and protoplasts.

Any of several methods may be employed to determine the presence of a transgene in a transformed plant. For example, polymerase chain reaction (PCR) may be used to amplify sequences that are unique to the transformed plant, with detection of the amplified products by gel electrophoresis or other methods. DNA may be extracted from the plants using conventional methods and the PCR reaction carried out using primers to amplify a specific DNA, the presence of which will distinguish the transformed and non-transformed plants. For example, primers may be designed that will amplify a region of DNA from the transformation vector reading into the construct and the reverse primer designed from the gene of interest. These primers will only amplify a fragment if the plant has been successfully transformed. An alternative method to confirm a positive transformant is by Southern blot hybridization, well known in the art. Plants which are transformed may also be identified i.e. distinguished from non-transformed or wild-type plants by their phenotype, for example conferred by the presence of a selectable marker gene, or conferred by the phenotype of altered fructan content of the grain of the plant, or related phenotype such as altered starch synthase activity.

As used herein, “germination” refers to the emergence of the root tip from the seed coat after imbibition. “Germination rate” refers to the percentage of seeds in a population which have germinated over a period of time, for example 7 or 10 days, after imbibition. A population of seeds can be assessed daily over several days to determine the germination percentage over time. With regard to seeds of the present invention, as used herein the term “germination rate which is substantially the same” means that the germination rate of the transgenic seeds is at least 90%, that of isogenic wild-type seeds.

Plants provided by or contemplated for use in the practice of the present invention include angiosperms, including both monocotyledons and dicotyledons. In preferred embodiments, the plants of the present invention are crop plants (for example, cereals and pulses, maize, wheat, potatoes, tapioca, rice, sorghum, millet, cassava, barley, or pea), or other legumes. The plants may be grown for production of edible roots, tubers, leaves, stems, flowers or fruit. Preferably, the plant is a cereal plant. Examples of cereal plants include, but are not limited to, wheat, barley, rice, maize (corn), sorghum, oats, and rye. In one embodiment, the cereal plant is other than barley mutants M292, M342 or barley plants comprising the same SSII mutation, described in WO 02/37955, herein incorporated by reference, such as wheat, rice, maize or sorghum.

As used herein, the term “wheat” refers to any species of the Genus Triticum, including progenitors thereof, as well as progeny thereof produced by crosses with other species. As is understood in the art, hexaploid wheats such as bread wheat comprise three genomes which are commonly designated the A, B and D genomes, while tetraploid wheats such as durum wheat comprise two genomes commonly designated the A and B genomes. Each wheat genome comprises 7 pairs of chromosomes which may be observed by cytological methods during meiosis and thus identified, as is well known in the art. Wheat includes “hexaploid wheat” which has genome organization of AABBDD, comprised of 42 chromosomes, and “tetraploid wheat” which has genome organization of AABB, comprised of 28 chromosomes. Hexaploid wheat includes T. aestivum, T. spelta, T. macha, T. compactum, T. sphaerococcum, T. vavilovii, and interspecies cross thereof. Tetraploid wheat includes T. durum (also referred to herein as durum wheat or Triticum turgidum ssp. durum), T. dicoccoides, T. dicoccum, T. polonicum, and interspecies cross thereof. In addition, the term “wheat” includes potential progenitors of hexaploid or tetraploid Triticum sp. such as T. uartu, T. monococcum or T. boeoticum for the A genome, Aegilops speltoides for the B genome, and T. tauschii (also known as Aegilops squarrosa or Aegilops tauschii) for the D genome. A wheat cultivar for use in the present invention may belong to, but is not limited to, any of the above-listed species. Also encompassed are plants that are produced by conventional techniques using Triticum sp. as a parent in a sexual cross with a non-Triticum species (such as rye [Secale cereale]), including but not limited to Triticale. Preferably, the wheat plant is suitable for commercial production of grain, such as commercial varieties of hexaploid wheat or durum wheat, having suitable agronomic characteristics which are known to those skilled in the art.

As used herein, the term “barley” refers to any species of the Genus Hordeum, including progenitors thereof, as well as progeny thereof produced by crosses with other species. It is preferred that the plant is of a Hordeum species which is commercially cultivated such as, for example, a strain or cultivar or variety of Hordeum vulgare or suitable for commercial production of grain.

### Food Production

In another aspect, the invention provides cereal plants and grain, and products obtained therefrom comprising fructan from the grain, that is useful for food or feed production, the grain having increased levels of fructan compared to corresponding wild-type grain. Preferably the plant from which the grain is obtained has a reduced level of SSII activity in the endosperm during development. The plant of the present invention is useful for food production and in particular for commercial food production. Such food production might include the making of flour, dough or other products that might be an ingredient in commercial food production. In an embodiment which is desirable for use in food production, the seed or grain of the plant has a fructan content that is increased relative to the wild-type plant. The grain may have a level of activity of degradative enzymes, particularly of one or more amylases such as α-amylase or β-amylase, which is reduced by the presence of a transgene or an introduced mutation which reduces expression of a gene encoding such a degradative enzyme in the grain. Flour or dough from such grain has desirable properties for baking or other food production.

The desired genetic background of the plant will include considerations of agronomic yield and other characteristics. Such characteristics might include whether it is desired to have a winter or spring types, agronomic performance, disease resistance and abiotic stress resistance. For Australian use, one might want to cross the altered fructan trait into wheat cultivars such as Baxter, Kennedy, Janz, Frame, Rosella, Cadoux, Diamondbird or other commonly grown varieties. Other varieties will be suited for other growing regions. It is preferred that the plant, preferably wheat, variety of the invention provide a yield not less than 80% of the corresponding wild-type variety in at least some growing regions, more preferably not less than 85% and even more preferably not less than 90%. The yield can readily be measured in controlled field trials.

In further embodiments, other desirable characteristics include the capacity to mill the grain, in particular the grain hardness. Another aspect that might make a plant of higher value is the degree of fructan or starch extraction from the grain, the higher extraction rates being more useful, or the protein content, the ration of amylose to amylopectin, or the content of other non-starch polysaccharides such as β-glucan which also contribute to the dietary fibre content of the grain products. Grain shape is also another feature that can impact on the commercial usefulness of a plant, thus grain shape can have an impact on the ease or otherwise with which the grain can be milled.

Starch is readily isolated from grain of the invention using standard methods, for example the method of Schulman and Kammiovirta, Starch, 43: 387-389, 1991. On an industrial scale, wet or dry milling can be used. Starch granule size is important in the starch processing industry where there is separation of the larger A granules from the smaller B granules.

### Food Products

The invention also encompasses foods, beverages or pharmaceutical preparations produced with products, preferably those comprising increased fructan, obtained from the plants or grain of the invention. Such food production might include the making of processed grain, wholemeal, flour, dough or other products that might be an ingredient in commercial food production. The grain of the invention or products derived therefrom containing fructan may be used in a variety of food applications for human consumption. As used herein, “humans” refers to Homo sapiens. The grain can be used readily in food processing procedures and therefore the invention includes milled, ground, kibbled, pearled or rolled grain or products obtained from the processed or whole grain of the plants of the invention, including flour. These products may be then used in various food products, for example farinaceous products such as breads, cakes, biscuits and the like or food additives such as thickeners or binding agents or to make drinks, noodles, pasta or quick soups. The grain or products derived from the grain of the invention are particularly desired in breakfast cereals or as extruded products. The fructan may be incorporated into fat or oil products such as margarine or shortening, salad dressing, egg products such as mayonnaise, dairy products such as icecream, yogurt or cheese, cereal products such as corn or wheat flour, fruit juices, other foods or food materials, or the fructan may be processed into beverages or foods such as bread, cake, biscuits, breakfast cereals, pasta, noodles or sauces. Fructan is also useful as a low calorie sweetening product.

In bread, the ingredients comprising fructan which may be in the form of flour or wholemeal may substitute for 10% (w/w) or more of unaltered flour or wholemeal, preferably substituting at least 30% and even more preferably at least 50% of the unaltered flour or wholemeal. The formulation might therefore be, for example, flour 70 parts, high-fructan starch 30 parts, fat 2 parts, salt 2 parts, improver 1 part, yeast 2.5 parts. The production of the bread may be by a rapid dough technique or other techniques as is known by those skilled in the art.

Alternatively, the high-fructan product of the invention may be incorporated into a farinaceous based pasta product. The amount of fructan of the invention employed in the pasta composition may be in the range of 5-20% (w/w) based on the total weight of farinaceous material more particularly in the range of 10 to 20%. Suitable other farinaceous materials will readily be chosen by a person skilled in the art. Other material may also be added to the composition for example dry or liquid eggs (yolks, whites, or both) or high protein substances such as milk protein or fish protein. Vitamins, minerals, calcium salts, amino acids, buffering agents such as disodium hydrogen phosphate, seasoning, gum, gluten or glyceryl monostearate may also be added.

Other parts of the plants of the invention that are edible may be used as foods for human consumption or as feed for animal use. For example, leaves, stems, roots, tubers, fruit, pods or extracts or parts of these comprising cells of the invention from any of these may be used for human or animal consumption. Increased fructan content of the plants of the invention and parts thereof may provide advantages for use of these materials as animal feed such as, for example, as feed for pigs, cattle, horses, poultry such as chickens and other animals.

### Methods

The products or compounds of the present invention can be formulated in pharmaceutic compositions which are prepared according to conventional pharmaceutical compounding techniques. See, for example, Remington's Pharmaceutical Sciences, 18^{th} Ed. Mack Publishing, Company, Easton, PA, U.S.A., 1990. The composition may contain the active agent or pharmaceutically acceptable derivative active agent. These compositions may comprise, in addition to one of the active substances, a pharmaceutically acceptable excipient, carrier, buffer, stabilizer or other materials well known in the art. Such materials should be non-toxic and should not interfere with the efficacy of the active ingredient. The carrier may take a wide variety of forms depending on the form of preparation desired for administration.

For oral administration, the compounds can be formulated into solid or liquid preparations such as capsules, pills, tablets, lozenges, powders, suspensions or emulsions. In preparing the compositions in oral dosage form, any of the usual pharmaceutical media may be employed, such as, for example, water, glycols, oils, alcohols, flavoring agents, preservatives, coloring agents, suspending agents, and the like in the case of oral liquid preparations (such as, for example, suspensions, elixirs and solutions); or carriers such as starches, sugars, diluents, granulating agents, lubricants, binders, disintegrating agents and the like in the case of oral solid preparations (such as, for example, powders, capsules and tablets). Because of their ease in administration, tablets and capsules represent the most advantageous oral dosage unit form, in which case solid pharmaceutical carriers are obviously employed. If desired, tablets may be sugar-coated or enteric-coated by standard techniques.

The active agent is preferably administered in a therapeutically effective amount. An “effective amount” includes an amount of fructan or fructan containing product to promote an improvement in indicators of intestinal health or an improvement in indicators of severity of the condition such as diabetes, obesity, heart disease, hypertension, constipation, osteoporesis and cancer. The actual amount administered and the rate and time-course of administration will depend on the nature and severity of the condition being treated. Prescription of treatment, e.g. decisions on dosage, timing, etc. is within the responsibility of general practitioners or specialists and typically takes account of the disorder to be treated, the condition of the individual patient, the site of delivery, the method of administration and other factors known to practitioners. Examples of techniques and protocols can be found in Remington's Pharmaceutical Sciences, (supra).

The food or beverage or pharmaceutical preparation may be packaged ready for sale or in bulk form. The invention also provides methods of preparing the food, beverage or pharmaceutical preparation of the invention, and recipes or instructions for preparing such foods or beverages. The methods may comprise the steps of harvesting the plant or plant part, separating grain from other plant parts, crushing, extracting, milling, cooking, canning, packaging or other processing steps known in the art. The methods or recipes or instructions may include the steps of processing the plant product of the invention and/or admixing it with other food ingredients, such as heating or baking the mixture or the product to, for example, at least 100° C. The method may include the step of packaging the product so that it is ready for sale.

In some preferred but not essential embodiments the invention is directed to products and compositions for use in the herein described methods and does not extend to methods for the treatment of the human or animal body by surgery or therapy and disagnostic methods practiced on the human or animal body.

In some embodiments the invention is directed to the use of the subject products or compositions in the manufacture of a medicament for inter alia increasing intestinal health or, amealiorating one or more symptoms of a condition associated with low levels of dietary fructan, such as diabetes, obesity, heart disease, hypertension, constipation, osteoporesis and cancer.

### Industrial Use

The plant products, preferably grain, may be used in production of industrial products such as, for example, ethanol.

The present invention is further described by the following non-limiting Examples.

### EXAMPLES

### Example 1

### Illustrative Methods and Materials

### Plant Material

The mutant barley (Hordeum vulgare) lines M292 and M342, which were homozygous for a null mutation in the gene encoding SSIIa, were obtained following mutagenesis of grains of the barley variety ‘Himalaya’with sodium azide (Morell et al. 2003 (supra)). Mutant seeds were initially selected from progeny grain of the mutagenised population on the basis of a shrunken grain phenotype. This phenotype can be scored readily in a large population of mutagenized seed. The mutant lines were further characterised by their altered starch properties, reduced SSIIa protein level and activity, and genetically by the presence of a premature stop codon in the protein coding region of the gene encoding SSIIa (Morell et al. 2003 (supra)).

Both wild-type Himalaya and the mutant plants were grown in a controlled growth cabinet at day and night temperatures of 18° C. and 12° C. respectively with a 12 hour day-length. Barley spikes were labelled as at anthesis 2 days after the awns first appeared through the top of the flag leaf containing the enclosed spike. Developing seeds were harvested at 20 days post anthesis (DPA) and after removal of the embryo the developing endosperm was extruded through the cut surface and stored at −80° C.

Cereal cultivars and other varieties as described herein were obtained commercially or from the Australian Winter Cereals Collection, Tamworth, NSW, Australia. Crossing of plants such as barley plants was carried out in the greenhouse by standard methods.

### Grain Characteristics

Grain was harvested from plants at maturity. Average seed weight was determined by weighing 100 seeds and expressed as an average weight per grain (mg). Seed moisture content of grain was measured by standard nuclear magnetic resonance (NMR) methods using an Oxford 4000 NMR Magnet (Oxford analytical instruments Limited). Grain texture was measured using the Single-Kernel Characterization system 4100 (Perten Instruments Inc. Springfield, IL 62707 USA) using the RACI Cereal Chemistry Official testing method 12-01.

### Milling of Grain

Grain was ground to wholemeal that would pass through a 0.5 mm sieve, using a cyclonic mill (Cyclotec 1093, Tecator, Sweden). The wholemeal was then used for the analysis below.

### β-Glucan Analysis

α-glucan content was assayed as described in Megazyme Method (AACC32.23), using 20 mg of wholemeal for each of three replicate samples.

### Total Starch Analysis

Total starch content of grain was assayed as described in Megazyme Method (AACC76.13) using 20 mg of wholemeal for each of three replicate samples.

Wholemeal was obtained by milling grain. Starch was isolated from the wholemeal using the method of Schulman and Kammiovirta, 1991 (supra).

### Analysis of Starch Composition and Characteristics

Amylose and amylopectin contents in the starch of the grain, or the ratio of amylose to amylopectin, was determined by Sepharose CL-2B gel filtration as follows (Gel filtration method). Approximately 10 mg of total starch was dissolved in 3.0 ml of 1M NaOH and fractionated on the basis of molecular weight by chromatography on a Sepharose CL-2B column (Regina et al., 2006 (supra)). The amount of starch in each of the fractions from the column were measured using the Starch Assay Kit (Sigma) according to the suppliers instructions. The total amount of amylopectin (first peak, higher molecular weight) and amylose (second peak, lower molecular weight) was calculated and the ratio or contents determined.

The distribution of chain lengths in the amylopectin of the starch may be analysed by fluorophore assisted carbohydrate electrophoresis (FACE) using a capillary electrophoresis unit according to Morell et al., Electrophoresis, 19: 2603-2611, 1998, after debranching of the starch samples. For example, amylopectin chain length distribution may be measured using a P/ACE 5510 capillary electrophoresis system (Beckman Coulter, NSW Australia) with argon laser-induced fluorescence (LIF) detection. Molar difference plots may be generated by subtracting the normalized chain length distribution for modified starch from the normalized distribution for starch from an isogenic non modified control.

The gelatinisation temperature profiles of starch samples may be measured using a Pyris 1 differential scanning calorimeter (Perkin Elmer, Norwalk CT, USA). The viscosity of starch solutions may be measured on a Rapid-Visco-Analyser (RVA, Newport Scientific Pty Ltd, Warriewood, Sydney), using conditions as reported by Batey et al., J. Sci. Food Agric. 74: 503-508, 1997. The parameters that may be measured include peak viscosity (the maximum hot paste viscosity), holding strength, final viscosity and pasting temperature. Pasting properties may be measured using the Rapid Visco Analyser as follows. Starch (3.0g) is added to distilled water (25.0 ml) in the DSC pan and the RVA run profile is: 2 mins at 50° C., heat for 6 mins to 95° C., hold at 95° C. for 4 mins, cool for 4 mins to 50° C., hold at 50° C. for 4 mins. The measured parameters are: Peak viscosity at 95° C., Holding strength at end of 95° C. holding period, Breakdown=Peak Viscosity—Holding strength, Final viscosity at end of 50° C. holding period, Setback=Final Viscosity—Holding strength. The software Thermocline for Windows version 2.2 (Newport Scientific Pty Ltd, NSW Australia) may be used for collection and analysis of data.

The swelling volume of flour or starch may be determined according to the method of Konik-Rose et al. Starch/die Starke 53:14-20, 2001. The uptake of water is measured by weighing the sample prior to and after mixing the flour or starch sample in water at defined temperatures (for example, 90° C.) and following collection of the gelatinized material.

### Lipid Analysis

Total lipid content was assayed by NMR using an Oxford 4000 NMR Magnet, Oxford Analytical Instruments Limited, UK. For each sample, 1 g of seeds was dried at 38.8° C. for 64 hours. The dried seeds were then measured using NMR and compared against a pure barley oil controls extracted from cv. Himalaya or M292 grain.

### Protein Analysis

Protein content was estimated by determining the total nitrogen content of the seed using the method of Dumas (RACI Method 02-03, 2003) and expressing the result as “protein” by multiplying the value obtained by a factor of 5.7. For each sample 10 mg of wholemeal was used and the nitrogen content was detected by mass spectrometry.

### Total Dietary Fibre Assay

The gravimetric method of Prosky et al., J Assoc Off Agric Chem 68: 677, 1985 was used to determine total dietary fibre (TDF) of the wholemeal. Duplicate samples were assayed.

### Non Starch Polysaccharide Assay

Total neutral non-starch polysaccharides (NSP) were measured by a modification of the gas chromatographic procedure of Theander et al., JAOAC Int 78: 1030-1044, 1995. The modification involved a 2-hour hydrolysis with 1 M sulphuric acid followed by centrifugation to remove insoluble NSP and a further hydrolysis of the supernatant using 2 M trifluoroacetic acid for soluble NSP.

### Resistant Starch Assay

An in vitro procedure was used to determine resistant starch (RS) content. The method has two sections: firstly, starch in each sample was hydrolysed under simulated physiological conditions; secondly, by-products were removed through washing and the residual starch determined after homogenization and drying of the sample. Starch quantitated at the end of the digestion treatment represented the resistant starch content of the sample. Typically, triplicate samples of whole meal along with appropriate standards were mixed with artificial saliva and the resultant bolus incubated with pancreatic and gastric enzymes at physiological pH and temperature. The amount of residual starch in the digesta was determined using conventional enzymatic techniques and spectrophotometry and the resistant starch content of the sample expressed as a percentage of sample weight or total starch content.

On day 1, an amount of sample representing up to 500 mg of carbohydrate was weighed into a 125 mL Erlenmeyer flask. A carbonate buffer was prepared by dissolving 121 mg of NaHCO₃ and 157 mg of KCl in approximately 90 mL purified water, adding 159 μL of 1 M CaCl₂.6H₂O solution and 41 μL of 0.49 M MgCl₂.6H₂O, adjusting the pH to 7 to 7.1 with 0.32 M HCl, and adjusting the volume to 100 mL. This buffer was stored at 4° C. for up to five days. An artificial saliva solution containing 250 units of α-amylase (Sigma A-3176 Type VI-B from porcine pancreas) per mL of the carbonate buffer was prepared. An amount of the artificial saliva solution, approximately equal to the weight of food, was added to the flask. About 15-20 sec after adding the saliva, 5 mL of pepsin solution in HCl (1 mg/mL pepsin (Sigma) in 0.02 M HCl, pH 2.0, made up on day of use) was added to each flask. The mixing of the amylase and then pepsin mimicked a human chewing the sample before swallowing it. The mixture was incubated at 37° C. for 30 min with shaking at 85 rpm. The mixture was then neutralised with 5 mL of 0.02M NaOH. 25 mL of acetate buffer (0.2 M, pH 6) and 5 mL of pancreatin enzyme mixture containing 2 mg/mL pancreatin (Sigma, porcine pancreas at 4× USP activity) and 28U of amyloglucosidase (AMG, Sigma) from Aspergillus niger in acetate buffer, pH6, were added per flask. Each flask was capped with aluminium foil and incubated at 37° C. for 16 hours in a reciprocating water bath set to 85 rpm.

On day 2, the contents of each flask was transferred quantitatively to a 50 mL polypropylene tube and centrifuged at 2000×g for 10 min at room temperature. The supernatants were discarded and each pellet washed three times with 20 mL of water, gently vortexing the tube with each wash to break up the pellet, followed by centrifugation. 50 μL of the last water wash was tested with Glucose Trinder reagent for the absence of free glucose. Each pellet was then resuspended in approximately 6 mL of purified water and homogenised three times for 10 seconds using an Ultra Turrax TP18/10 with an S25N-8G dispersing tool. The contents were quantitatively transferred to a 25 mL volumetric flask and made to volume. The contents were mixed thoroughly and returned to the polypropylene tube. A 5 mL sample of each suspension was transferred to a 25 mL culture tube and immediately shell frozen in liquid nitrogen and freeze dried.

On day 3, total starch in each sample was measured using reagents supplied in the Megazyme Total Starch Procedure kit. Starch standards (Regular Maize Starch, Sigma S-5296) and an assay reagent blank were prepared. Samples, controls and reagent blanks were wet with 0.4 mL of 80% ethanol to aid dispersion, followed by vortexing. Immediately, 2 mL of DMSO was added and solutions mixed by vortexing. The tubes were placed in a boiling water bath for 5 min, and 3 mL of thermostable α-amylase (100 U/ml) in MOPS buffer (pH 7, containing 5 mM CaCl₂ and 0.02% sodium azide added immediately. Solutions were incubated in the boiling water bath for a further 12 min, with vortex mixing at 3 min intervals. Tubes were then placed in a 50° C. water bath and 4 mL of sodium acetate buffer (200 mM, pH 4.5, containing 0.02% sodium azide) and 0.1 mL of amyloglucosidase at 300 U/ml added. The mixtures were incubated at 50° C. for 30 min with gentle mixing at 10 min intervals. The volumes were made up to 25 mL in a volumetric flask and mixed well. Aliquots were centrifuged at 2000×g for 10 min. The amount of glucose in 50 μL of supernatant was determined with 1.0 mL of Glucose Trinder reagent and measuring the absorbance at 505 nm after incubation of the tubes at room temperature in the dark for a minimum of 18 min and a maximum of 45 min.

### Quantification of Sucrose, Hexoses and Fructo-Oligosaccharides

Total sugars were extracted from wholemeal following the method of Lunn and Hatch, Planta 197: 385-391, 1995 with the following modification. Wholemeal such as barley wholemeal (100 mg) was extracted 3 times with 10 ml of 80% ethanol (v/v) in a boiling water bath for 10 minutes. The supernatant from each extraction was pooled and freeze dried, then re-suspended in 2 ml milliQ water. The quantities of sucrose, glucose, and fructose were measured using a colorimetric microtiter plate enzymatic assay as described (Campbell et al., Journal of the science offood and agriculture 79: 232-236, 1999; Ruuska et al., Functional Plant Biology 33: 799-809, 2006). Sugars and fructo-oligosaccharides were also analysed by high performance anion exchange chromatography (HPAEC) as described in Ruuska et al., 2006 (supra); both methods resulted in comparable values.

To determine maltose levels, total sugars extracted from barley whole meal were assayed essentially as described by Bernfeld, In: Colowick S, Kaplan N (eds), Methods in enzymology. Academic, NY, p 149, 1955, using maltose standard solutions for comparison, as follows. Total sugars were diluted 10 to 100-fold. Maltose standards (10 tubes) were prepared as 0.3 to 5 micromoles per ml. One ml of each dilution of maltose (in total sugars or maltose dilutions) was mixed with 1 ml of dinitrosalicylic acid colour reagent. The sugar solution was then incubated at 100° C. for 5 minutes and cooled to room temperature. Ten ml reagent grade water was added to each tube and mixed well. The samples were measured at A₅₄₀ with a spectrophotometer. Maltose was also determined by HPAEC as described above.

### Enzyme Assays

Total starch synthase activity in samples such as developing endosperm of cereals may be measured by extraction of proteins and assay by the methods described in Libessart et al. Plant Cell 7(8): 1117-1127, 1995 or Cao et al., Plant Physiol. 120: 205-215, 1999. The assays use ¹⁴C labeled ADPG substrate and measure incorporation of the monomer into starch polymers. Individual isoforms of starch synthase in extracts may be separated by gel electrophoresis and assayed in-gel (zymogram) as follows. Extracts from samples such as developing seeds may be prepared using 50 mM potassium phosphate buffer, pH7.5, 5 mM EDTA, 20% glycerol, 10 μM Pefabloc and 0.05 mM dithiothreitol (DTT). After grinding the seeds to a pulp in the buffer or homogenizing the sample, the mixture is centrifuged at 14,000g for 15 min at 4° C. and the supernatant drawn off. The protein concentration in the supernatant may be measured using Coomassie Protein Reagent or other standard means. Extracts may be stored at −80° C. if the protein extracts are to be run on native gels. For denaturing gel electrophoresis, 100 μl of extract is mixed with SDS and β-mercaptoethanol and the mixtures are incubated in boiling water for 4 min to denature the proteins. Electrophoresis is carried out in standard denaturing polyacrylamide gels using 8% polyacrylamide separating gels overlaid with 4.5% polyacrylamide stacking gels. After electophoresis, the proteins may be renatured by soaking the gels in 40 mM Tris-HCl buffers for a minimum of 2 hr, changing the buffer every 30 min and using at least 100 mL of buffer for each buffer change. For non-denaturing gels, the denaturing step with SDS and β-mercaptoethanol is omitted and SDS omitted from the gels. A starch synthase assay buffer including Tris-glycine (25 mM Tris, 0.19M glycine), 0.133M ammonium sulphate, 10 mM MgCl₂, 670 μg/mL BSA and 1 mM ADPG substrate may be used to detect starch synthase bands, followed by staining with 2% KI, 0.2% I2 iodine solution to detect the starch product.

Alternatively, starch synthase or other starch biosynthetic enzymes may be detected in samples using specific antibodies (ELISA).

cDNA array

The New South Wales Centre for Agricultural Genomics (NSWCAG) array contained 19,635 wheat cDNA clones and 1,613 barley cDNA clones of which about 16,000 were unique in nucleotide sequence, wherein a “unique” sequence is defined herein as having less than 80% sequence identity at the nucleotide level to all of the other sequences. The design of this array and the cDNA libraries that contributed to its construction are detailed in Clarke and Rahman, Theoretical and Applied Genetics, 110: 1259-1267, 2005.

### Extraction of RNA

To extract total RNA from developing endosperm, samples of eight endosperms each were frozen in liquid N₂ and ground to a fine powder with the aid of acid washed sand in a mortar containing 2 ml of NTES (0.1 M NaCl, 10 mM Tris-HCl (pH 8.0), 1 mM EDTA, 1% (w/v) SDS). Two ml of phenol/chloroform was added to the homogenized mixture and again ground well. This mixture was transferred to a capped tube, vortexed for one minute and incubated on ice for 30 minutes. The two phases were separated by centrifuging the tubes at 5,500g for 15 minutes at 4° C., the aqueous phase transferred to a new tube and an equal volume of 4 M LiCl/10 mM EDTA added to precipitate the RNA. After thorough mixing, the samples were kept at −20° C. over night. Samples were thawed and spun at 7,000g for 45 minutes at 4° C. The supernatant was discarded and the pellet containing the RNA rinsed with 1 ml of 70% ethanol, dried and re-suspended in 250 μl of water. The soluble fraction was transferred to a new microcentrifuge tube. To remove starch contamination from the isolated RNA, 3.5 μl of 3 M NaOAc and 125 μl of ethanol (EtOH) was added followed by mixing and centrifugation at 13,000 rpm for 10 min at 4° C. The supernatant was removed to a new tube and 21.5 μl of 3 M NaOAc and 375 μl of EtOH added to precipitate the RNA. The precipitated RNA was rinsed with 70% EtOH then dried and re-suspended in 30 μl of water. To determine the concentration and purity of the RNA, aliquots were diluted 1 in 300 in sterile distilled water and the absorbance measured at A₂₆₀ and A₂₈₀.

### Microarray Analysis

For microarray analysis, four biological replicates were used for the mRNA comparisons. For each labeling experiment, 50 g of total RNA was used for both the Cy3 and Cy5 dyes (Amersham Pharmacia, UK), following the two-step labelling method of Schenk et al., Proc. Natd. Acad. Sci. U.S.A. 97: 11655-11660, 2000. RNA from the control plant, ‘Himalaya’was labelled with Cy3 in the un-swapped replicates. The dye labelling of the samples was reversed for two of the replicates to minimize any bias in cDNA incorporation and photo-bleaching of the fluorescent dyes. The pre-hybridization of the microarray slides, hybridization of samples and subsequent washing of the slides to remove unbound target was performed as per the supplied protocol for the CMT-GAPS™ coated microscope slides (Corning USA). The slides were scanned with a GenePix 4000A microarray scanner (Axon Instruments, Union, CA, USA). The features were analyzed using the GenePix Pro 4 software and unsatisfactorily segmented features were either manually adjusted or discarded to ensure the integrity of the data obtained.

The analysis of the microarray data files was carried out using functions contained in tRMA (tools for R Microarray Analysis; Wilson et al., Bioinformatics 19: 1325-1332, 2003) these functions operate as part of a statistical software package called R (http://www.r-project.org/). A detailed description of tRMA is available online (www.csiro.au/gena/trma). The data sets generated from the GenePix software were loaded into tRMA using the “LoadGenePixFile” function. Normalization of log₂ ratio values was performed using the “SpatiallyNormalize” function. This method of normalization corrected for spatial and intensity-dependent effects of fluorescence across the microarray slide (Wilson et al., 2003 (supra)). In addition, the possible biases in fluorescence due to differences in the efficiency of incorporation of the two dyes and unequal loading of cDNA samples were also corrected. Using the median values of the normalized log ratios for each gene in each replicate, differentially expressed genes were determined using the “FindDiffExpGenes” function at a stringency level of 1e-10. Differentially expressed genes are selected as outliers in a Gaussian distribution of the entire data set. Therefore, a ratio cut-off was empirically computed from the normalized log₂ ratio data, which were rescaled and centered in order to make direct comparisons between slides in all four replicates. The same slide data was also compared using a different function. In this analysis after slide normalization the “FindDiffExpGenes” function was performed on the individual slides at the default stringency (1e-3). Then “ComparelnterestingGenes” function was used to find the differentially expressed genes common to all replicates.

To obtain the nucleotide sequence of cloned genes or, for example, to confirm the identity of the differentially expressed genes, the clones were sequenced using 0.12 g of PCR amplified insert as the template, from both the 3′ and 5′ ends using primers such as M13/pUC reverse and forward primers and Big Dye Terminator Cycle Sequencing (ABI).

### RNA Electrophoresis and Hybridization Conditions

For each sample, 10 g of total RNA was separated in a 1.4% agarose-formaldehyde gel (w/v) and transferred to Hybond N+ membrane (Amersham Pharmacia Biotech UK Ltd.) using the standard alkali transfer protocol supplied by the manufacturer.

Probes used for RNA hybridizations were made by amplifying the inserts from cDNA clones. PCR SuperMix (Invitrogen) was used with 3 ρmoles of each primer and 50 ng template DNA in 10 al reaction mix. The probes were amplified under the following conditions; cycle 1, 94° C. for 5 minutes; cycle 2, 94° C. for 30 seconds, 55° C. for 30 seconds and 72° C. for 2 minutes, repeat cycle 2 for 35 cycles. The inserts were labeled using the “Megaprimet™ DNA labelling system” (Amersham Biosciences). In addition the clone pTa250.2, containing the coding sequences of the ribosomal genes (Appels and Dvorak, Theoretical and Applied Genetics, 63: 337-348, 1982) was used to estimate the uniformity of loading for the RNA from Himalaya and M292 onto the gels.

Hybridization of probes was at 65° C. in Khandjian hybridization buffer (Khandjian, Bio/Technology, 5: 165-167, 1987). The membrane was washed once for 30 minutes at 65° C. with 2×SSC and 0.1% (w/v) SDS, and twice for 40 minutes at 65° C. with 0.2×SSC and 0.1% (w/v) SDS, which corresponds to a high stringency wash. The membrane was exposed using a Fujifilm FLA-5000 series fluorescent image analyzer system and the image obtained was analyzed using the Multi Gauge version-2 analysis software (Fuji Photo Film Co. Ltd., 26-30 Nishiazabu, 2-Chome Minato-ku Tokyo 106-8620, Japan). The variation in hybridization intensities between the Himalaya and M292 RNA samples was measured (pixels per mm²) and a correction was made for the level of background hybridization to the gel lanes.

### Example 2

### Composition and Functional Parameters of M292 Barley Grain

A detailed analysis was undertaken of the composition of mature grain from the barley SSIIa mutant M292 with a comparison to wild-type grain (Himalaya) grown under the same conditions. The results are summarized in Table 1.

M292 producing thinner grains with an unfilled central region (“shrunken”). The moisture content of the M292 grain was 10.2% as measured by the NMR method compared to 10.6% for Himalaya. The average grain weight for M292 was 36.4 mg/grain compared to Himalaya at 46.3 mg/grain, which represents a 21% reduction for M292, primarily due to reduction in the starch content from 27.7 mg/grain to 10.6 mg/grain (62% reduction). The reduction in starch came mostly from less amylopectin (84% reduction) while the amylose level was reduced by only 25%. Consequently, the percentage starch as amylose increased from about 37% in the wild-type to about 73% in M292 as determined by the gel filtration method.

TABLE 1
Grain composition of barley M292 at maturity

| | Content in | | | | Ratio |
|---|---|---|---|---|---|
| | Himalaya | | Content in M292 | | M292/Him. |
| | | (% of total | | (% of total | (on a mg per |
| | (mg/grain) | grain) | (mg/grain) | grain) | grain basis) |
| Seed weight | 46.3 | | 36.4 | | 0.79 |
| Starch | 27.7 | 59.9 | 10.6 | 29.1 | 0.38 |
| amylose | 10.4 | 26.1 | 7.8 | 21.4 | 0.75 |
| amylopectin | 17.3 | 43.6 | 2.8 | 7.7 | 0.16 |
| Protein | 5.9 | 12.8 | 7.0 | 19.3 | 1.2 |
| Total NSP | 5.6 | 12.1 | 7.4 | 20.3 | 1.3 |
| Lipid | 1.5 | 3.2 | 2.5 | 6.9 | 1.7 |
| Sugars (total) | 0.07 | 0.16 | 0.6 | 1.6 | 8.6 |
| glucose | 0.005 | | 0.08 | | 16.0 |
| fructose | 0.005 | | 0.09 | | 18.0 |
| sucrose | 0.060 | | 0.39 | | 6.5 |
| maltose | 0.005 | | 0.03 | | 6.0 |
| Fructan | 0.1 | 0.2 | 4.2 | 11.5 | 42.0 |
| Ash | 1.1 | 2.4 | 0.8 | 2.2 | 0.73 |
| Moisture | 4.9 | 10.6 | 3.7 | 10.2 | |
| Total content | 47.4 | 102.4 | 36.8 | 101.1 | |
| (incl. water) | | | | | |
| Resistant starch | 0.3 | 0.7 | 1.3 | 3.6 | 4.3 |
| Beta-glucan | 2.7 | 5.8 | 3.6 | 9.9 | 1.3 |
| Dietary fibre | 7.2 | 15.6 | 9.2 | 25.3 | 1.3 |
| Arabinoxylan | 2.5 | 5.5 | 3.1 | 8.6 | 1.2 |

Note:
Total content is a sum of components in bold type; NSP: Non starch polysaccharide.

The reduced starch amount in M292 was compensated for in part by increases in the amounts of non-starch components. The contents of protein, total NSP and lipid (measured as mg per grain), which together make up about 46% of the grain weight of M292, were increased by 1.2, 1.3 and 1.7-fold, respectively. As a percentage of total M292 grain weight, the increases were even greater. The levels of free sugars were increased in M292 by about 8-fold in total, with individual sugars increasing between 6—and 18-fold. Of the other carbohydrate components, α-glucan and arabinoxylans were increased in the mutant by 1.3 and 1.2-fold, respectively. Unexpectedly and surprisingly, fructo-oligosaccharides (fructans) were increased massively by 42.0-fold. The extent of this increase was most surprising since α-glucan and arabinoxylans, which are also polymeric carbohydrates, were increased only modestly. The increase for fructan in terms of percentage of total grain weight was from 0.2% for Himalaya to 11.5% for M292. It is believed such a high level of fructan has never before been seen in a grain.

The total carbohydrate content (including starch, total NSP, free sugar and fructan) was reduced from 33.5 mg/grain present in wild-type Himalaya grain to 22.8 mg/grain in M292. The amount of resistant starch (RS) as determined by the in vitro method as described in Example 1 was increased in M292 by 4.3 fold (mg per grain). This indicated a much increased extent of protection of the starch from amylase digestion, presumably due to an altered starch granule structure and related to the increased proportion of amylose in the starch. Increased levels of lipid, α-glucan and fructan are also thought to have contributed to the increased level of RS.

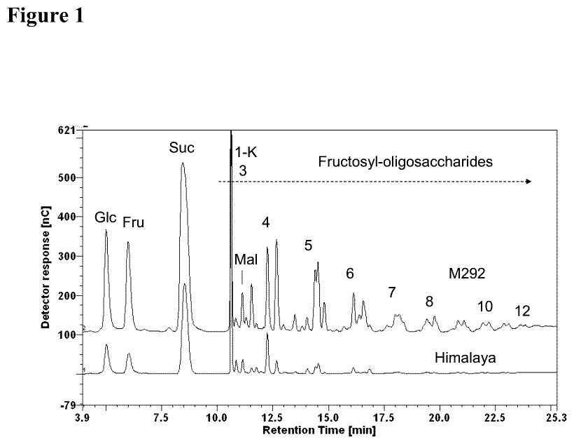

The composition of the soluble carbohydrate components was further examined by HPAEC to study the changes in the oligosaccharides. The chromatography profiles (FIG. 1) confirmed the increased content of sucrose, maltose and hexoses in M292, as well as increased levels of a range of fructo-oligosaccharides, which showed a degree of polymerization (DP) of from 3 up to about 12. The wild-type Himalaya grain contained negligible or undetectable levels of fructo-oligosaccharides having a DP of about 6 or above.

Changes in cell wall polysaccharides can affect the hardness of the mature barley grain (Tsuchiya et al., Physiologia Plantarum 125: 181-191, 2005; Fincher and Stone, Advances in Cereal Science and Technol 8: 207-295,1986). As there were significant changes of total NSP in M292, grain hardness was measured to determine the level of changes. The measurements were taken using the Single-Kernel Characterization System (SKCS) to obtain an average hardness index (HI) based on measurements from 300 grains. For M292 the HI value was 109±15 and the HI value for Himalaya was 84±17, indicating an increase in grain hardness for the mutant.

The contents of protein, total NSP and lipid (measured as mg per grain) which together make up about half the grain weight of M292, were increased in absolute amount and as a percentage of total grain weight, by a 1.2, 1.3 and 1.7-fold change respectively. Of the carbohydrate components which may be particularly significant in a nutritional context, α-glucan, arabinoxylans and fructo-oligosaccharides, were increased in the mutant by, 1.3-, 1.2-, and 42.0-fold, respectively. The resistant starch (the proportion of the total starch that is resistant to digestion in the human small intestine) was also increased in M292 by 4.3 fold change when expressed as mg per grain. The total content of carbohydrates (including starch, total NSP, free sugar and fructan) in M292 reduced to 22.8 mg compared to 33.5 mg present in wild-type Himalaya on a mg per grain basis.

### DISCUSSION

The identification and initial characterization of the barley M292 mutant was described by Morell et al. 2003 (supra) and Topping et al., 2003 (supra). Using linkage analysis and subsequent sequencing of the candidate gene, Morell et al. 2003 (supra) demonstrated that the mutation was caused by a stop codon introduced into SSIIa and resulted in a shrunken grain in which the seed weight was reduced from an average of 46 mg in Himalaya to 36 mg in M292 (Table 1) producing a thinner seed with an unfilled central region. The decreased starch, grain weight and modified starch composition observed here were consistent with previous studies (Morell et al. 2003 (supra)). The inventors have also shown increased levels of protein, total NSP, and lipid both on an individual grain basis and as a percentage, consistent with determinations reported earlier (percentage of total grain basis, Bird et al., 2004a (supra)).

In addition, the inventors have also shown, for the first, time significantly increased levels of free sugars and fructo-oligosaccharides. As well as increased resistant starch, β-glucan, and dietary fibre, the higher fructo-oligosaccharides determined here are expected to be important for providing beneficial dietary outcomes.

### Example 3

Identification of Differentially Expressed Genes Using a cDNA Microarray

A microarray containing 19,635 wheat clones and 1,613 barley clones of which about 16,000 were unique sequences was obtained from the New South Wales Centre for Agricultural Genomics (NSWCAG). A “unique” sequence is defined herein as a sequence having less than 80% sequence identity at the nucleotide level to all other sequences in the set. The design of this array and the cDNA libraries that contributed to its construction are detailed in Clarke and Rahman, 2005 (supra).

The array was hybridized using total RNA from developing endosperm (20 DPA) of M292 or the wild type ‘Himalaya’(control). At this time point, the levels of starch and protein were increasing in the developing barley grain. It was expected that the transcriptional changes to the starch biosynthetic pathway, caused by the mutation in the SSIIa gene, would be most evident at this timepoint. Seeds at the same age and morphological stage of development were selected from a single spike to represent a sample. The seeds collected from a different spike represented a replicate biological sample.

Four biological replicate experiments for M292 and the control Himalaya were used to compare the transcription profiles in developing endosperm at the mid grain fill stage. Differentially expressed genes were identified from the median data sets of differentially expressed genes from the four microarray experiments. The stringency level used to select the genes was 1e-10. This level of stringency was determined empirically to give the least number of false positive results. From the tRMA analysis, 42 array features were identified as differentially expressed, 20 of which were up-regulated and 22 down regulated in M292 compared to Himalaya. All of the clones identified were verified by sequence analysis from both the 3 prime and 5 prime ends to ensure that the annotation of the clone was correct. These results showed that one clone was a chimera and this clone was not analyzed further. Using the sequence data, a sequence homology search was made against the TIGR (http://www.tigr.org) tentative consensus sequence (TC) data base using either the barley or wheat Gene Index, depending on the origin of the clone on the microarray. From this comparison the 41 cDNA clones could be grouped into 23 different genes. A verification of the expression changes was then undertaken by RNA blot-hybridization using one clone as a representative for each TC. The expression changes were verified using an RNA gel blot analysis in which RNA was isolated from a fifth replicate endosperm sample. The change in expression for 6 clones was not confirmed and these clones were not analyzed further.

The 17 clones which passed the quality control conditions, their TC sequence and the expression change, relative to Himalaya, for both the microarray and RNA gel blot analysis are listed in Table 2. These genes have been grouped into four categories, being carbohydrate related, defense, stress response and those genes for which a biological function has not yet been established. In Table 2, column 4 presents the microarray results (M292/Himalaya) and column 5 presents the RNA gel blot analysis results. Therefore, the numbers in these columns represent the fold change in gene expression in M292 above or below the Himalaya control. Clone pTa250 (Appels and Dvorak, 1982 (supra)) was used as a control sequence for the RNA blot hybridizations.

TABLE 2
Differentially expressed genes in M292 developing grain

| | | | | |
|---|---|---|---|---|
| TIGR TC | GenBank ID | Name | MicroArray | Northern |

Carbohydrate related genes

| | | | | |
|---|---|---|---|---|
| 250803 | AL812383 | β-D-glucan exohydrolase | 2.33 | 4.60 |
| 250388 | BQ607866 | sucrose synthase | 2.47 | 1.25 |
| 139354 | CV054497 | serpin | 0.30 | 0.10 |
| 249933 | BQ609223 | β-amylase | 0.27 | 0.20 |
| 269042 | BQ606784 | starch synthase | 0.17 | 0.03 |

Defense related gene

| | | | | |
|---|---|---|---|---|
| 146614 | CV055257 | alpha-amylase inhibitor BDAI-I | 0.36 | 0.53 |

Stress protein related genes

| | | | | |
|---|---|---|---|---|
| 232242 | BG263730 | t.complex protein | 2.56 | 2.10 |
| 246814 | AL818443 | dnaK-type protein | 2.30 | 1.60 |
| 250385 | BG314518 | heat shock protein 80 | 2.27 | 3.22 |
| 264292 | BE442600 | heat shock protein 70 | 2.25 | 2.00 |
| 139615 | CV056993 | prohibitin | 2.04 | 1.70 |

Genes of unassigned function

| | | | | |
|---|---|---|---|---|
| 246973 | BQ606826 | annexin p33 | 2.85 | 4.10 |
| 232395 | X83881 | S-adenosylmethionine decarboxylase | 2.37 | 1.30 |
| 233204 | BE444846 | Rubber elongation factor | 2.14 | 3.32 |
| 249807 | BQ608029 | O.s r40g2 protein | 2.26 | 10.00 |
| 234638 | BQ606719 | Puro/hordoindoline-a | 0.40 | 0.58 |
| 147907 | CV060362 | No Hit either blast x or n | 0.32 | 0.27 |

Interestingly, many genes known to play a role in starch biosynthesis that were represented on the microarray did not show differential expression in M292 compared to Himalaya. These were as follows (with Genbank Accession Nos.): ADP Glucose pyrophosphorylase small subunit (AL815034); ADP Glucose pyrophosphorylase large subunit (AL814437); Granule bound starch synthase I (BQ608470); Granule bound starch synthase II (BE497955); Starch synthase I (AL815975); Starch synthase III (AL811419); Starch branching enzyme I (AL816520); Starch branching enzyme IIa (AL812818); Starch branching enzyme IIb (BF201559); Isoamylase: glycogen 6-glucanohydrolase (BE422551); Alpha amylase (AL809888).

At 20 DPA in barley grain, 4 differentially expressed genes other than SSIIa were identified that have been related to the storage of carbon through sucrose uptake in the endosperm. Of these 4 genes, 2 were up regulated in M292 (β-D-glucan exohydrolase and sucrose synthase) and 2 down regulated (serpin, β-amylase and ssIIa).

The SSIIa transcript was almost undetectable by RNA gel blot analysis, being reduced to only 3% of the wild-type level. This result was consistent with the absence of SSIIa proteins in the M292 developing endosperm and mature grain as observed by Morell et al. 2003 (supra). The reduction by 97% may be a consequence of rapid turnover of the mRNA by the nonsense-mediated mRNA decay pathway. The smaller reduction of ssIIa transcript observed in the microarray experiment compared to the RNA gel blot hybridization result, may have been due to some cross hybridization of transcripts from other members of the starch synthase families. This example shows the importance of confirming results obtained from the arrays with RNA gel blot hybridization or RT-PCR experiments, as these methods can be targeted to specific members of the gene families.

The function of β-amylase in the germinating grain is to remove successive maltose units from the non reducing ends of the starch chain to provide a source of carbon, but it is also synthesized and accumulates during grain development (MacGregor et al., Cereal Chemistry, 48: 255-269, 1971). Guerin et al., Journal of Cereal Science, 15: 5-14, 1992 showed that β-amylase was present in the seed as a bound inactive complex with Protein Z (serpin) that is associated with the starch granules. In this study the transcript levels of both serpin (protein Z type) and β-amylase were reduced in the mutant line indicating that these proteins may be co-regulated in the developing grain.

The expression of sucrose synthase (SuS) was slightly up-regulated in M292 endosperm. The catalysis of sucrose and UDP to form UDP-glucose and fructose is carried out by SuS in a reversible reaction. The UDP-glucose produced by SuS can be converted via the combined action of UDP-glucose pyrophosphorylase and phosphoglucomutase to give Gluc-1-P and Gluc-6-P which can enter glycolysis or be used in starch synthesis (Winter and Huber, Critical Reviews in Plant Sciences 19: 31-67, 2000). Alternatively, it can provide the substrate for the synthesis of cellulose, callose, or other cell wall polysaccharides. It appears that the mutation ssIIa in M292 disrupted the utilization of sucrose for starch synthesis, associated with an increase in the level of sucrose (6.5 fold increased in M292, Table 1) and increased SuS expression.

Other sucrose regulated, or UDP-glucose utilizing enzymes were not differentially expressed in this analysis. For example, there were 11 clones present on the cDNA array relating to a range of cellulose synthase or cellulose synthase-like genes and 3 callose synthase genes, none of which were differentially expressed. However, there was a significant increase (33% on a mg per grain basis) of β-glucan content in the M292 grain and UDP-glucose is a substrate for the synthesis of this polysaccharide. While the level of fructo-oligosaccharides were greater in mature M292 grain, than in Himalaya, genes encoding enzymes of fructan biosynthesis, such as sucrose:sucrose 1-fructosyltransferase and sucrose:fructan 6 fructosyltransferase, were not differentially expressed in this comparison.

There was an increase in expression of the gene β-D-glucan exohydrolase in M292. This exohydrolase has a broad range of substrate specificity which presents a problem in assigning a specific target molecule for this enzyme. Hrmova and Fincher, Plant Molecular Biology, 47: 73-91, 2001 suggested the β-D-glucan exohydrolases be classified as polysaccharide exohydrolases because they can hydrolyze a range of polysaccharides and oligosaccharides. This enzyme is usually found in situations where cell wall degradation or modification is occurring (Harvey et al., Physiologia Plantarum 113: 108-120, 2001; Hrmova and Fincher, 2001 (supra)). A possible role for this enzyme in M292 is the degradation of those endosperm cells that are not filled in the shrunken mutant grain, or recovering/recycling glucose as proposed by Hrmova and Fincher, 2001 (supra).

Previous analysis showed that the quantities of starch branching enzymes IIa, IIb are similar in the wild type and M292 seed (Morell et al. 2003 (supra)). The observation that the protein level was unchanged is consistent with the present transcription analysis where no differential expression was observed for branching enzyme IIa or IIb.

The role of the other differentially expressed genes identified in this analysis in M292 is unknown.

In summary, in wild-type Himalaya sucrose transported into the grain can be efficiently converted to storage carbohydrates. Two major forms of the carbohydrates in Himalaya are starch (82.8% of total carbohydrate) and NSP (16.7% of total carbohydrate), with free sugar and fructan being the minor components. In M292, due to the ssIIa mutation, this process has been disrupted, leading to the reduction of total starch to 46.6% of total carbohydrate. It is proposed that the ssIIa mutation leads to the accumulation of sucrose that up-regulates the expression of SuS. The sucrose and UDP-glucose (synthesized by SuS) are then used as the substrates for the synthesis of NSP (32.5% of total carbohydrate), fructan (18.4% of total carbohydrate) and free sugar (2.6% of total carbohydrate) instead of starch. The differential expression of UDP-glucose pyrophosphorylase was not detected, although this cDNA was on the microarray.

### Example 4

### Determination of Fructan Content in Grain

### Method for Quantification of Fructo-Oligosaccharides (Fructans)

Total sugars were extracted from wholemeal following the method of Lunn and Hatch, 1995 (supra) with the following modification. Wholemeal is defined herein as the product obtained by milling mature grain, without subsequent fractionation (e.g. sieving) to remove the bran. Therefore wholemeal contains all of the components in the grain.

The wholemeal was extracted 3 times with 10 ml of 80% ethanol (v/v) in a boiling water bath for 10 minutes. The supernatant from each extraction was pooled and freeze dried, then re-suspended in 2 ml milliQ water. The quantities of sucrose, glucose, and fructose were measured using a colorimetric microtiter plate enzymatic assay as previously described (Campbell et al., 1999 (supra); Ruuska et al., 2006 (supra)). Sugars and fructo-oligosaccharides (fructans) were also analysed by high performance anion exchange chromatography (HPAEC) as described in Ruuska et al., 2006 (supra). Both methods gave similar results.

### Comparison of Fructan Contents Among Barley Varieties

Barley lines with different genetic backgrounds were used in a comparison of fructan contents. The barley lines used were: (1) lines that contain a starch synthase IIa (SSIIa) mutation (Barley M292, Barley M342 and Tantangara x 292 double haploid (DH)); (2) a barley line with a waxy mutation inactivating GBBSI (Waxiro); (3) a high amylose barley (with 45% amylose, and a mutated gene(s) designated amol); and (4) wild-type barley lines (Gardiner, Schooner, Himalaya, Sloop, Namoi and Glacier). These lines were grown at Francis, South Australia. Tantangara x 292 DH is a barley line from a double haploid population from the crossing between Tantangara and Barley M292.

The results are shown in Table 3, where the fructan content represents the sum amount of glucose and fructose after hydrolysis minus the amounts of free glucose, fructose and sucrose (without hydrolysis). In a completely unexpected result, the data indicated that the barley lines containing a mutation in the SSIIa that inactivated SSIIa produced relatively high levels of fructan (shown in mg/g grain), whereas the barley lines without the SSIIa mutation produced relatively low levels of fructan. The observation was even more surprising in view of the lack of any change in the expression levels of two genes involved in fructan biosynthesis (Example 3).

TABLE 3
Analysis of fructan levels in grain of barley varieties

| | Un-hydrolysed | | | | | | |
|---|---|---|---|---|---|---|---|
| | Glucose | Fructose | Sucrose | | Hydrolysed | | |
| | content | content | content | Maltose | Glucose | Fructose | Fructan |
| Barley line | (mg hexose equiv/g dry weight) | | | content | content | content | content^{a} |
| Barley M292 | 2.3 | 2.4 | 10.6 | 0.9 | 41.2 | 75.0 | 100.1 |
| | | | | | 49.0 | 97.9 | 130.7 |
| Gardiner | 0.6 | 0.7 | 6.5 | 0.3 | 15.9 | 28.1 | 35.8 |
| | | | | | 10.6 | 21.4 | 23.8 |
| Schooner | 0.8 | 0.8 | 7.7 | 0.5 | 19.0 | 28.2 | 37.4 |
| | | | | | 14.3 | 22.0 | 26.4 |
| Himalaya | 0.1 | 0.1 | 1.3 | 0.1 | 1.3 | 2.8 | 2.6 |
| | | | | | 1.2 | 2.5 | 2.3 |
| Waxiro | 0.7 | 0.8 | 7.1 | 0.6 | 15.8 | 21.8 | 28.3 |
| | | | | | 11.4 | 20.4 | 22.5 |
| Sloop | 0.6 | 0.6 | 7.0 | 0.4 | 10.1 | 17.4 | 18.9 |
| | | | | | 10.0 | 17.3 | 18.8 |
| Barley mutant 342 | 1.4 | 1.7 | 10.6 | 0.9 | 33.6 | 66.3 | 85.3 |
| | | | | | 42.7 | 93.8 | 121.9 |
| | | | | | 36.3 | 77.3 | 99.0 |
| Namoi | 0.6 | 0.6 | 7.0 | 0.4 | 8.7 | 16.4 | 16.5 |
| Tantangara | 0.5 | 0.5 | 7.6 | 0.3 | 10.6 | 20.1 | 21.8 |
| Tantangara × 292 | 1.9 | 3.2 | 11.0 | 1.0 | 41.1 | 95.4 | 119.5 |
| DH | | | | | | | |
| HA Glacier | 0.5 | 0.6 | 6.1 | 0.2 | 6.9 | 13.8 | 13.3 |
| Glacier | 0.8 | 0.7 | 5.7 | 0.5 | 7.3 | 14.3 | 14.0 |

^{a}Fructan content expressed as mg/g wholemeal, which is essentially mg/g grain

The effect on fructan levels of the SSIIa mutation in different genetic backgrounds was then examined. The SSIIa mutation was transferred by backcrossing (one cross and three backcrosses, with single seed descent, equivalent to BC3F4) to two different barley varieties, namely cultivars Sloop and Tantangara. Progeny lines 250 to 374 contained the SSIIa mutation in a barley Sloop background and lines 703 to 886 had a barley Tantangara background. K4 was a black barley grain with wildtype starch, without the SSIIa mutation, grown in the same field. Total sugars were extracted from 100 mg dry weight wholemeal as described above in section 4.1.

The results are shown in Table 4, which indicated that the SSIIa mutation in both the Tantangara and Sloop barley varieties produced a high fructan content (shown in mg/g wholemeal).

TABLE 4
Comparison of fructan contents in ten selected breeding lines

| | Un-hydrolysed | | | | | | |
|---|---|---|---|---|---|---|---|
| | Glucose | Fructose | Sucrose | | Hydrolysed | | |
| Barley | content | content | content | Maltose | Glucose | Fructose | Fructan |
| lines | (mg hexose equiv/g dry weight) | | | content | content | content | content |
| 250 | 2.3 | 2.4 | 13.4 | 1.7 | 20.2 | 48.5 | 48.9 |
| 348 | 2.6 | 2.9 | 14.9 | 1.4 | 16.7 | 43.7 | 38.6 |
| 363 | 2.3 | 2.8 | 13.3 | 1.2 | 19.9 | 49.1 | 49.4 |
| 374 | 2.5 | 3.3 | 13.9 | 1.6 | 20.0 | 52.5 | 51.2 |
| 703 | 2.2 | 2.7 | 12.9 | 1.1 | 20.5 | 54.1 | 55.6 |
| 871 | 1.8 | 2.1 | 13.5 | 1.5 | 13.2 | 41.3 | 35.6 |
| 926 | 2.3 | 3.2 | 12.7 | 1.2 | 20.3 | 52.6 | 53.4 |
| 930 | 2.1 | 2.9 | 11.9 | 1.1 | 13.9 | 48.8 | 44.6 |
| 886 | 1.5 | 2.3 | 12.9 | 1.6 | 18.6 | 46.4 | 46.6 |
| K4 | 0.4 | 0.4 | 6.7 | 0.2 | 4.2 | 8.8 | 5.2 |

### Analysis of Fructan Content in SSIIa Mutant Wheat Grain

Total sugars were extracted from 100 mg dry weight wholemeal as described above in section 4.1 for a wheat SSIIa triple null mutant (mutant in the A, B and D genomes of wheat) and the corresponding wildtype wheat Sunco. These were analysed for fructan content as for the barley grain.

The results (Table 5) indicated that the wheat grain of the SSIIa triple null mutant contained increased fructan levels compared to the corresponding wild-type wheat (Sunco) grain for all growing environments, but to a varying extent. For example, comparison between grain of the SSIIa null line B63 and the wild-type line B70 indicated a 2-3 fold increase in fructan in the mutant grain. The data in Table 5 also indicated that amounts of glucose, fructose, sucrose and maltose were sometimes increased in the null wheat. The data also showed that the wheat SSIIa triple null line had less hexoses than the control line BW26, but sucrose was not significantly altered. Overall, these results indicated that an SSIIa mutation increased fructan content not only in barley, but also in wheat.

TABLE 5
Analysis of fructan contents in wheat SSIIa mutant grain

| | Un-hydrolysed | | | | | | |
|---|---|---|---|---|---|---|---|
| | Glucose | Fructose | Sucrose | | Hydrolysed: | | |
| | content | content | content | Maltose | Glucose | Fructose | Fructan |
| Barley lines | (mg hexose equiv/g dry weight) | | | content | content | content | content |
| B29 GES 2003 | 1.17 | 1.06 | 10.08 | 1.96 | 11.35 | 25.32 | 22.39 |
| B63 GES 2003 | 0.58 | 0.52 | 13.33 | 1.64 | 17.99 | 40.14 | 42.06 |
| B70 GES 2003 wt^{a} | 0.21 | 0.14 | 4.34 | 0.14 | 5.73 | 12.49 | 13.39 |
| B23/24 GES 2004 (100) | 0.33 | 0.30 | 7.84 | 0.52 | 11.36 | 24.21 | 26.58 |
| B29 GES 2004 | 0.50 | 0.38 | 10.19 | 1.48 | 9.88 | 27.63 | 24.97 |
| B63 GES 2004 | 0.68 | 0.73 | 13.10 | 1.60 | 19.78 | 45.88 | 49.55 |
| A9 GES 2004 wt | 0.54 | 0.53 | 11.78 | 0.45 | 8.48 | 19.05 | 14.24 |
| A113 GES 2004 wt | 0.75 | 0.73 | 12.02 | 1.34 | 8.54 | 19.34 | 13.03 |
| B70 GES 2004 wt | 0.14 | 0.06 | 7.86 | 0.30 | 9.49 | 22.44 | 23.57 |
| B29 Griffith 2005 | 1.53 | 1.31 | 11.36 | 1.78 | 18.22 | 38.78 | 41.01 |
| B70 Griffith 2005 wt | 1.75 | 2.93 | 2.10 | 0.29 | 6.82 | 16.56 | 16.30 |
| B29 GES 2006 | 2.51 | 2.36 | 13.42 | 2.06 | 19.40 | 37.32 | 36.38 |
| B63 GES 2006 | 1.21 | 0.98 | 16.40 | 1.09 | 23.21 | 57.98 | 61.51 |
| A113 GES 2006 wt | 0.52 | 0.45 | 7.42 | 1.00 | 8.52 | 19.52 | 18.64 |
| B70 GES 2006 wt | 0.20 | 0.11 | 4.53 | 0.00 | 10.86 | 22.66 | 28.69 |

^{a}wt indicates these lines were wild-type for SSIIa

The lines used in this analysis were generated by crossing a parental SSIIa null wheat plants (SGP-1 null wheat) and wheat plants of cultivar Sunco. B29 GES 2003 and B63 GES 2003 were SSIIa mutant in all three genomes (triple nulls) and were grown at Ginninderra Experimental Station in 2003. B70 GES 2003 wt was wildtype for SSIIa and was also grown at Ginninderra Experimental Station in 2003. B23/24 GES 2004 (100), B29 GES 2004 and B63 GES 2004 were mutant for SSIIa (triple null) and were grown at Ginninderra Experimental Station in 2004. A9 GES 2004 wt, A113 GES 2004 wt and B70 GES 2004 wt were wildtype for SSIIa and were grown at Ginninderra Experimental Station in 2004. B29 Griffith 2005 was mutant for SSIIa (triple null) and was grown at Griffith in 2005. B70 Griffith 2005 wt was wildtype for SSIIa and was grown at Griffith in 2005. B29 GES 2006 and B63 GES 2006 were SSIIa mutant (triple nulls) and were grown at Ginninderra Experimental Station in 2006. A113 GES 2006 wt and B70 GES 2006 wt were wildtype for SSIIa and were grown at Ginninderra Experimental Station in 2006.

### Analysis of Fructan Content in High Amylose Barley Grain

Total sugars were extracted from 100 mg dry weight wholemeal of several barley lines transgenic for SBEIIa and/or SBEIIb RNAi constructs, and wild-type barley grain of cultivar Golden Promise. Those containing the SBEIIa RNAi constructs had strongly downregulated expression of the SBEIIa gene and consequently the grain starch in these lines was elevated to about 80% amylose.

The results indicated that the transgenic barley grain containing both the SBEIIa RNAi and SBEIIb RNAi constructs (transgenic barley #22) had elevated fructan levels compared to the wildtype barley grain (Golden Promise), but the transgenic lines containing either of the single constructs, transgenic barley #20, containing the SBEIIa RNAi construct and #21, containing the SBEIIb construct, were not elevated for fructan levels in the grain.

### Analysis of Fructan Content in High Amylose Wheat Grain

Total sugars were extracted from 100 mg dry weight wholemeal as described above in section 4.1 for a high amylose wheat line which was transgenic for an inhibitory SBEIIa RNAi construct and therefore elevated in amylose content to about 80% amylose (Regina et al., 2006 (supra)) and a corresponding wildtype wheat BW26. These were analysed for fructan levels in the grain as for the barley, above. SBEIIa and SBEIIb refer to starch branching enzymes IIa and IIb, respectively.

The results indicated that high amylose wheat grain containing the SBEIIa construct (#25, #26), a transgenic wheat line containing the SBEIIb construct (#27, not elevated in amylose level) and grain of a transgenic wheat line containing an SSI RNAi construct and having reduced starch synthase I activity did not have substantially increased fructan content compared to the wild-type wheat transgenic wheat line (#28).

### Effects of Growing Conditions on Fructan Content

Further experiments were carried out to determine the effects of growing conditions on the level of fructan in grain across a range of barley lines containing the SSIIa mutation.

Lines 250 to 374 and lines 703 to 926 were as described above. 2001-292: Barley M292 was grown at Ginninderra Experiment Station (GES), ACT in 2001.2001-292: Barley M292 was grown at Forbes (NSW) in 2001. 2002-292: Barley M292 was grown at Colleambally in 2002. 2003-292: Barley M292 was grown at Forbes in 2003. 2003-292: Barley M292 was grown at Colleambally in 2003.

The data showed that the fructan levels were elevated across a range of barley genetic backgrounds each containing the SSIIa mutation, and a variety of growing environments.

TABLE 6
Effects of growing conditions on fructan content

| | | Line | Fructan level |
|---|---|---|---|
| Line No | Growing site | name | (mg/g grain) |
| 2003 | Black mountain glasshouse, ACT | 250 | 107.1 |
| 2003 | Black mountain glasshouse, ACT | 266 | 53.0 |
| 2003 | Black mountain glasshouse, ACT | 348 | 59.8 |
| 2003 | Black mountain glasshouse, ACT | 363 | 115.2 |
| 2003 | Black mountain glasshouse, ACT | 374 | 98.3 |
| 2003 | Black mountain glasshouse, ACT | 703 | 174.2 |
| 2003 | Black mountain glasshouse, ACT | 871 | 99.6 |
| 2003 | Black mountain glasshouse, ACT | 886 | 126.9 |
| 2003 | Black mountain glasshouse, ACT | 926 | 103.9 |
| 2003 | Black mountain glasshouse, ACT | 930 | 164.0 |
| 2001 | Ginninderra Experimental Station, ACT | 292 | 68.9 |
| 2001 | Forbes | 292 | 76.6 |
| 2002 | Colleambally | 292 | 37.0 |
| 2003 | Forbes | 292 | 40.4 |
| 2003 | Colleambally | 292 | 42.1 |

### Example 5

### Large Scale Production of Fructan

Having about 10% fructan, the barley grain mutant in SSIIa can be used for the isolation and purification of fructan as well as other products such as high amylose starch and β-glucan. Such production from grain which can be readily produced in broadacre agriculture will be cost-effective relative to existing methods of fructan production, for example, involving the extraction of inulins from chicory. BarleyMax grain contained at least 5% fructan by weight, or when grown under some conditions, at least 10% fructan.

Large scale extraction of fructan can be achieved by milling the grain to wholemeal flour and then extracting the total sugars including fructans from the flour into water. This may be done at ambient temperature and the mixture then centrifuged or filtered. The supernatant is then heated to about 80° C. and centrifuged to remove proteins, then dried down. Alternatively, the extraction of flour can be done using 80% ethanol, with subsequent phase separation using water/chloroform mixtures, and the aqueous phase containing sugars and fructan dried and redissolved in water. Sucrose in the extract prepared either way may be removed enzymatically by the addition of α-glucosidase, and then hexoses (monosaccharides) removed by gel filtration to produce fructan fractions of various sizes. This would produce a fructan enriched fraction of at least 80% fructan.

### Example 6

### Production of Food Products

The mature grain as harvested, processed grain, wholemeal or flour obtained from the grain can be used to produce food products for consumption by humans or other mammals by any method known to persons of ordinary skill in the art. For example, wholemeal bread may be made by substituting from 15-30% (w/w) or even more of the wholemeal used in bread recipes with wholemeal from any one of the SSIIa mutant grain types described herein.

### Example 7

### Compositions for Treatment

A composition of purified fructan in the form of a capsule for oral treatment may be prepared by filling a standard two-piece hard gelatin capsule with 500 mg of the agent or compound, in powdered form, 100 mg of lactose, 35 mg of talc and 10 mg of magnesium stearate.

### Example 8

### In Vitro Fermentation Studies and Rat Feeding Trial

In vitro experiments and subsequent feeding trials in rats examined the fermentative and physiological properties of barley grain mutant 292 extracts.

### In Vitro Fermentation Studies

An anaerobic static batch culture system was used to model human colonic fermentation. This simulation system is widely used internationally and yields reliable and reproducible results. Inoculum for the system was freshly voided faeces sourced from healthy adult subjects consuming their habitual diets. After collection, faecal samples were homogenised and suspended at 10% w/v in sterile anaerobic PBS.

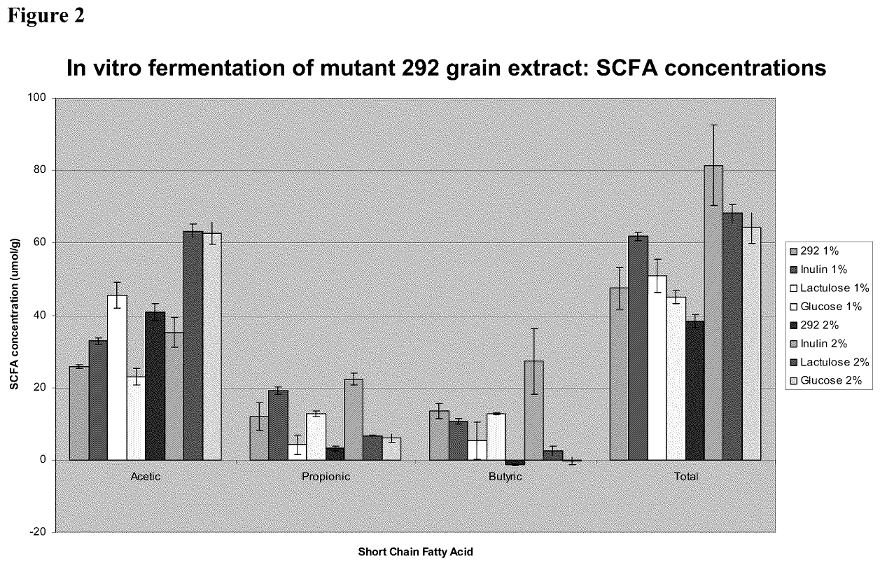

A series of studies employing a completely randomised experimental design was used to investigate the fermentative properties of novel cereal product (extracts of barley grain mutant 292). Predetermined amounts of these products were added to incubators. Reference carbohydrate substrates (glucose, lactulose and inulin), at comparable levels of addition, were included in each assay run. Quadruplicate incubations were performed in an anaerobic chamber for the cereal test products, standard substrates as well as for the Control (blank; no exogenous substrate added to the fermentor). Briefly, test products (and standards) were pre-weighed into sterile screw-capped sterile fermentation vessels and the carbon-limited fermentation media comprising carbonate buffer and macro- and micronutrients added to a achieve the predetermined volume and pH (7.0). After a period of equilibration an aliquot of inoculum was added to each fermentor. These were then capped, sealed and incubated at 37° C. with constant shaking. Incubations containing no added substrate (blank) were included in each assay run. After 24 hours, the ferments were frozen at −20° C. to await biochemical analysis using standardised procedures. Short chain fatty production (SCFA) of the test products and standards from the study are shown in FIG. 2. The results show that at the lower dosage (1%) the extracts of the barley grain mutant 292 produced a comparable fermentation pattern to that of inulin.

### Rat Feeding Trial

The rat study was designed to corroborate and extend the findings of the in vitro fermentation experiments. Such techniques are capable of providing only preliminary information as they are clearly unable to fully simulate the complex physical, microbial and chemical processes that occur along the gastrointestinal tract.

The primary aim of the study was to investigate the fermentation properties of extracts of barley grain mutant 292 stem. These novel products were compared against a negative control and an established (commercial) prebiotic.

The study comprised a completely randomised design involving 5 dietary treatment groups with 10 rats per group. The treatments were:

Negative control (basal diet containing no additional fibre); Barley grain mutant 292 extract, included at 2% and 5% (by weight) of the diet.

Positive controls: commercial oligofructose at 2% and 5% of the diet.

The major experimental endpoints were cecal SCFA concentrations and pools.

Briefly, rats (approximately 200 g live weight) were acclimatised for 7 days before being assigned randomly to treatment groups. A non-purified commercial chow was fed during the adaptation phase and treatment diets were fed for the subsequent 2 weeks of study. Rats were maintained in wire-based cages except for the final 4 days of study when they were kept in metabolic cages to enable record daily feed and water intakes, and fecal and urine output, of individual animals. Rats had unrestricted access to diets and drinking water throughout the study.

At the end of the treatment period rats were anaesthetized and various samples, including intestinal contents, collected and stored to await biochemical analysis using standardized laboratory techniques.

The diets that were fed were based on the AlN-93G formulation and as such comprised a uniform composition of macronutrients and fibre as NSP (at ˜15% w/w). A vitamin and mineral mixture added to ensure that all diets were nutritionally complete. Four of the 5 diets contained oligosaccharide extracts from barley or chicory added at either 2% or 5% (by weight of diet).

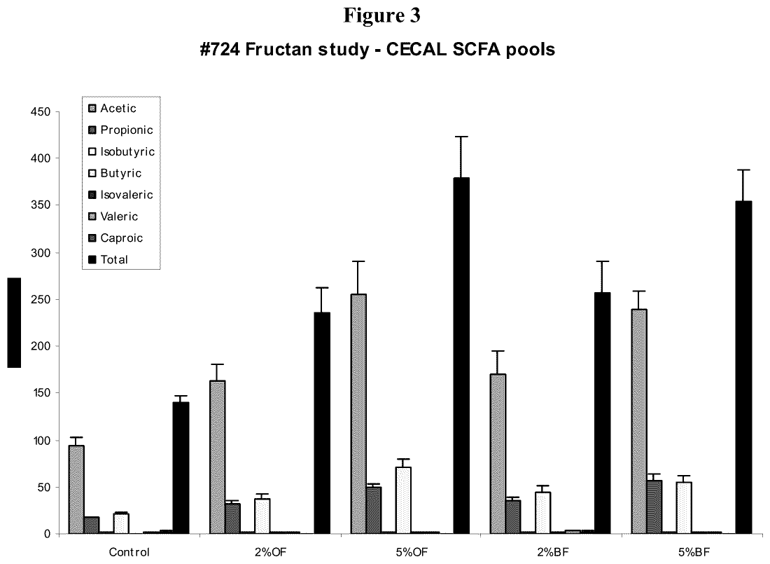

Results: Food intake was similar among treatment groups, although final live weights were greater for all treatment groups compared to the control. Fecal output was greater for all treatment groups relative to controls and related positively to level of dietary inclusion of the various extracts. Treatment differences in caecal digesta weight mirrored those for faecal output. Consumption of treatment extracts was associated with acidification of caecal digesta. FIG. 3 shows caecal pools of individual and total SCFA. The total amount of SCFA was greater for the treatments compared to controls, and the responses to barley grain extract (at the corresponding level of dietary inclusion) were comparable to those of oligofructose. In summary, the barley grain mutant 292 extracts produced comparable results to those of the corresponding oligofructose standards.

Those skilled in the art will appreciate that the invention described herein is susceptible to variations and modifications other than those specifically described. It is to be understood that the invention includes all such variations and modifications. The invention also includes all of the steps, features, compositions and compounds referred to or indicated in this specification, individually or collectively, and any and all combinations of any two or more of said steps or features.

TABLE 7
Summary of sequence identifiers

| SEQUENCE | |
|---|---|
| ID NO: | DESCRIPTION |
| 1 | Hordeum vulgare subsp. vulgare starch synthase II |
| | mRNA, complete cDNA sequence. Accession No. |
| | AY133249, 2972 nucleotides, protein coding region: |
| | nucleotides 114-2522, on chromosome 7 of barley. |
| 2 | amino acid sequence of starch synthase II encoded by |
| | SEQ ID NO: 1; 802 amino acids |
| 3 | Triticum aestivum starch synthase IIa mRNA, complete |
| | cDNA sequence, Accession No. AF155217. 2842 |
| | nucleotides, protein coding region: nucleotides 89-2488 |
| | Reference: Li et al., Plant Physiol. 120: 1147-1156, |
| | 1999. |
| 4 | amino acid sequence of starch synthase IIa encoded by |
| | SEQ ID NO: 3: 799 amino acids |
| 5 | Triticum aestivum cDNA sequence for starch synthase |
| | IIa-2 (wSSIIa-2 gene). Accession No. AJ269503. 2780 |
| | nucleotides, protein coding region: nucleotides 55-2454; |
| | mature peptide: nucleotides 230-2451 |
| | Reference: Gao and Chibbar, Genome 43: 768-775, |
| | 2000. |
| 6 | Triticum aestivum amino acid sequence encoded by |
| | wSSIIa gene for starch synthase IIa (EC. 2.4.1.21), |
| | precursor 799 amino acids. |
| 7 | Triticum aestivum wSSIIa-B gene for starch synthase |
| | IIa, genomic sequence from B genome, Accession No. |
| | AB201446, exons: 246-506, 611-1316, 1407-1471, |
| | 2287-2364, 2454-2564, 2662-2706, 3085-3258, 5855-6811. |
| | Reference: Shimbata et al., Theor. Appl. Genet. 111: |
| | 1072-1079, 2005. |
| 8 | amino acid sequence of SSIIa-B encoded by SEQ ID |
| | NO: 7: 798 amino acids |
| 9 | Triticum aestivum wSSIIa-D gene for starch synthase |
| | IIa-D, predicted amino acid sequence, Accession No. |
| | AB201447: 799 amino acids |
| 10 | Sorghum bicolor starch synthase IIa complete cDNA |
| | sequence, Accession No. EU620718, 2400 nucleotides, |
| | protein coding region: 31-2262 |
| 11 | amino acid sequence encoded by nucleotides 31 to 2262 |
| | of SEQ ID NO: 10 |
| 12 | Oryza sativa mRNA for starch synthase IIa, complete |
| | cDNA sequence, Accession No. AB115918, 2433 |
| | nucleotides, protein coding region: 1-2433. |
| | Reference: Nakamura et al., Plant Mol. Biol. 58: 213-227, 2005. |
| 13 | amino acid sequence encoded by nucleotides 1 to 2432 |
| | of SEQ ID NO: 12: 810 amino acids |
| 14 | Zea mays ZmSSIIa mRNA sequence, Accession No: |
| | BT023979, 2248 nucleotides |
| | Reference: Lai et al., Genome Res. 14: 1932-1937, |
| | 2004. |
| 15 | Oryza sativa SSIIb sequence of cDNA clone: |
| | Accession No. AK066446, 2645 nucleotides, |
| | Reference: Yamakawa et al., Plant Physiol 144: 258-277, 2007. |
| 16 | Oryza sativa soluble starch synthase II-2 mRNA, |
| | complete cDNA sequence, Accession No. AF395537, |
| | 2394 nucleotides, protein coding region: nucleotides 34-2118. |
| 17 | amino acid sequence encoded by nucleotide of 34-2118 |
| | of SEQ ID NO: 16. Accession No. AAK81729, 694 |
| | amino acids |
| 18 | Triticum aestivum starch synthase IIb precursor, cDNA |
| | sequence, 2025 nucleotides Accession No. EU333947, |
| | protein coding region: 1-2025. |
| 19 | amino acid sequence encoded by nucleotide 1 to 2025 of |
| | SEQ ID NO: 18: 674 amino acids |
| 20 | Sorghum bicolor starch synthase IIb precursor, mRNA, |
| | complete cDNA sequence, Accession No. EU620719, |
| | 2302 nucleotides, protein coding region: 36-2150. |
| 21 | amino acid sequence encoded by nucleotide 1 to 2025 of |
| | SEQ ID NO: 20: 704 amino acids |
| 22 | Zea mays starch synthase IIb- precursor, mRNA, |
| | complete cDNA sequence, Accession No. EF472249, |
| | protein coding region nucleotides 74-2188, 2569 |
| | nucleotides |
| 23 | amino acid sequence encoded by nucleotide 1 to 2025 of |
| | SEQ ID NO: 22: 704 amino acids |

TABLE 8
Amino acid sub-classification

| Sub-classes | Amino acids |
|---|---|
| Acidic | Aspartic acid, Glutamic acid |
| Basic | Noncyclic: Arginine, Lysine; Cyclic: Histidine |
| Charged | Aspartic acid, Glutamic acid, Arginine, Lysine, Histidine |
| Small | Glycine, Serine, Alanine, Threonine, Proline |
| Polar/neutral | Asparagine, Histidine, Glutamine, Cysteine, Serine, |
| | Threonine |
| Polar/large | Asparagine, Glutamine |
| Hydrophobic | Tyrosine, Valine, Isoleucine, Leucine, Methionine, |
| | Phenylalanine, Tryptophan |
| Aromatic | Tryptophan, Tyrosine, Phenylalanine |
| Residues that influence | Glycine and Proline |
| chain orientation | |

TABLE 9
Exemplary and Preferred Amino Acid Substitutions

| Original | EXEMPLARY | PREFERRED |
|---|---|---|
| Residue | SUBSTITUTIONS | SUBSTITUTIONS |
| Ala | Val, Leu, Ile | Val |
| Arg | Lys, Gln, Asn | Lys |
| Asn | Gln, His, Lys, Arg | Gln |
| Asp | Glu | Glu |
| Cys | Ser | Ser |
| Gln | Asn, His, Lys, | Asn |
| Glu | Asp, Lys | Asp |
| Gly | Pro | Pro |
| His | Asn, Gln, Lys, Arg | Arg |
| Ile | Leu, Val, Met, Ala, Phe, | Leu |
| | Norleu | |
| Leu | Norleu, Ile, Val, Met, Ala, Phe | Ile |
| Lys | Arg, Gln, Asn | Arg |
| Met | Leu, Ile, Phe | Leu |
| Phe | Leu, Val, Ile, Ala | Leu |
| Pro | Gly | Gly |
| Ser | Thr | Thr |
| Thr | Ser | Ser |
| Trp | Tyr | Tyr |
| Tyr | Trp, Phe, Thr, Ser | Phe |
| Val | Ile, Leu, Met, Phe, Ala, Norleu | Leu |

### BIBLIOGRAPHY

Adams et al., Anal. Biochem., 266: 77-84, 1999
Almeida and Allshire, Trends Cell Biol. 15: 251-258, 2005
Altschul et al., Nucleic Acids Res. 25: 3389, 1997
An, Methods in Enzymology, 153: 292, 1987
Appels and Dvorak, Theoretical and Applied Genetics, 63: 337-348, 1982
Ausubel et al., (eds.), Current Protocols in Molecular Biology, John Wiley & Sons, NY, 6.3.1-6.3.6., 1989
Ausubel et al., Current Protocols in Molecular Biology, John Wiley & Sons Inc, Chapter 15, 1994-1998
Barker et al., Plant Mol. Biol., 2: 235-350, 1983
Batey et al., J. Sci. Food Agric. 74: 503-508, 1997
Bechtold et al., C.R. Acad. Sci. Paris, 316: 1194, 1993
Bernfeld, In: Colowick S, Kaplan N (eds), Methods in enzymology. Academic, NY, p 149, 1955
Bevan et al., Nucl. Acid Res., 11: 369, 1983
Birch, Ann Rev Plant Physiol Plant Mol Biol. 48: 297-326, 1997
Bird et al. Br. J. Nutr. 92: 607-615, 2004b
Bird et al., J. Nutr. 134: 831-835, 2004a
Bourque, Plant Sci. 105: 125-149, 1995
Boyer and Preiss, Carbohydrate Research, 61: 321-334, 1978
Buléon et al., International Journal of Biological Macromolecules, 23: 85-112, 1998
Campbell et al., Journal of the science offood and agriculture 79: 232-236, 1999
Cao et al., Archives of Biochemistry and Biophysics, 373: 135-146, 2000
Cao et al., Plant Physiol. 120: 205-215, 1999
Christensen et al., Transgen. Res., 5: 213-218, 1996
Clarke and Rahman, Theoretical and Applied Genetics, 110: 1259-1267, 2005
Comai et al., Plant J. 37: 778-786, 2004
Craig et al., Plant Cell 10: 413-426, 1998
De Framond, Biotechnology, 1: 262, 1983
Deikman et al., EMBO J., 2: 3315-3320, 1998
DellaPenna et al., Plant Cell, 1: 53-63, 1989
Eagles et al., Aust. J. Agric. Res. 52: 1 349-1356, 2001
Fincher and Stone, Advances in Cereal Science and Technol 8: 207-295,1986
Fromm et al., Proc. Natl. Acad. Sci. U.S.A. 82: 5824, 1985
Gao and Chibbar, Genome 43: 768-775, 2000
Gao et al., Plant Cell, 10: 399-412, 1998
Garfinkel et al., Cell, 27: 143-153, 1983
Greve, J. Mol. Appl. Genet., 1: 499-511, 1983
Guerin et al., Journal of Cereal Science, 15: 5-14, 1992
Harayama, Trends Biotechnol. 16: 76-82, 1998
Hartmann and Endres, Manual of Antisense Methodology, Kluwer, 1999
Harvey et al., Physiologia Plantarum 113: 108-120, 2001
Haseloff and Gerlach, Nature 334: 585-591, 1988
Hedman and Boyer, Biochemical Genetics, 20: 483-492, 1982
Hellwege et al., Proc. Natl. Acad. Sci. U.S.A. 97: 8699-8704, 2000
Hendrix et al., J. Insect Physiol., 47: 423-432, 2001
Henikoff et al., Plant Physiol. 135: 630-636, 2004
Henry and Saini, Cereal Chem. 66: 362-365, 1989
Hinchee et al., Biotech. 6: 915, 1988
Hoekema et al., Nature, 303: 179, 1983
Hrmova and Fincher, Plant Molecular Biology, 47: 73-91, 2001
James et al., Plant Cell, 7: 417-429, 1995
Joshi, Nucl. Acid Res. 15: 6643, 1987
Kaur and Gupta, J. Biosci. 27: 703-714, 2002
Khandjian, Bio/Technology, 5: 165-167, 1987
Klein et al., Nature, 327: 70, 1987
Konik-Rose et al. Starch/die Stdrke 53:14-20, 2001
Kubo et al., Plant Physiology, 121: 399-409, 1999
Lai et al., Genome Res. 14: 1932-1937, 2004
Langridge et al., Aust. J. Agric. Res. 52: 1043-1077, 2001
Lemieux, Current Genomics, 1: 301-311, 2000.
Li et al., Plant Physiology, 120: 1147-1155, 1999a
Li et al., Plant Physiology, 123: 613-624, 2000
Li et al., Theoretical and Applied Genetics, 98: 1208-1216, 1999b
Libessart et al. Plant Cell 7(8): 1117-1127, 1995
Lunn and Hatch, Planta 197: 385-391, 1995
MacGregor et al., Cereal Chemistry, 48: 255-269, 1971
McPherson and Moller (Ed), BIOS Scientific Publishers Ltd, Oxford, 2000
Medberry et al., Plant Cell, 4: 185-192, 1992
Medberry et al., Plant J. 3: 619-626, 1993
Millar and Waterhouse, Funct Integr Genomics, 5: 129-135, 2005
Mizuno et al., Journal of Biochemistry, 112: 643-651, 1992
Morell et al., Electrophoresis, 19: 2603-2611, 1998
Morell et al., Plant J. 34: 173-185, 2003
Moshfegh et al., JNutr. 129(Suppl): 1407S-11S, 1999
Myers et al., Plant Physiology, 122: 989-997, 2000
Nakamura et al., Plant Mol. Biol. 58: 213-227, 2005
Needleman and Wunsch, J. Mol. Biol. 48: 443-453, 1970
Niedz et al., Plant Cell Reports, 14: 403, 1995
Ow et al., Science, 234: 856, 1986
Pasquinelli et al., Curr Opin Genet Develop 15: 200-205, 2005
Perriman et al., Gene, 113: 157-163, 1992
Potrykus et al., Mol. Gen. Genet. 199: 183, 1985
Prasher et al., Biochem. Biophys. Res. Comm. 126: 1259-68, 1985
Prosky et al., JAssoc OffAgric Chem 68: 677, 1985
RACI, Method 02-03, 2003
Regina et al., Proc. Natl. Acad. Sci. U.S.A. 103: 3546-3551, 2006
Remington's Pharmaceutical Sciences, 18^{th} Ed. Mack Publishing, Company, Easton, PA, U.S.A., 1990
Ritsema and Smeekens, Curr. Opin. Plant Biol. 6: 223-230, 2003
Robinson, The Organic Constituents of Higher Plants, Cordus Press, North Amherst, USA, 1980
Ruuska et al., Functional Plant Biology 33: 799-809, 2006
Salomon et al., EMBO J., 3: 141-146, 1984
Sambrook et al., Molecular Cloning: A Laboratory Manual (2nd ed.). Cold Spring Harbour Laboratory, Cold Spring Harbour, NY, 1989
Schenk et al., Proc. Natl. Acad. Sci. U.S.A. 97: 11655-11660, 2000
Schnyder, New Phytol 123: 233-245, 1993
Schulman and Kammiovirta, Starch, 43: 387-389, 1991
Senior, Biotech. Genet. Engin. Revs. 15: 79-119, 1998
Sevenier et al., Nature Biotechnol. 16: 843-846, 1998
Sharp et al., Aust JAgric Res 52: 1357-1366, 2001
Shimamoto et al., Nature, 338: 274-276, 1989
Shimbata et al., Theor. Appl. Genet. 111: 1072-1079, 2005
Shippy et al., Mol. Biotech. 12: 117-129, 1999
Slade and Knauf, Transgenic Res. 14: 109-115, 2005
Smith et al., Nature, 407: 319-320, 2000
Stalker et al., Science, 242: 419, 1988
Sun et al., The New Phytologist, 137: 215-215, 1997
Theander et al., JAOAC Int 78: 1030-1044, 1995
Thillet et al., J. Biol. Chem. 263: 12500, 1988
Thompson et al., Carbohydrate Res., 331: 149-161, 2001
Tingay et al., Plant J. 11: 1369-1376, 1997
Topping et al., Starch/Stdrke 55: 539-545, 2003
Tsuchiya et al., Physiologia Plantarum 125: 181-191, 2005
Tungland and Meyer, Comprehensive Reviews in Food Science and Food Safety, 2: 73-77, 2002
Van den Ende et al., Plant Physiol. 131(2): 621-631, 2003
Veronese et al., Enz. Microbial Tech., 24: 263-269, 1999
Wan and Lemaux, Plant Physiol. 104: 37-48, 1994
Waterhouse et al., Proc. Natl. Acad. Sci. U.S.A. 95: 13959-13964, 1998
White and Secor, Arch Biochem Biophys. 44: 244-5, 1953
Wilson et al., Bioinformatics 19: 1325-1332, 2003
Winter and Huber, Critical Reviews in Plant Sciences 19: 31-67, 2000
Yamakawa et al., Plant Physiol 144: 258-277, 2007
Yamamori et al., Theor. Appl. Genet. 101: 21-29, 2000
Zwar and Chandler, Planta 197: 39-48, 1995.

The claims defining the invention are as follows:

1. A method of producing a high-fructan product, wherein the method comprises:
(i) obtaining or producing a cereal plant or grain or flour therefrom wherein the cereal grain or flour comprises at least 3%, preferably at least 4%, fructan as a percentage of the cereal grain or flour weight;
(ii) processing the plant, grain or flour to produce the product.

2. A method of producing a high-fructan product, wherein the method comprises:
(i) obtaining or producing a cereal plant or grain or flour therefrom wherein the cereal plant or grain is modified to comprise a reduced level of an endogenous polypeptide with starch synthase II (SSII) activity relative to an unmodified control;
(ii) processing the plant, grain or flour to produce the product.

3. The method of claim 1 or 2 further comprising:
(i) assessing the level or type of fructan in the cereal plant or grain or flour therefrom, or the product therefrom.

4. The method of claim 1, 2 or 3 wherein the cereal grain is wholegrain.

5. The method of any one of claims 1 to 4 wherein the cereal grain is cracked, ground, polished, milled, kibbled, rolled or pearled grain.

6. The method of any one of claims 1 to 5 wherein the starch content of the grain is at least 30%, preferably at least 35%, or at least 40% as a percentage of the total grain weight.

7. The method of any one of claims 1 to 6 wherein the cereal plant is selected from the group consisting of barley, wheat, rice, maize, rye, oat and sorghum.

8. The method of claim 7 wherein the plant is tetraploid wheat, maize, rye, rice, oat or sorghum.

9. The method of claim 7 wherein the plant is hexaploid wheat or barley.

10. The method of claim 9 wherein the barley is a barley shrunken grain mutant designated M292 or M342.

11. The method of any one of claims 1 to 10 wherein the fructan comprises a degree of polymerization from about 3 to about 12.

12. The method of any one of claims 1 to 11 wherein the product is a food or beverage product or a pharmaceutical composition.

13. The method of any one of claims 1 to 12 wherein the product is isolated fructan.

14. The method of claim 12 wherein the food or beverage product is a grain, flour, breakfast cereal, biscuit, muffin, muesli bar, noodle, corn, a sweetening agent, a low calorie additive, a bulking agent, a dietary fibre, a texturizing agent, a preservative, a probiotic agent or the like or any combination of these.

15. The method of any one of claims 1 to 14 wherein the cereal plant or grain is modified to comprise a reduced level of an endogenous polypeptide with starch synthase II (SSII) activity relative to an unmodified control.

16. The method of claim 15 wherein the plant comprises a mutation in an endogenous gene encoding a polypeptide with SSII activity wherein the mutation reduces the expression of the gene encoding SSII in the plant or leads to the expression of SSII with reduced level or activity.

17. The method of claim 15 wherein the level of SSII activity is reduced by introducing into said plant a nucleic acid molecule which down-regulates the expression of a gene encoding SSII in the plant.

18. The method of claim 17 wherein the nucleic acid molecule comprises a gene-silencing chimeric gene, an antisense, ribozyme, co-suppression, dsRNA molecule, hairpin RNA molecule or other exogenous nucleic acid molecule that down-regulates endogenous SSII expression.

19. A method of producing a cereal plant or grain therefrom which has increased levels of fructan compared to a control plant, wherein the method comprises:
(i) introducing into said plant an agent which down-regulates the level or activity of endogenous starch synthase II (SSII) in the plant relative to a control plant, or a mutation in an endogenous gene encoding SSII in the plant.

20. The method of claim 19 wherein the agent comprises a nucleic acid molecule which down-regulates endogenous SSII gene expression.

21. The method of claim 20 wherein the nucleic acid molecule comprises a gene-silencing chimeric gene, an antisense, ribozyme, co-suppression, dsRNA molecule, hairpin RNA or other exogenous nucleic acid molecule that down-regulates endogenous SSII expression.

22. The method according to any one of claims 19 to 21 wherein the method further comprises assessing the level, activity or type of fructan in the plant or grain therefrom.

23. The method of any one of claims 19 to 22 wherein the cereal grain is wholegrain.

24. The method of any one of claims 19 to 23 wherein the cereal grain is cracked, ground, polished, milled, kibbled, rolled or pearled grain.

25. The method of any one of claims 19 to 24 wherein the starch content of the grain is at least 30%, preferably at least 35%, or at least 40% as a percentage of the total grain weight.

26. The method of any one of claims 19 to 25 wherein the plant is selected from the group consisting of barley, wheat, rice, maize, rye, oat and sorghum.

27. The method of claim 26 wherein the plant is tetraploid wheat, maize, rye, rice, oat or sorghum.

28. The method of claim 26 wherein the plant is hexaploid wheat or barley.

29. The method of claim 28 wherein the barley is a barley shrunken grain mutant designated M292 or M342.

30. The method of any one of claims 19 to 29 wherein the increased level of fructan is at least twice, preferably at least 3×, at least 4×, at least 5×, at least 6×, at least 7×, at least 8×, at least 9× or at least 10×, that of a control plant or the plant prior to the introduction of the agent or mutation.

31. An isolated or genetically modified cereal plant or grain therefrom or flour therefrom wherein the grain or flour comprises at least 3%, preferably at least 4% fructan as a percentage of the cereal grain or flour weight.

32. The plant or grain or flour of claim 31 when used or for use in the production of a product to increase the level of fructan or non-starch carbohydrate in said product.

33. The cereal grain of claim 31 or 32 wherein the grain is wholegrain.

34. The cereal grain of claim 31 or 32 which is cracked, ground, polished grain, milled, kibbled, rolled or pearled grain.

35. The plant of any one of claims 31 to 34 wherein the starch content of the grain is at least 30%, preferably at least 35%, or at least 40% as a percentage of the total grain weight.

36. The plant of claim 31, 32 or 35 wherein the cereal plant is selected from the group consisting of barley, wheat, rice, maize, rye, oat and sorghum.

37. The plant of claim 36 wherein the plant is tetraploid wheat, maize, rye, rice, oat, or sorghum.

38. The plant of claim 36 wherein the plant is hexaploid wheat or barley.

39. The plant of claim 38 wherein the barley is a barley shrunken grain mutant designated M292 or M342.

40. Cereal grain, flour or fructan produced from the plant or grain of any one of claims 31 to 39.

41. The cereal grain or flour of any one of claims 31 to 34 or 40 wherein the starch content is at least 30%, preferably at least 35%, or at least 40% as a percentage of the total grain weight.

42. The cereal grain of any one of claims 31 to 34, 40 or 41 wherein the cereal plant is not barley or hexaploid wheat.

43. The cereal grain or flour of any one of claims 31 to 34, or 40 to 42 comprising a reduced level or activity of a polypeptide having SSII activity.

44. Fructan, grain or flour produced from the plant or grain or flour of any one of claims 31 to 43.

45. Use of fructan isolated from a plant, grain or flour of any one of claims 31 to 44 in a food as a sweetening agent, a low calorie additive, a bulking agent, a dietary fibre, a texturizing agent, a preservative, a probiotic agent or the like or any combination of these.

46. Use of a grain or flour or frunctan isolated from a plant, grain or flour of any one of claims 31 to 44 in the production of a food product to increase the level of frunctan in said food product.

47. A food product comprising a food ingredient at a level of at least 10% on a dry weight basis, wherein the food ingredient is a cereal grain comprising at least 3%, preferably at least 4%, fructan on a weight basis or wholemeal or flour obtained therefrom wherein the wholemeal or flour comprises at least 3%, preferably at least 4% fructan on a weight basis.

48. A method of identifying a variety of cereal grain which has increased levels of fructan comprising:
(i) obtaining cereal grain which is altered in starch via synthesis or catabolism,
(ii) determining the amount of fructan in the grain,
(iii) comparing the level of fructan to that in wild-type grain which is not altered in starch via synthesis or catabolism, and
(iv) if the fructan level is increased in the altered grain, selecting the grain.

49. The method of claim 48 which further comprises mutagenesis or plant cell transformation prior to step (i).

50. A method of determining the amount of fructan in cereal grain, comprising the step of obtaining grain comprising at least 3%, preferably at least 4% fructan on a weight basis, processing the grain so as to extract the fructan, and measuring the amount of extracted fructan so as to determine the amount of fructan in the grain.

51. The product or method of any one of claims 47 to 50 wherein the cereal grain is wholegrain.

52. The product or method of any one of claims 47 to 50 wherein the cereal grain is cracked, ground, polished, milled, kibbled, rolled or pearled grain.

53. The product or method of any one of claims 47 to 50 wherein the starch content of the grain is at least 30%, preferably at least 35%, or at least 40% as a percentage of the total grain weight.

54. The product or method of claim 53 wherein the plant is selected from the group consisting of barley, wheat, rice, maize, rye, oat and sorghum.

55. The product or method of claim 54 wherein the plant is tetraploid wheat, maize, rye, rice, oat or sorghum.

56. The product or method of claim 54 wherein the plant is hexaploid wheat or barley.

57. The product or method of claim 56 wherein the barley is a barley shrunken grain mutant designated M292 or M342.

58. A method for preparing a food or beverage, comprising mixing a high-fructan product obtain by the method of any one of claims 1 to 18 with another food or beverage ingredient.

59. A method for providing fructan to improve one or more indicators of health in a mammal, wherein the method comprises administering, to the mammal, a composition comprising cereal grain or flour therefrom comprising at least 3%, preferably at least 4%, fructan on a weight basis or fructan obtained therefrom.

60. The method of claim 59 wherein the grain, flour or fructan is in the form of a food product, a beverage or a pharmaceutical composition.

61. The method of claim 59, wherein the one or more indicators of health is an increased number of beneficial intestinal bacteria, reduced number of aberrant crypt foci, increased mineral absorption, reduced level of insulin, reduced glycaemic index, reduced glycaemic load, reduced blood glucose, reduced blood pressure, reduced body weight, reduced blood cholesterol level, increased HDL cholesterol level, increased bone density, increased calcium levels, more frequent bowel movement, or improved blood serum cardiovascular profile.

62. A method for ameliorating one or more symptoms of a condition associated with low levels of dietary fructan in a subject, said method comprising administering orally to the subject grain comprising at least 3%, preferably at least 4%, fructan as a percentage of the cereal grain weight or a processed product comprising the fructan obtained therefrom for a time and under conditions sufficient to ameliorate one or more symptoms.

63. The method of claim 62 wherein the condition is selected from the group consisting of diabetes, obesity, heart disease, hypertension, constipation, osteoporesis and cancer.

64. The method of claim 62 wherein the subject is a human.
